SEQUENCE LISTING (5 sequences)

SEQ ID NO: 1 - DNA, 1731 bp, Homo sapiens
  CDS (271)..(1494)
  ref <301>: AUTHORS: Plengvidhya et al.
  ref <302>: TITLE: Hepatocyte nuclear factor 4 gamma: cDNA sequence, gene organization, and mutation screening in early-onset, autosomal dominant type 2 diabetes
  ref <303>: JOURNAL: Diabetes
  ref <304>: VOLUME: 48
  ref <305>: ISSUE: 10
  ref <306>: PAGES: 2099-2102
  ref <307>: DATE: 1999-10-01
  ref <308>: DATABASE ACCESSION NUMBER: GenBank/GI:6631087
  ref <309>: DATABASE ENTRY DATE: 2000-11-01
  ref <313>: RELEVANT RESIDUES: (1)..(1731)
  ref <308>: DATABASE ACCESSION NUMBER: GenBank/Z49826
  ref <309>: DATABASE ENTRY DATE: 1999-09-01
  ref <313>: RELEVANT RESIDUES: (1)..(500)
ggagcctcggttcccctcgggcggctgaaggaggggctgagcctcaggacccctaggccaggccgtgttgaaagcagataccccgaggcctcgcccagcctgcgctggagggaagaaggcgctggaggagcaccagcgaaagcagccagtctgagatattgacactacagaaaaaactgacagcttactccttgtattgatgctactcttctctacaaatatagactccgttccctaccacagccttatagttctgccccagagacaagtatgaataccacagacaacggtgtcaactgtctgtgtgctatctgtggggacagagcaacaggaaaacactatggggcatccacctgtgatgggtgcaagggtttcttcagacgcagcattcgtaagagtcacatttattcttgcaggttcagtcggcaatgtgttgttgacaaggacaaaaggaatcaatgtagatattgtcgattaagaaagtgttttagagcgggaatgaaaaaagaagctgtacaaaatgaacgtgacagaataagcaccagaagaagcacatttgatggcagcaacatcccctccattaacacactggcacaagctgaagttcggtctcgccagatctcagtctcaagccctgggtcaagcactgacataaacgttaagaaaattgcaagtattggtgatgtctgtgaatctatgaaacagcagctcttagtcttggtggaatgggctaaatatattcctgccttctgtgaattaccattggatgatcaggtggcactgttgagagctcacgcaggggagcacttactgcttggagctacaaagagatccatgatgtataaagatattttgcttttgggaaacaactatgttattcaccgcaacagctgtgaagttgagattagccgtgtggccaatcgtgttctagatgagctggttagaccatttcaagaaatccagattgatgacaatgagtatgcttgtttaaaggcaattgtattttttgatccagatgcaaaagggctaagcgatccagtaaaaattaagaacatgaggttccaagtgcagatcggtttggaggactacatcaatgatcggcagtatgactcccgggggaggtttggagagttgcttctgctcctgcccacactgcagagcatcacgtggcaaatgattgagcaaatacagtttgttaaactttttgggatggttaaaattgacaatctacttcaggaaatgctattaggtggggcttccaatgatggcagtcatctccatcatccaatgcatccacatttgtctcaagacccattaactggacaaactatacttttaggtcccatgtcaacactggttcatgcagaccagatctcaactcctgaaaccccactcccttccccaccacaaggctctgggcaagaacagtacaaaatagctgcaaaccaagcatcagtcatttcacaccagcatctctccaaacaaaagcaattgtgaaaatgtgtttacttcagaacggcactacataaatgtgaaaagttgttgatcttgaaatatctcaagatagcacttttggcaaactcttagccaaggcttcttcattggtgctgttataagatggtatcctattttcttgtttatacgttcattctatttgttattgctactatgtgaaactttcacatgcaaccaatgtatatctgagtttgaaggatgtttatatagggtagg

SEQ ID NO: 2 - protein, 408 aa, Homo sapiens
MNTTDNGVNCLCAICGDRATGKHYGASTCDGCKGFFRRSIRKSHIYSCRFSRQCVVDKDKRNQCRYCRLRKCFRAGMKKEAVQNERDRISTRRSTFDGSNIPSINTLAQAEVRSRQISVSSPGSSTDINVKKIASIGDVCESMKQQLLVLVEWAKYIPAFCELPLDDQVALLRAHAGEHLLLGATKRSMMYKDILLLGNNYVIHRNSCEVEISRVANRVLDELVRPFQEIQIDDNEYACLKAIVFFDPDAKGLSDPVKIKNMRFQVQIGLEDYINDRQYDSRGRFGELLLLLPTLQSITWQMIEQIQFVKLFGMVKIDNLLQEMLLGGASNDGSHLHHPMHPHLSQDPLTGQTILLGPMSTLVHADQISTPETPLPSPPQGSGQEQYKIAANQASVISHQHLSKQKQL

SEQ ID NO: 3 - DNA, 921 bp, Homo sapiens
  CDS (1)..(921)
  ref <308>: DATABASE ACCESSION NUMBER: GenBank/Z49826
  ref <309>: DATABASE ENTRY DATE: 1999-09-01
ccctccattaacacactggcacaagctgaagttcggtctcgccagatctcagtctcaagccctgggtcaagcactgacataaacgttaagaaaattgcaagtattggtgatgtctgtgaatctatgaaacagcagctcttagtcttggtggaatgggctaaatatattcctgccttctgtgaattaccattggatgatcaggtggcactgttgagagctcacgcaggggagcacttactgcttggagctacaaagagatccatgatgtataaagatattttgcttttgggaaacaactatgttattcaccgcaacagctgtgaagttgagattagccgtgtggccaatcgtgttctagatgagctggttagaccatttcaagaaatccagattgatgacaatgagtatgcttgtttaaaggcaattgtattttttgatccagatgcaaaagggctaagcgatccagtaaaaattaagaacatgaggttccaagtgcagatcggtttggaggactacatcaatgatcggcagtatgactcccgggggaggtttggagagttgcttctgctcctgcccacactgcagagcatcacgtggcaaatgattgagcaaatacagtttgttaaactttttgggatggttaaaattgacaatctacttcaggaaatgctattaggtggggcttccaatgatggcagtcatctccatcatccaatgcatccacatttgtctcaagacccattaactggacaaactatacttttaggtcccatgtcaacactggttcatgcagaccagatctcaactcctgaaaccccactcccttccccaccacaaggctctgggcaagaacagtacaaaatagctgcaaaccaagcatcagtcatttcacaccagcatctctccaaacaaaagcaattg

SEQ ID NO: 4 - protein, 307 aa, Homo sapiens
PSINTLAQAEVRSRQISVSSPGSSTDINVKKIASIGDVCESMKQQLLVLVEWAKYIPAFCELPLDDQVALLRAHAGEHLLLGATKRSMMYKDILLLGNNYVIHRNSCEVEISRVANRVLDELVRPFQEIQIDDNEYACLKAIVFFDPDAKGLSDPVKIKNMRFQVQIGLEDYINDRQYDSRGRFGELLLLLPTLQSITWQMIEQIQFVKLFGMVKIDNLLQEMLLGGASNDGSHLHHPMHPHLSQDPLTGQTILLGPMSTLVHADQISTPETPLPSPPQGSGQEQYKIAANQASVISHQHLSKQKQL

SEQ ID NO: 5 - protein, 11 aa, Synthetic peptide
MKKGHHHHHHG

(19) United States
(12) Patent Application Publication
(10) Pub. No.: US 2005/0131209 A1
(43) Pub. Date: Jun. 16, 2005

(54) Crystallized hnf4 gamma ligand binding domain polypeptide and screening methods employing same

(76) Inventors: Roderick Gerald Davis, Durham, NC (US); Robert Lloyd Johnson, Durham, NC (US); Aaren Bayne Miller, Durham, NC (US); James M Way, Durham, NC (US); Shawn P Williams, Durham, NC (US); Timothy Mark Willson, Durham, NC (US); George Bruce Wisely, Durham, NC (US); Ann Miller Kontz, Wilmington, NC (US)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/470,958

(22) PCT Filed: Jan. 31, 2002
(86) PCT No.: PCT/US02/02992

Related U.S. Application Data
(60) Provisional application No. 60/265,656, filed on Jan. 31, 2001.

(51) Int. Cl. C07K 14/72; G06F 19/00; G01N 33/48; G01N 33/50

(52) U.S. Cl. 530/350; 702/19

(57) ABSTRACT

A solved three-dimensional crystal structure of an HNF4g ligand binding domain polypeptide is disclosed, along with a crystal form of the HNF4g ligand binding domain. Methods of designing modulators of the biological activity of HNF4g, and other HNF4 ligand binding domain polypeptides are also disclosed.

### TECHNICAL FIELD

The present invention relates generally to the structure of the ligand binding domain of HNF4γ, and more particularly to the crystalline structure of the ligand binding domain of HNF4γ. The invention further relates to methods by which modulators and ligands of HNF4γ, and HNF4α, can be identified.

Abbreviations

| | |
|---|---|
| ATP | adenosine triphosphate |
| ADP | adenosine diphosphate |
| APS | Advanced Photon Source |
| BSA | bovine serum albumin |
| CBP | CREB-binding protein |
| cDNA | complementary DNA |
| CI | chemical ionization |
| DBD | DNA binding domain |
| DMSO | dimethyl sulfoxide |
| DNA | deoxyribonucleic acid |
| DTT | dithiothreitol |
| EDTA | ethylenediaminetetraacetic acid |
| EI | electron impact ionization |
| ER | estrogen receptor |
| FAME | fatty acid methyl ester |
| FRET | fluorescent resonance energy transfer |
| GC | gas chromatography |
| GC/MS | gas chromatography/mass spectrometry |
| HEPES | N-2-Hydroxyethylpiperazine-N′-2-ethanesulfonic |
| | acid |
| HNF | hepatocyte nuclear factor |
| HNF1 | hepatocyte nuclear factor 1 |
| HNF4α | hepatocyte nuclear factor 4 α |
| HNF4γ | hepatocyte nuclear factor 4 γ |
| HRE | hormone response element |
| kDa | kilodalton(s) |
| LBD | ligand binding domain |
| MODY | mature onset diabetes of the young |
| MS | mass spectrometry |
| m/z | mass to charge ratio |
| NDP | nucleotide diphosphate |
| nt | nucleotide |
| NTP | nucleotide triphosphate |
| PAGE | polyacrylamide gel electrophoresis |
| PCR | polymerase chain reaction |
| pI | isoelectric point |
| PR | progesterone receptor |
| RAR | retinoic acid receptor |
| RXR | retinoid X receptor |
| SDS | sodium dodecyl sulfate |
| SDS-PAGE | sodium dodecyl sulfate polyacrylamide gel |
| | electrophoresis |
| SIRAS | single isomorphous replacement anomalous |
| | scattering |
| TIC | total ion chromatogram |
| TR | thyroid hormone receptor |
| TTR | plasma transthyretin |
| vHNF | variant hepatocyte nuclear factor |

Amino Acid Abbreviations

| | | |
|---|---|---|
| Single-Letter Code | Three-Letter Code | Name |

| | | |
|---|---|---|
| A | Ala | Alanine |
| V | Val | Valine |
| L | Leu | Leucine |
| I | Ile | Isoleucine |
| P | Pro | Proline |
| F | Phe | Phenylalanine |
| W | Trp | Tryptophan |
| M | Met | Methionine |
| G | Gly | Glycine |
| S | Ser | Serine |
| T | Thr | Threonine |
| C | Cys | Cysteine |
| Y | Tyr | Tyrosine |
| N | Asn | Asparagine |
| Q | Gln | Glutamine |
| D | Asp | Aspartic Acid |
| E | Glu | Glutamic Acid |
| K | Lys | Lysine |
| R | Arg | Arginine |
| H | His | Histidine |

Functionally Equivalent Codons

| | |
|---|---|
| Amino Acid | Codons |

| | | | |
|---|---|---|---|
| Alanine | Ala | A | GCA GCC GCG GCU |
| Cysteine | Cys | C | UGC UGU |
| Aspartic Acid | Asp | D | GAC GAU |
| Glumatic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Methionine | Met | M | AUG |
| Asparagine | Asn | N | AAC AAU |
| Proline | Pro | P | CCA CCC CCG CCU |
| Glutamine | Gln | Q | CAA CAG |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |
| Leucine | Leu | L | UUA UUG CUA CUC |
| | | | CUG CUU |
| Arginine | Arg | R | AGA AGG CGA CGC |
| | | | CGG CGU |
| Serine | Ser | S | ACG AGU UCA UCC |
| | | | UCG UCU |

### BACKGROUND ART

Nuclear receptors represent a superfamily of proteins that specifically bind a physiologically relevant small molecule, such as a hormone or vitamin. As a result of a molecule binding to a nuclear receptor, the nuclear receptor changes the ability of a cell to transcribe DNA, i.e. nuclear receptors modulate the transcription of DNA. However they can also have transcription independent actions.

Unlike integral membrane receptors and membrane-associated receptors, nuclear receptors reside in either the cytoplasm or nucleus of eukaryotic cells. Thus nuclear receptors comprise a class of intracellular, soluble ligand-regulated transcription factors. Nuclear receptors include but are not limited to receptors for glucocorticoids, androgens, mineralcorticoids, progestins, estrogens, thyroid hormones, vitamin D retinoids, icosanoids and peroxisomes. Many nuclear receptors, identified by either sequence homology to known receptors (See, Drewes et al., (1996) Mol. Cell. Biol. 16:925-31) or based on their affinity for specific DNA binding sites in gene promoters (See, Sladek et al., Genes Dev. 4:2353-65), have unascertained ligands and are therefore termed “orphan receptors”.

Hepatocyte Nuclear Factor 4 (HNF4) is an orphan nuclear receptor and two isoforms, HNF4α and HNF4γ, have currently been identified. HNF4α was originally identified based on its ability to bind promoter regions in the plasma transthyretin (TTR) and apoCIII genes. Sladek et al., Genes Dev. 4:2353-65. HNF4γ was identified based on its known homology to HNF4α. Drewes et al., (1996) Mol. Cell. Biol. 16:925-31. Nuclear receptors activate or repress transcription through partner proteins called co-activators or co-repressors, respectively. CREB-binding protein, or CBP, is a known co-activator for HNF4α (Wang et al., (1998) J. Biol. Chem. 273: 30847-50; Dell & Hadzopoulou-Cladaras, (1999) J. Biol. Chem. 274: 9013-21). Mutations in HNF4α have been linked to the metabolic disorder Mature Onset of Diabetes of the Young (MODY), type 1. Yamagata et al., (1996) Nature 384:458-60. HNF4α^{+/-} subjects experience reduced serum levels of apolipoprotein AII, apolipoprotein CIII and lipoproitein(a), leading to reduced triglycerides. Shih et al., (2000) Diabetes 49:832-37. HNF4α regulation had previously been identified for these apolipoprotien genes (Mietus-Snyder et al., (1992) Mol. Cell. Biol. 12:1708-18; Chan et al., (1993) Nucleic Acid Res. 21:1205-11), as well as regulation of other factors involved in glucose metabolism and insulin secretion. Diaz Guerra et al., (1993) Mol. Cell. Biol. 13:7725-33; Miguerol et al., (1994) J. Biol. Chem. 269:8944-51; Stoffel & Duncan, (1997) Proc. Natl. Acad. Sci. U.S.A. 94:13209-14; Wang et al., (1998) J. Biol. Chem. 273:30847-50.

Structurally, the HNF4 family of nuclear receptors, including HNF4α and HNF4γ, are generally characterized by two distinct structural elements. First, nuclear receptors comprise a central DNA binding domain which targets the receptor to specific DNA sequences, which are known as hormone response elements (HREs). The DNA binding domains of these receptors are related in structure and sequence, and are located within the middle of the receptor. Second, the C-terminal region of the HNF4 family of nuclear receptors encompasses the ligand binding domain (LBD). Upon binding a ligand, the receptor shifts to a transcriptionally active state.

Almost all nuclear hormone receptors bind DNA, and the physiologically active complex of many is as a heterodimer with the retinoid X receptor (RXR). The HNF4 isoforms are unusual in that they are obligate homodimers and cannot dimerize with any other nuclear receptors. In fact, retinoid X receptor (RXR) heterodimer formation is actually prevented by LBD interactions. Jiang & Sladek, (1997) J. Biol. Chem. 272:1218-25.

Polypeptides, including the ligand binding domain of HNF4γ, have a three-dimensional structure determined by the primary amino acid sequence and the environment surrounding the polypeptide. This three-dimensional structure establishes the polypeptide's activity, stability, binding affinity, binding specificity, and other biochemical attributes. Thus, knowledge of a protein's three-dimensional structure can provide much guidance in designing agents that mimic, inhibit, or improve its biological activity in soluble or membrane bound forms.

The three-dimensional structure of a polypeptide can be determined in a number of ways. Many of the most precise methods employ X-ray crystallography (See, e.g., Van Holde, (1971) Physical Biochemistry, Prentice-Hall, N.J., 221-39). This technique relies on the ability of crystalline lattices to diffract X-rays or other forms of radiation. Diffraction experiments suitable for determining the three-dimensional structure of macromolecules typically require high-quality crystals. Unfortunately, such crystals have been unavailable for the ligand binding domain of HNF4γ, as well as many other proteins of interest. Thus, high-quality diffracting crystals of the ligand binding domain of HNF4γ would greatly assist in the elucidation of HNF4γ's three-dimensional structure, and would provide insight into the ligand binding properties of HNF4γ.

Clearly, the solved crystal structure of the HNF4γ ligand binding domain would be useful in the design of modulators of activity mediated by all HNF4 isoforms. Evaluation of the available sequence data has made it clear that HNF4α shares significant sequence homology with HNF4γ. Further, HNF4γ shows structural homology with the three-dimensional fold of other proteins.

The solved HNF4γ-ligand crystal structure would provide structural details and insights necessary to design a modulator of HNF4γ that maximizes preferred requirements for any modulator, i.e. potency and specificity. By exploiting the structural details obtained from an HNF4γ-ligand crystal structure, it would be possible to design an HNF4 modulator that, despite HNF4γ's similarity with other proteins, exploits the unique structural features of HNF4γ. An HNF4 modulator developed using structure-assisted design would take advantage of heretofore unknown HNF4 structural considerations and thus be more effective than a modulator developed using homology-based design. Potential or existent homology models cannot provide the necessary degree of specificity. An HNF4γ modulator designed using the structural coordinates of a crystalline form of HNF4γ would also provide a starting point for the development of modulators of other HNF4s.

What is needed, therefore, is a crystallized form of an HNF4γ LBD polypeptide, preferably in complex with a ligand. Acquisition of crystals of the HNF4γ LBD polypeptide will permit the three dimensional structure of the HNF4γ LBD to be determined. Knowledge of this three dimensional structure will facilitate the design of modulators of HNF4γ activity. Such modulators can lead to therapeutic compounds to treat a wide range of conditions, including lipid homeostasis disorders and glucose homeostasis disorders.

### SUMMARY OF THE INVENTION

A substantially pure HNF4γ ligand binding domain polypeptide in crystalline form is disclosed. Preferably, the crystalline form has lattice constants of a=152.71 Å, b=152.71 Å, c=93.42 Å, α=90°, β=90°, γ=90°. More preferably, the crystalline form is a tetragonal crystalline form. Even more preferably, the crystalline form has a space group of 14₁22. Still more preferably, the HNF4γ ligand binding domain polypeptide has the amino acid sequence shown in SEQ ID NO:4.

In a preferred embodiment, the HNF4γ ligand binding domain polypeptide is in complex with a ligand. More preferably, the ligand is a fatty acid.

A method for determining the three-dimensional structure of a crystallized HNF4γ ligand binding domain polypeptide to a resolution of about 3.0 Å or better is also disclosed. The method comprises (a) crystallizing an HNF4γ ligand binding domain polypeptide; and (b) analyzing the HNF4γ ligand binding domain polypeptide to determine the three-dimensional structure of the crystallized HNF4γ ligand binding domain polypeptide, whereby the three-dimensional structure of a crystallized HNF4γ ligand binding domain polypeptide is determined to a resolution of about 3.0 Å or better.

A method of designing a modulator of an HNF4 polypeptide is also disclosed. The method comprises (a) designing a potential modulator of an HNF4 polypeptide that will form bonds with amino acids in a substrate binding site based upon a crystalline structure of an HNF4γ ligand binding domain polypeptide; (b) synthesizing the modulator; and (c) determining whether the potential modulator modulates the activity of the HNF4 polypeptide, whereby a modulator of an HNF4 polypeptide is designed.

In an alternative embodiment, a method of designing a modulator that selectively modulates the activity of an HNF4 polypeptide in accordance with the present invention comprises: (a) obtaining a crystalline form of an HNF4γ ligand binding domain polypeptide; (b) evaluating the three-dimensional structure of the crystallized HNF4γ ligand binding domain polypeptide; and (c) synthesizing a potential modulator based on the three-dimensional crystal structure of the crystallized HNF4γ ligand binding domain polypeptide, whereby a modulator that selectively modulates the activity of an HNF4 polypeptide is designed. Preferably, the method further comprises contacting an HNF4γ ligand binding domain polypeptide with the potential modulator; and assaying the HNF4γ ligand binding domain polypeptide for binding of the potential modulator, for a change in activity of the HNF4γ ligand binding domain polypeptide, or both. More preferably, the crystalline form is such that the three-dimensional structure of the crystallized HNF4γ ligand binding domain polypeptide can be determined to a resolution of about 3.0 Å or better.

In yet another embodiment, a method of designing a modulator of an HNF4 polypeptide in accordance with the present invention comprises: (a) selecting a candidate HNF4 ligand; (b) determining which amino acid or amino acids of an HNF4 polypeptide interact with the ligand using a three-dimensional model of a crystallized protein comprising an HNF4γ LBD; (c) identifying in a biological assay for HNF4 activity a degree to which the ligand modulates the activity of the HNF4 polypeptide; (d) selecting a chemical modification of the ligand wherein the interaction between the amino acids of the HNF4 polypeptide and the ligand is predicted to be modulated by the chemical modification; (e) performing the chemical modification on the ligand to form a modified ligand; (f) contacting the modified ligand with the HNF4 polypeptide; (g) identifying in a biological assay for HNF4 activity a degree to which the modified ligand modulates the biological activity of the HNF4 polypeptide; and (h) comparing the biological activity of the HNF4 polypeptide in the presence of modified ligand with the biological activity of the HNF4 polypeptide in the presence of the unmodified ligand, whereby a modulator of an HNF4 polypeptide is designed. Preferably, the HNF4 polypeptide is an HNF4γ polypeptide. More preferably, the three-dimensional model of a crystallized protein is an HNF4γ LBD polypeptide with a bound ligand. Even more preferably, the method further comprises repeating steps (a) through (f), if the biological activity of the HNF4 polypeptide in the presence of the modified ligand varies from the biological activity of the HNF4 polypeptide in the presence of the unmodified ligand.

A method for identifying an HNF4 modulator is also disclosed. The method comprises (a) providing atomic coordinates of an HNF4γ ligand binding domain to a computerized modeling system; and (b) modeling ligands that fit spatially into the binding pocket of the HNF4γ ligand binding domain to thereby identify an HNF4 modulator. Preferably, the method further comprises identifying in an assay for HNF4-mediated activity a modeled ligand that increases or decreases the activity of the HNF4.

A method of identifying an HNF4γ modulator that selectively modulates the activity of an HNF4γ polypeptide compared to other polypeptides is disclosed. The method comprises (a) providing atomic coordinates of an HNF4γ ligand binding domain to a computerized modeling system; and (b) modeling a ligand that fits into the binding pocket of an HNF4γ ligand binding domain and that interacts with conformationally constrained residues of an HNF4γ that are conserved among HNF4 isoforms to thereby identify an HNF4γ modulator. Preferably, the method further comprises identifying in a biological assay for HNF4γ-mediated activity a modeled ligand that selectively binds to the HNF4γ ligand binding domain and increases or decreases the activity of the HNF4γ.

An assay method for identifying a compound that inhibits binding of a ligand to an HNF4 polypeptide is disclosed. The assay method comprises (a) incubating an HNF4 polypeptide with a ligand in the presence of a test inhibitor compound; (b) determining an amount of ligand that is bound to the HNF4 polypeptide, wherein decreased binding of ligand to the HNF4 protein in the presence of the test inhibitor compound relative to binding of ligand in the absence of the test inhibitor compound is indicative of inhibition; and (c) identifying the test compound as an inhibitor of ligand binding if decreased ligand binding is observed. Preferably, the ligand is a fatty acid.

Accordingly, it is an object of the present invention to provide a three dimensional structure of the ligand binding domain of HNF4γ. The object is achieved in whole or in part by the present invention.

An object of the invention having been stated hereinabove, other objects will be evident as the description proceeds, when taken in connection with the accompanying Drawings and Laboratory Examples as best described hereinbelow.

### BRIEF DESCRIPTION OF THE DRAWINGS

FIG. 1A is a ribbon diagram depicting the structure of the HNF4γ LBD complexed with a natural ligand, palmitic acid. The palmitic acid is depicted in space-filling form. The protein is in gray and palmitic acid is in black, with the oxygen atoms in white.

FIG. 1B is a composite-omit electron density map of the binding pocket of HNF4γ contoured at 1.2σ showing electron density from bound ligand. HNF4γ atoms are shown as a gray ball-and-stick figure.

FIG. 2 is a diagram depicting the binding pocket of the HNF4γ LBD. Palmitic acid, a natural ligand of HNF4γ, is depicted space filling form. Side chains of residues R186, Q145, 1202, A215, V214, M301, M142 and 1305 are involved in ligand binding and are depicted in ball-and-stick form. The protein is in gray and palmitic acid is in black, with the oxygen atoms in white.

FIG. 3 is a bar graph depicting the results of FRET assays performed to detect CBP peptide recruitment.

FIG. 4 is a GC/MS chromatogram of the HNF4γ extract obtained by employing chemical ionization.

FIG. 5 is a chemical ionization mass spectrum for peak g, depicted in FIG. 4. The protonated ion at m/z 271 was subsequently identified as the methyl ester of palmitic acid.

FIG. 6 is a GC/MS chromatogram of the HNF4γ extract obtained by employing electron impact ionization.

FIG. 7 is an electron impact ionization mass spectrum for peak c, depicted in FIG. 6. The molecular ion at m/z 270 was subsequently identified as the methyl ester of palmitic acid.

### DETAILED DESCRIPTION OF THE INVENTION

Until disclosure of the present invention presented herein, the ability to obtain crystalline forms of an HNF4γ LBD has not been realized. And until disclosure of the present invention presented herein, a detailed three-dimensional crystal structure of an HNF4γ polypeptide has not been solved.

In addition to providing structural information, crystalline polypeptides provide other advantages. For example, the crystallization process itself further purifies the polypeptide, and satisfies one of the classical criteria for homogeneity. In fact, crystallization frequently provides unparalleled purification quality, removing impurities that are not removed by other purification methods such as HPLC, dialysis, conventional column chromatography, etc. Moreover, crystalline polypeptides are often stable at ambient temperatures and free of protease contamination and other degradation associated with solution storage. Crystalline polypeptides can also be useful as pharmaceutical preparations. Finally, crystallization techniques in general are largely free of problems such as denaturation associated with other stabilization methods (e.g., lyophilization). Once crystallization has been accomplished, crystallographic data provides useful structural information that can assist the design of compounds that can serve as agonists or antagonists, as described herein below. In addition, the crystal structure provides information useful to map a receptor binding domain, which could then be mimicked by a small non-peptide molecule that would serve as an antagonist or agonist.

I. Definitions

Following long-standing patent law convention, the terms “a” and “an” mean “one or more” when used in this application, including the claims.

As used herein, the term “mutation” carries its traditional connotation and means a change, inherited, naturally occurring or introduced, in a nucleic acid or polypeptide sequence, and is used in its sense as generally known to those of skill in the art.

As used herein, the term “labeled” means the attachment of a moiety, capable of detection by spectroscopic, radiologic or other methods, to a probe molecule.

As used herein, the term “target cell” refers to a cell, into which it is desired to insert a nucleic acid sequence or polypeptide, or to otherwise effect a modification from conditions known to be standard in the unmodified cell. A nucleic acid sequence introduced into a target cell can be of variable length. Additionally, a nucleic acid sequence can enter a target cell as a component of a plasmid or other vector or as a naked sequence.

As used herein, the term “transcription” means a cellular process involving the interaction of an RNA polymerase with a gene that directs the expression as RNA of the structural information present in the coding sequences of the gene. The process includes, but is not limited to the following steps: (a) the transcription initiation, (b) transcript elongation, (c) transcript splicing, (d) transcript capping, (e) transcript termination, (f) transcript polyadenylation, (g) nuclear export of the transcript, (h) transcript editing, and (i) stabilizing the transcript.

As used herein, the term “expression” generally refers to the cellular processes by which a polypeptide is produced from RNA.

As used herein, the term “transcription factor” means a cytoplasmic or nuclear protein which binds to a gene, or binds to an RNA transcript of a gene, or binds to another protein which binds to a gene or an RNA transcript or another protein which in turn binds to a gene or an RNA transcript, so as to thereby modulate expression of the gene. Such modulation can additionally be achieved by other mechanisms; the essence of “transcription factor for a gene” is that the level of transcription of the gene is altered in some way.

As used herein, the term “hybridization” means the binding of a probe molecule, a molecule to which a detectable moiety has been bound, to a target sample.

As used herein, the term “detecting” means confirming the presence of a target entity by observing the occurrence of a detectable signal, such as a radiologic or spectroscopic signal that will appear exclusively in the presence of the target entity.

As used herein, the term “sequencing” means determining the ordered linear sequence of nucleic acids or amino acids of a DNA or protein target sample, using conventional manual or automated laboratory techniques.

As used herein, the term “isolated” means oligonucleotides substantially free of other nucleic acids, proteins, lipids, carbohydrates or other materials with which they can be associated, such association being either in cellular material or in a synthesis medium. The term can also be applied to polypeptides, in which case the polypeptide will be substantially free of nucleic acids, carbohydrates, lipids and other undesired polypeptides.

As used herein, the term “substantially pure” means that the polynucleotide or polypeptide is substantially free of the sequences and molecules with which it is associated in its natural state, and those molecules used in the isolation procedure. The term “substantially free” means that the sample is at least 50%, preferably at least 70%, more preferably 80% and most preferably 90% free of the materials and compounds with which is it associated in nature.

As used herein, the term “primer” means a sequence comprising two or more deoxyribonucleotides or ribonucleotides, preferably more than three, and more preferably more than eight and most preferably at least about 20 nucleotides of an exonic or intronic region. Such oligonucleotides are preferably between ten and thirty bases in length.

As used herein, the term “DNA segment” means a DNA molecule that has been isolated free of total genomic DNA of a particular species. In a preferred embodiment, a DNA segment encoding an HNF4 polypeptide refers to a DNA segment that contains SEQ ID NO:1, but can optionally comprise fewer or additional nucleic acids, yet is isolated away from, or purified free from, total genomic DNA of a source species, such as Homo sapiens. Included within the term “DNA segment” are DNA segments and smaller fragments of such segments, and also recombinant vectors, including, for example, plasmids, cosmids, phages, viruses, and the like.

As used herein, the phrase “enhancer-promoter” means a composite unit that contains both enhancer and promoter elements. An enhancer-promoter is operatively linked to a coding sequence that encodes at least one gene product.

As used herein, the phrase “operatively linked” means that an enhancer-promoter is connected to a coding sequence in such a way that the transcription of that coding sequence is controlled and regulated by that enhancer-promoter. Techniques for operatively linking an enhancer-promoter to a coding sequence are well known in the art; the precise orientation and location relative to a coding sequence of interest is dependent, inter alia, upon the specific nature of the enhancer-promoter.

As used herein, the terms “candidate substance” and “candidate compound” are used interchangeably and refer to a substance that is believed to interact with another moiety, for example a given ligand that is believed to interact with a complete, or a fragment of, an HNF4 polypeptide, and which can be subsequently evaluated for such an interaction. Representative candidate substances or compounds include xenobiotics such as drugs and other therapeutic agents, carcinogens and environmental pollutants, natural products and extracts, as well as endobiotics such as steroids, fatty acids and prostaglandins. Other examples of candidate compounds that can be investigated using the methods of the present invention include, but are not restricted to, agonists and antagonists of an HNF4 polypeptide, toxins and venoms, viral epitopes, hormones (e.g., opioid peptides, steroids, etc.), hormone receptors, peptides, enzymes, enzyme substrates, co-factors, lectins, sugars, oligonucleotides or nucleic acids, oligosaccharides, proteins, small molecules and monoclonal antibodies.

As used herein, the term “biological activity” means any observable effect flowing from interaction between an HNF4 polypeptide and a ligand. Representative, but non-limiting, examples of biological activity in the context of the present invention include homodimerization of an HNF4, lipid binding by HNF4 and association of an HNF4 with DNA.

As used herein, the term “modified” means an alteration from an entity's normally occurring state. An entity can be modified by removing discrete chemical units or by adding discrete chemical units. The term “modified” encompasses detectable labels as well as those entities added as aids in purification.

As used herein, the terms “structure coordinates” and “structural coordinates” mean mathematical coordinates derived from mathematical equations related to the patterns obtained on diffraction of a monochromatic beam of X-rays by the atoms (scattering centers) of a molecule in crystal form. The diffraction data are used to calculate an electron density map of the repeating unit of the crystal. The electron density maps are used to establish the positions of the individual atoms within the unit cell of the crystal.

Those of skill in the art understand that a set of structure coordinates determined by X-ray crystallography is not without standard error. For the purpose of this invention, any set of structure coordinates for HNF4γ or an HNF4γ mutant that have a root mean square (RMS) deviation from ideal of no more than 0.5 Å when superimposed, using the polypeptide backbone atoms, on the structure coordinates listed in Table 2 shall be considered identical.

As used herein, the term “space group” means the arrangement of symmetry elements of a crystal.

As used herein, the term “molecular replacement” means a method that involves generating a preliminary model of the wild-type HNF4γ ligand binding domain, or an HNF4γ mutant crystal whose structure coordinates are unknown, by orienting and positioning a molecule whose structure coordinates are known within the unit cell of the unknown crystal so as best to account for the observed diffraction pattern of the unknown crystal. Phases can then be calculated from this model and combined with the observed amplitudes to give an approximate Fourier synthesis of the structure whose coordinates are unknown. This, in turn, can be subject to any of the several forms of refinement to provide a final, accurate structure of the unknown crystal. See, e.g., Lattman, (1985) Method Enzymol., 115: 55-77; Rossmann, ed, (1972) The Molecular Replacement Method, Gordon & Breach, New York.) Using the structure coordinates of the ligand binding domain of HNF4γ provided by this invention, molecular replacement can be used to determine the structure coordinates of a crystalline mutant or homologue of the HNF4γ ligand binding domain, or of a different crystal form of the HNF4γ ligand binding domain.

As used herein, the term “isomorphous replacement” means a method of using heavy atom derivative crystals to obtain the phase information necessary to elucidate the three-dimensional structure of a native crystal (Blundell et al., (1976) Protein Crystallography, Academic Press; Otwinowski, (1991), in Isomorphous Replacement and Anomalous Scattering, (Evans & Leslie, eds.), pp. 80-86, Daresbury Laboratory, Daresbury, United Kingdom). The phrase “heavy-atom derivatization” is synonymous with the term “isomorphous replacement”.

As used herein, the terms “β-sheet” and “beta-sheet” mean the conformation of a polypeptide chain stretched into an extended zig-zig conformation. Portions of polypeptide chains that run “parallel” all run in the same direction. Polypeptide chains that are “antiparallel” run in the opposite direction from the parallel chains.

As used herein, the terms “α-helix” and “alpha-helix” mean the conformation of a polypeptide chain wherein the polypeptide backbone is wound around the long axis of the molecule in a left-handed or right-handed direction, and the R groups of the amino acids protrude outward from the helical backbone, wherein the repeating unit of the structure is a single turnoff the helix, which extends about 0.56 nm along the long axis.

As used herein, the term “unit cell” means a basic parallelepiped shaped block. The entire volume of a crystal can be constructed by regular assembly of such blocks. Each unit cell comprises a complete representation of the unit of pattern, the repetition of which builds up the crystal. Thus, the term “unit cell” means the fundamental portion of a crystal structure that is repeated infinitely by translation in three dimensions. A unit cell is characterized by three vectors a, b, and c, not located in one plane, which form the edges of a parallelepiped. Angles α, β and γ define the angles between the vectors: angle α is the angle between vectors b and c; angle β is the angle between vectors a and c; and angle γ is the angle between vectors a and b. The entire volume of a crystal can be constructed by regular assembly of unit cells; each unit cell comprises a complete representation of the unit of pattern, the repetition of which builds up the crystal.

As used herein, the term “tetragonal unit cell” means a unit cell wherein a=b≠c; and α=β=γ=90°. The vectors a, b and c describe the unit cell edges and the angles α, β, and γ describe the unit cell angles.

As used herein, the term “crystal lattice” means the array of points defined by the vertices of packed unit cells.

As used herein, the term “active site” means that site in a polypeptide where substrate binding occurs. For HNF4γ, the active site comprises the residues Ile135, Val138, Cys139, Ser141, Met142, Gln145, Leu179, Leu180, Gly182, Ala183, Arg186, Leu194, Leu196, Gly197, Ile202, Glu210, Ile211, Val214, Ala215, Val218, Met301, Gln304, Ile305, Val308, Val314, Ile316 and Leu320.

As used herein, the term “HNF4” means nucleic acids encoding a hepatocyte nuclear factor 4 (HNF4) nuclear receptor polypeptide that can bind DNA and/or one or more ligands and/or has the ability to form multimers. The term “HNF4” encompasses at least the HNF4α and HNF4γ isoforms. The term “HNF4” includes invertebrate homologs; however, preferably, HNF4 nucleic acids and polypeptides are isolated from vertebrate sources. “HNF4” further includes vertebrate homologs of HNF4 family members, including, but not limited to, mammalian and avian homologs. Representative mammalian homologs of HNF4 family members include, but are not limited to, murine and human homologs.

As used herein, the terms “HNF4 gene product”, “HNF4 protein”, “HNF4 polypeptide”, and “HNF4 peptide” are used interchangeably and mean peptides having amino acid sequences which are substantially identical to native amino acid sequences from an organism of interest and which are biologically active in that they comprise all or a part of the amino acid sequence of an HNF4 polypeptide, or cross-react with antibodies raised against an HNF4 polypeptide, or retain all or some of the biological activity (e.g., DNA or ligand binding ability and/or dimerization ability) of the native amino acid sequence or protein. Such biological activity can include immunogenicity.

As used herein, the terms “HNF4 gene product”, “HNF4 protein”, “HNF4 polypeptide”, and “HNF4 peptide” also include analogs of an HNF4 polypeptide. By “analog” is intended that a DNA or peptide sequence can contain alterations relative to the sequences disclosed herein, yet retain all or some of the biological activity of those sequences. Analogs can be derived from genomic nucleotide sequences as are disclosed herein or from other organisms, or can be created synthetically. Those skilled in the art will appreciate that other analogs, as yet undisclosed or undiscovered, can be used to design and/or construct HNF4 analogs. There is no need for an “HNF4 gene product”, “HNF4 protein”, “HNF4 polypeptide”, or “HNF4 peptide” to comprise all or substantially all of the amino acid sequence of an HNF4 polypeptide gene product. Shorter or longer sequences are anticipated to be of use in the invention; shorter sequences are herein referred to as “segments”. Thus, the terms “HNF4 gene product”, “HNF4 protein”, “HNF4 polypeptide”, and “HNF4 peptide” also include fusion, chimeric or recombinant HNF4 polypeptides and proteins comprising sequences of the present invention. Methods of preparing such proteins are disclosed herein and are known in the art.

In the present invention, the terms “HNF4γ gene product”, “HNF4γ protein”, “HNF4γ polypeptide”, and “HNF4γ peptide” are used interchangeably and mean to a preferred isoform of an HNF4 polypeptide family, namely HNF4γ. A more preferred embodiment of an HNF4γ polypeptide comprises the amino acid sequence of SEQ ID NO:2.

As used herein, the term “polypeptide” means any polymer comprising any of the 20 protein amino acids, regardless of its size. Although “protein” is often used in reference to relatively large polypeptides, and “peptide” is often used in reference to small polypeptides, usage of these terms in the art overlaps and varies. The term “polypeptide” as used herein refers to peptides, polypeptides and proteins, unless otherwise noted. As used herein, the terms “protein”, “polypeptide” and “peptide” are used interchangeably herein when referring to a gene product.

As used herein, the term “modulate” means an increase, decrease, or other alteration of any or all chemical and biological activities or properties of a wild-type or mutant HNF4 polypeptide, preferably a wild-type or mutant HNF4γ polypeptide. The term “modulation” as used herein refers to both upregulation (i.e., activation or stimulation) and downregulation (i.e. inhibition or suppression) of a response.

As used herein, the term “diabetes” means disorders related to alterations in glucose homeostasis. In the mildest forms of diabetes, this alteration is detected only after challenge with a carbohydrate load, while in moderate to severe forms of disease, hyperglycemia is present. Type I diabetes, insulin dependent diabetes mellitus or IDDM, is the result of a progressive autoimmune destruction of the pancreatic β-cells with subsequent insulin deficiency. The more prevalent Type II, non-insulin dependent diabetes mellitus or NIDDM, is associated with peripheral insulin resistance, elevated hepatic glucose production, and inappropriate insulin secretion. Type II diabetes that develops during the age of 20-30 years old and is associated with chronic hyperglycemia and monogenic inheritance is referred to as maturity onset diabetes of the young (MODY, Type II). Other forms of Type II diabetes develop in an individual sometime after 20-30 years of age, for example, late-onset NIDDM. HNF4α is linked to MODY I.

As used herein, the terms “HNF4 gene” and “recombinant HNF4 gene” mean a nucleic acid molecule comprising an open reading frame encoding an HNF4 polypeptide of the present invention, including both exon and (optionally) intron sequences.

As used herein, the term “gene” is used for simplicity to refer to a functional protein, polypeptide or peptide encoding unit. As will be understood by those in the art, this functional term includes both genomic sequences and cDNA sequences. Preferred embodiments of genomic and cDNA sequences are disclosed herein.

As used herein, the term “DNA sequence encoding an HNF4 polypeptide” can refer to one or more coding sequences within a particular individual. Moreover, certain differences in nucleotide sequences can exist between individual organisms, which are called alleles. It is possible that such allelic differences might or might not result in differences in amino acid sequence of the encoded polypeptide yet still encode a protein with the same biological activity. As is well known, genes for a particular polypeptide can exist in single or multiple copies within the genome of an individual. Such duplicate genes can be identical or can have certain modifications, including nucleotide substitutions, additions or deletions, all of which still code for polypeptides having substantially the same activity.

As used herein, the term “intron” means a DNA sequence present in a given gene which is not translated into protein.

As used herein, the term “interact” means detectable interactions between molecules, such as can be detected using, for example, a yeast two hybrid assay. The term “interact” is also meant to include “binding” interactions between molecules. Interactions can, for example, be protein-protein or protein-nucleic acid in nature.

As used herein, the terms “cells,” “host cells” or “recombinant host cells” are used interchangeably and mean not only to the particular subject cell, but also to the progeny or potential progeny of such a cell. Because certain modifications can occur in succeeding generations due to either mutation or environmental influences, such progeny might not, in fact, be identical to the parent cell, but are still included within the scope of the term as used herein.

As used herein, the term “agonist” means an agent that supplements or potentiates the bioactivity of a functional HNF4 gene or protein or of a polypeptide encoded by a gene that is up- or down-regulated by an HNF4 polypeptide and/or a polypeptide encoded by a gene that contains an HNF4 binding site in its promoter region.

As used herein, the term “antagonist” means an agent that decreases or inhibits the bioactivity of a functional HNF4 gene or protein, or that supplements or potentiates the bioactivity of a naturally occurring or engineered non-functional HNF4 gene or protein. Alternatively, an antagonist can decrease or inhibit the bioactivity of a functional gene or polypeptide encoded by a gene that is up- or down-regulated by an HNF4 polypeptide and/or contains an HNF4 binding site in its promoter region. An antagonist can also supplement or potentiate the bioactivity of a naturally occurring or engineered non-functional gene or polypeptide encoded by a gene that is up- or down-regulated by an HNF4 polypeptide, and/or contains an HNF4 binding site in its promoter region.

As used herein, the terms “chimeric protein” or “fusion protein” are used interchangeably and mean a fusion of a first amino acid sequence encoding an HNF4 polypeptide with a second amino acid sequence defining a polypeptide domain foreign to, and not homologous with, any domain of one of an HNF4 polypeptide. A chimeric protein can present a foreign domain which is found in an organism which also expresses the first protein, or it can be an “interspecies” or “intergenic” fusion of protein structures expressed by different kinds of organisms. In general, a fusion protein can be represented by the general formula X—HNF4—Y, wherein HNF4 represents a portion of the protein which is derived from an HNF4 polypeptide, and X and Y are independently absent or represent amino acid sequences which are not related to an HNF4 sequence in an organism, which includes naturally occurring mutants.

II. Description of Tables

Table 1 is a table summarizing the crystal and data statistics obtained from the crystallized ligand binding domain of HNF4γ. Data on the unit cell are presented, including data on the crystal space group, unit cell dimensions, molecules per asymmetric cell and crystal resolution.

Table 2 is a table of the atomic structure coordinate data obtained from X-ray diffraction from the ligand binding domain of HNF4γ in complex with a ligand.

Table 3 is a table depicting a sequence alignment comparing HNF4γ and HNF4α. Boxed HNF4γ residues are in alpha helices, shaded HNF4γ residues are in beta strands. Bold HNF4γ residues are thoser residues that have the potential to form Van der Waals's contacts (5Å) with palmitic acid; bold and underlined HNF4γ residues form hydrogen bonds to palmitic acid. Underlined HNF4α residues are mutations associated with the disease MODY 1.

Table 4 is a table summarizing data obtained from analytes detected by GC/MS using chemical ionization

III. General Considerations

Hepatocyte nuclear factor cDNAs code for several different genes and map to different chromosomes. HNF1 maps to chromosome 12, vHNF1 maps to chromosome 17, HNF4α maps to chromosome 20 and HNF4γ maps to chromosome 8. HNF1 and vHNF1 are homologous to each other, regulate several of the same genes and have similar tissues expression patterns. HNF4α and HNF4γ are also homologous to each other. Additionally, HNF4α and HNF4γ have an overlapping, but not identical expression pattern. The existence of multiple isoforms of the HNF4 polypeptide could explain the complex forms of regulation controlled by these transcription factors in different tissues. The redundancy of these transcription factors suggests the possibility of biological complementation by these genes, with respect to each other; when one isoform is defective, for example in a subject afflicted with diabetes, the other isoform could compensate.

The present invention will usually be applicable mutatis mutandis to all HNF4 polypeptides, as discussed herein based, in part, on the patterns of HNF4 structure and modulation that have emerged as a consequence of determining the three dimensional structure of HNF4γ in complex with a ligand. Generally, the HNF4s display substantial regions of amino acid homology. Additionally, the HNF4s display an overall structural motif comprising three modular domains:
1) a variable amino-terminal domain;
2) a highly conserved DNA-binding domain (DBD); and
3) a less conserved carboxy-terminal ligand binding domain (LBD).
   The modularity of the HNF4s permits different domains of each protein to separately accomplish different functions, although the domains can influence each other. The separate function of a domain is usually preserved when a particular domain is isolated from the remainder of the protein. Using conventional protein chemistry techniques, a modular domain can sometimes be separated from the parent protein. Using conventional molecular biology techniques, each domain can usually be separately expressed with its original function intact or, as discussed herein below, chimeric proteins comprising two different proteins can be constructed, wherein the chimeric proteins retain the properties of the individual functional domains of the respective polypeptides from which the chimeric proteins were generated.

The amino terminal domain of the HNF4 isoforms is the least conserved of the three domains. This domain is involved in transcriptional activation and, in some cases, its uniqueness can dictate selective receptor-DNA binding and activation of target genes by HNF4 isoforms.

The DNA binding domain is the most conserved structure amongst the HNF4s. It typically contains about 70 amino acids that fold into two zinc finger motifs, wherein a zinc ion coordinates four cysteines. The DBD contains two perpendicularly oriented α-helices that extend from the base of the first and second zinc fingers. The two zinc fingers function in concert along with non-zinc finger residues to direct the HNF4s to specific target sites on DNA. Various amino acids in the DBD influence spacing between two half-sites (which usually comprises six nucleotides) for receptor homodimerization. The optimal spacings facilitate cooperative interactions between DBDs, and D box residues are part of the dimerization interface. Other regions of the DBD facilitate DNA-protein and protein-protein interactions required for HNF4 homodimerization.

The LBD is the second most highly conserved domain in these receptors. Whereas the integrity of several different LBD sub-domains is important for ligand binding, truncated molecules containing only the LBD can retain normal ligand binding activity. This domain also participates in other functions, including dimerization, nuclear translocation and transcriptional regulation activities. Importantly, this domain can bind a ligand and can undergo ligand-induced conformational changes. Ligand binding allows the activation domain to serve as an interaction site for essential co-activator proteins that function to stimulate or inhibit transcription.

The carboxy-terminal activation subdomain is in close three-dimensional proximity in the LBD to the ligand, so as to allow for ligands bound to the LBD to coordinate (or interact) with amino acid(s) in the activation subdomain. As disclosed herein, the LBD of an HNF4 is expressed, crystallized and its three dimensional structure determined. Computational and other methods for the design of ligands to the LBD are also disclosed.

IV. Production of HNF4 Polypeptides

The native and mutated HNF4 polypeptides, and fragments thereof, of the present invention can be chemically synthesized in whole or part using techniques that are well-known in the art (See, e.g., Creighton, (1983) Proteins: Structures and Molecular Principles, W.H. Freeman & Co., New York, incorporated herein in its entirety). Alternatively, methods which are well known to those skilled in the art can be used to construct expression vectors containing a partial or the entire native or mutated HNF4 polypeptide coding sequence and appropriate transcriptional/translational control signals. These methods include in vitro recombinant DNA techniques, synthetic techniques and in vivo recombination/genetic recombination. See, for example, the techniques described in Sambrook et al., (1989) Molecular Cloning: A Laboratory Manual, Cold Spring Harbor Laboratory, New York, and Ausubel et al., (1989) Current Protocols in Molecular Biology, Greene Publishing Associates and Wiley Interscience, New York, both incorporated herein in their entirety.

A variety of host-expression vector systems can be utilized to express an HNF4 coding sequence. These include but are not limited to microorganisms such as bacteria transformed with recombinant bacteriophage DNA, plasmid DNA or cosmid DNA expression vectors containing an HNF4 coding sequence; yeast transformed with recombinant yeast expression vectors containing an HNF4 coding sequence; insect cell systems infected with recombinant virus expression vectors (e.g., baculovirus) containing an HNF4 coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing an HNF4 coding sequence; or animal cell systems. The expression elements of these systems vary in their strength and specificities.

Depending on the host/vector system utilized, any of a number of suitable transcription and translation elements, including constitutive and inducible promoters, can be used in the expression vector. For example, when cloning in bacterial systems, inducible promoters such as pL of bacteriophage λ, plac, ptrp, ptac (ptrp-lac hybrid promoter) and the like can be used. When cloning in insect cell systems, promoters such as the baculovirus polyhedrin promoter can be used. When cloning in plant cell systems, promoters derived from the genome of plant cells, such as heat shock promoters; the promoter for the small subunit of RUBISCO; the promoter for the chlorophyll a/b binding protein) or from plant viruses (e.g., the ³⁵S RNA promoter of CaMV; the coat protein promoter of TMV) can be used. When cloning in mammalian cell systems, promoters derived from the genome of mammalian cells (e.g., metallothionein promoter) or from mammalian viruses (e.g., the adenovirus late promoter; the vaccinia virus 7.5 K promoter) can be used. When generating cell lines that contain multiple copies of the tyrosine kinase domain DNA, SV40-, BPV- and EBV-based vectors can be used with an appropriate selectable marker.

V. Formation of HNF4γ Ligand Binding Domain Crystals

In one embodiment, the present invention provides crystals of HNF4γ. The crystals were obtained using the methodology disclosed in the Examples. The HNF4γ crystals, which can be native crystals, derivative crystals or co-crystals, have tetragonal unit cells (a tetragonal unit cell is a unit cell wherein a=b≠c, and wherein α=β=γ=90°) and space group symmetry 14₁22. There is one HNF4γ molecule in the asymmetric unit. In the HNF4γ crystalline form, the unit cell has dimensions of a=b=152.71 c=93.42, and α=β=γ=90°.

The HNF4γ LBD-ligand structure was solved using single isomorphous replacement anomalous scattering (SIRAS) techniques. In the SIRAS method of solving protein crystals, a derivative crystal is prepared that contains an atom that is heavier than the other atoms of the sample. One representative heavy atom that can be incorporated into the derivative crystal is mercury. A mercury-based heavy atom derivative crystal was used to solve the structure of the HNF4γ ligand binding domain of the present invention. Heavy atom derivative crystals can be prepared by soaking a crystal in a solution containing a selected heavy atom salt. In the present invention, heavy atom derivative crystals were prepared by soaking a crystalline form of the HNF4γ LBD in methyl mercury chloride (MeHgCl).

Symmetry-related reflections in the X-ray diffraction pattern, usually identical, are altered by the anomalous scattering contribution of the heavy atoms. The measured differences in symmetry-related reflections are used to determine the position of the heavy atoms, leading to an initial estimation of the diffraction phases, and subsequently, an electron density map is prepared. The prepared electron density map is then used to identify the position of the other atoms in the sample.

V.A. Preparation of HNF4 Crystals

The native and derivative co-crystals, and fragments thereof, disclosed in the present invention can be obtained by a variety of techniques, including batch, liquid bridge, dialysis, vapor diffusion and hanging drop methods (See, e.g., McPherson, (1982) Preparation and Analysis of Protein Crystals, John Wiley, New York.; McPherson, (1990) Eur. J. Biochem. 189:1-23.; Weber, (1991) Adv. Protein Chem. 41:1-36). In a preferred embodiment, the vapor diffusion and hanging drop methods are used for the crystallization of HNF4 polypeptides and fragments thereof.

In general, native crystals of the present invention are grown by dissolving substantially pure HNF4 polypeptide or a fragment thereof in an aqueous buffer containing a precipitant at a concentration just below that necessary to precipitate the protein. Water is removed by controlled evaporation to produce precipitating conditions, which are maintained until crystal growth ceases.

In a preferred embodiment of the invention, native crystals are grown by vapor diffusion (See, e.g., McPherson, (1982) Preparation and Analysis of Protein Crystals, John Wiley, New York.; McPherson, (1990) Eur. J. Biochem. 189:1-23). In this method, the polypeptide/precipitant solution is allowed to equilibrate in a closed container with a larger aqueous reservoir having a precipitant concentration optimal for producing crystals. Generally, less than about 25 μL of HNF4 polypeptide solution is mixed with an equal volume of reservoir solution, giving a precipitant concentration about half that required for crystallization. This solution is suspended as a droplet underneath a coverslip, which is sealed onto the top of the reservoir. The sealed container is allowed to stand, until crystals grow. Crystals generally form within two to six weeks, and are suitable for data collection within approximately seven to ten weeks. Of course, those of skill in the art will recognize that the above-described crystallization procedures and conditions can be varied.

V.B. Preparation of Derivative Crystals

Derivative crystals of the present invention, e.g. heavy atom derivative crystals, can be obtained by soaking native crystals in mother liquor containing salts of heavy metal atoms. Such derivative crystals are useful for phase analysis in the solution of crystals of the present invention. In a preferred embodiment of the present invention, for example, soaking a native crystal in a solution containing methyl-mercury chloride provides derivative crystals suitable for use as isomorphous replacements in determining the X-ray crystal structure of an HNF4 polypeptide. Additional reagents useful for the preparation of the derivative crystals of the present invention will be apparent to those of skill in the art after review of the disclosure of the present invention presented herein.

V.C. Preparation of Co-Crystals

Co-crystals of the present invention can be obtained by soaking a native crystal in mother liquor containing compounds known or predicted to bind the LBD of an HNF4, or a fragment thereof. Alternatively, co-crystals can be obtained by co-crystallizing an HNF4 LBD polypeptide or a fragment thereof in the presence of one or more compounds known or predicted to bind the polypeptide. In a preferred embodiment, such a compound is a fatty acid of variable length.

V.D. Solving a Crystal Structure of the Present Invention

Crystal structures of the present invention can be solved using a variety of techniques including, but not limited to, isomorphous replacement anomalous scattering or molecular replacement methods. Computer software packages will also be helpful in solving a crystal structure of the present invention. Applicable software packages include but are not limited to X-PLOR™ program (Brünger, (1992) X-PLOR, Version 3.1. A System for X-ray Crystallography and NMR, Yale University Press, New Haven, Conn.; X-PLOR is available from Molecular Simulations, Inc., San Diego, Calif.), Xtal View (McRee, (1992) J. Mol. Graphics 10: 44-47; X-tal View is available from the San Diego Supercomputer Center), SHELXS 97 (Sheldrick (1990) Acta Cryst. A46: 467; SHELX 97 is available from the Institute of Inorganic Chemistry, Georg-August-Universitat, Göttingen, Germany), HEAVY (Terwilliger, Los Alamos National Laboratory) can be used and SHAKE-AND-BAKE (Hauptman, (1997) Curr. Opin. Struct. Biol. 7: 672-80; Weeks et al., (1993) Acta Cryst. D49: 179; available from the Hauptman-Woodward Medical Research Institute, Buffalo, N.Y.). See also, Ducruix & Geige, (1992) Crystallization of Nucleic Acids and Proteins: A Practical Approach, IRL Press, Oxford, England, and references cited therein.

VI. Summary of Results for the HNF4γ Ligand Binding Domain

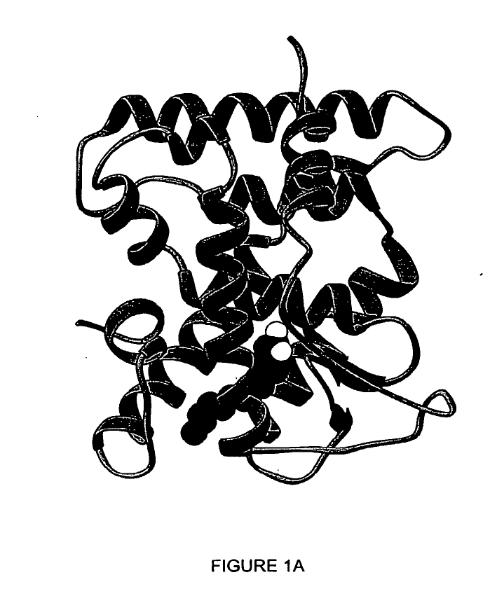

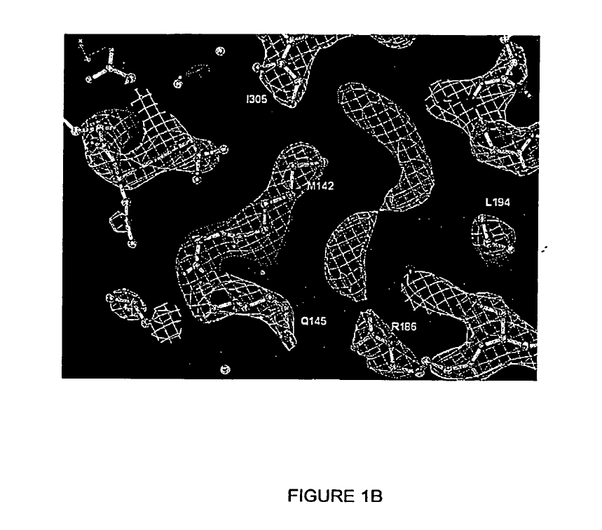

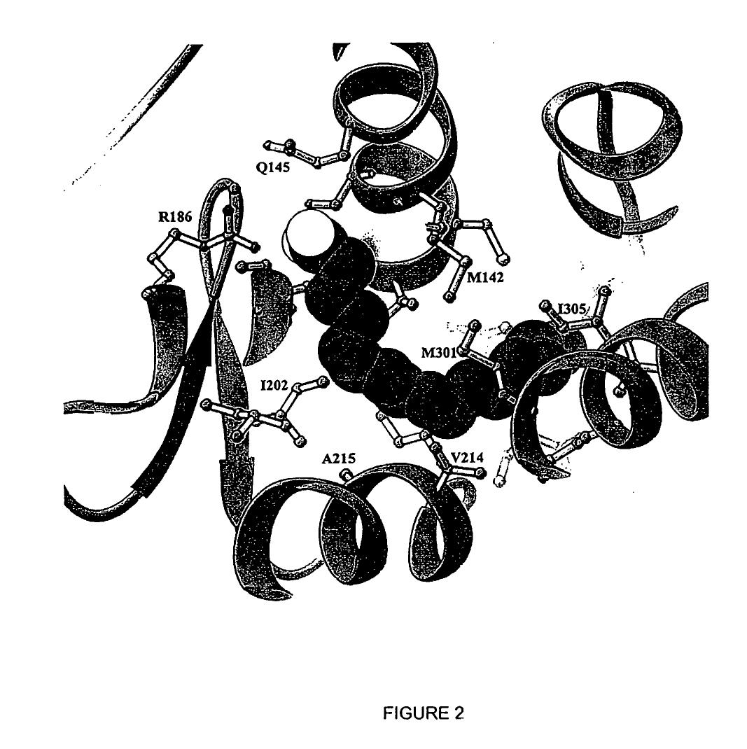

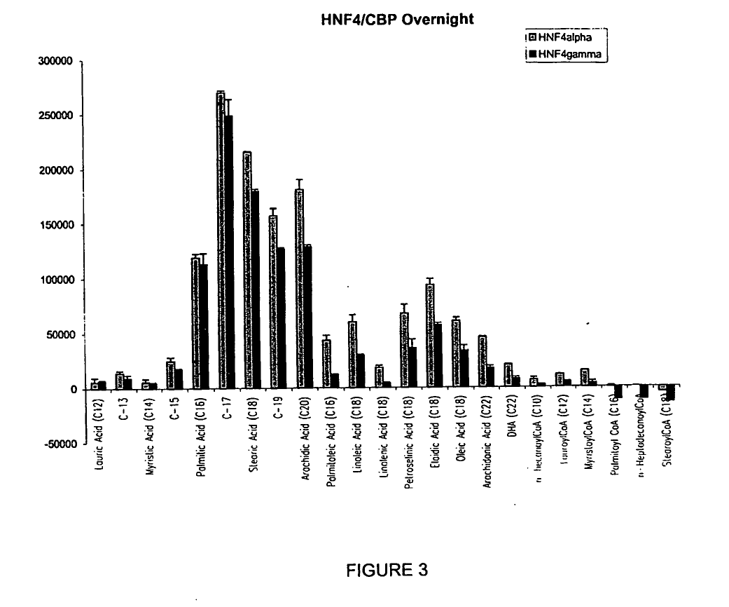

The three-dimensional structure of the HNF4γ LBD has been solved by X-ray crystallography and is depicted in FIG. 1A. The structure of HNF4γ is shown to contain the characteristic ligand binding pocket observed for “classic” nuclear receptors. The ligand binding pocket is depicted in FIG. 2. The long HNF4γ F-domain was present in the crystals but was disordered, suggesting that it did not make strong interactions with the core residues of the LBD. The HNF4γ LBD induces obligate homodimerization due to deviations from the conserved heterodimer motif. The structure of HNF4γ revealed a bound ligand. The identity of this ligand was determined to be the fatty acids palmitic acid and stearic acid by GC/EI/MS. The saturated fatty acids palmitic acid and stearic acid were shown to be functional activators for two HNF4 isoforms, HNF4α and HNF4γ, using a FRET assay to detect CBP peptide recruitment. FIG. 3 depicts the results of the FRET assay. Shorter fatty acids had no effect on CBP peptide recruitment. Fatty acyl CoA derivatives of palmitic acid and stearic acid had a small negative effect on CBP recruitment, suggesting that they were not activators for HNF4α or HNF4γ.

VI.A. Overall Structure of the HNF41 LBD

The overall fold of the HNF4γ LBD of the present invention is an “α-helical sandwich”, is depicted in FIG. 1A, is similar to that observed in other nuclear receptor LBDs, and is most similar to holo-RXRα (Bourguet et al., (2000) Mol. Cell 5: 289-98; Gampe, Jr. et al., (2000) Mol. Cell 5: 545-55). Although mass spectrometry confirmed that crystals contained full length HNF4γ LBD (amino acids 102408), only amino acids 102-118 and 123-327 are visible in the electron density map. These residues comprise the “core” LBD, and contain the conserved structural motifs observed in other nuclear receptor LBD's. The observation that the HNF4γ C-terminal tail is disordered suggests that the strong interactions between the “core” LBD and the C-terminal tail seen in PR and AR are absent in HNF4. The AF2 helix of HNF4γ (amino acids 316-325) is in the “active” conformation, characteristic of other agonist-bound nuclear receptors.

VI.B. Structural Features of the Dimerization Site

The HNF4γ homodimer interface is composed of residues in helices 7, 9 and 10, and is the same interface seen in other nuclear receptor homo- and heterodimers (Bourguet et al., (1995) Nature 375: 377-82; Brzozowski et al., (1997) Nature (London) 389: 753-58; Nolte et al., (1998) Nature (London) 395: 1374, (Bourguet et al., (2000) Mol. Cell 5: 289-98; Gampe, Jr. et al., (2000) Mol. Cell 5: 545-55). Of the 22 residues involved in HNF4γ dimerization, 20 are conserved in HNF4α, and all charged residues in the HNF4γ dimer interface are identical in HNF4α. This homodimer interface exemplifies themes seen in other nuclear receptor dimers, buried hydrophobic surface for stability, with hydrogen bonds and charge-pairing for specificity. As a dimer HNF4γ buries 1320 Å² of accessible surface per monomer, between the 1266 Å² and 1632 Å² observed for RXRα and ERα homodimers, respectively. The HNF4γ homodimer interface includes specific side-chain/side-chain interactions, with hydrogen bonds between Q266Nε-E2860ε and Q295Oε-Q295Nε and salt bridges between E228Oε-K259Nζand possibly D271Oδ-R281Nε, and R281NH. HNF4/RXR heterodimer formation is prevented by LBD interactions (Jiang & Sladek, (1997) J. Biol. Chem. 272: 1218-25), and LBD heterodimer formation is precluded because not all salt-bridges will form. The RXR equivalent to HNF4γ E228 and K259 are D359 and E390, respectively, so a heterodimer will create one salt bridge and one potentially unfavorable pairing. HNF4γ D271 and R281 are equivalent to RXRα A402 and P412, respectively, and no charge-pairing is possible. Also, the critical heterodimer salt bridge observed between RXRα R393 and PPARγ D441 cannot be made to HNF4, where the equivalent residue is a Thr. Our observation that the E228-K259 salt bridge is important for homodimer formation agrees with the work of Bogan et al. (Bogan et al., (2000) J. Mol. Biol. 302: 831-851). Their results showed that wild-type HNF4α cannot form heterodimers with HNF4α mutants where residues E327 (γE286) and K300 (γK259) are changed to their RXR equivalents. The conservation of the interface residues between HNF4γ and HNF4α suggests that the HNF4α homodimer interface is similar to HNF4γ's. In fact, HNF4α/HNF4γ heterodimers could possibly exist in tissues where both are present.

VI.C. Structural Features of the HNF4γ Binding Pocket

The HNF4γ LBD has a well-defined ligand binding pocket, which is similar to the nuclear receptors RXR (Bourguet et al., (1995) Nature 375: 377-82) and RAR (Renaud et al., (1995) Nature 378: 681-89). The pocket volume, 476 Å³, is consistent with binding a small molecule ligand, and is hydrophobic over 76% of the pocket surface. Arginine 186, which is conserved among a number of nuclear receptors, occupies the same pocket position seen in retinoid X receptor (RXR), retinoic acid receptor (RAR), thyroid hormone receptor (TR), estrogen receptor (ER) and progesterone receptor (PR). In all previous structures, this binding-pocket arginine makes hydrogen bonds to oxygen atoms of bound ligands. HNF4γ's pocket is too narrow to accommodate steroids. Another prominent feature of the pocket is a direct contact between M142 in helix 3 and M301 in helix 11. This contact bridges the binding pocket, and effectively blocks direct ligand access to residues 318-325 in helix 12.

Palmitic acid forms hydrogen bonds with the side chain of arginine 186, and with the backbone nitrogen of glycine 197 (not shown). Alanine 215 corresponds to serine 256 in HNF4α. Because serine can form hydrogen bonds to the ligand, the specificity is different for the two receptor subtypes. GC/MS analyses of receptor extracts indicates that although HNF4α also binds palmitic and stearic acid, it preferentially binds different fatty acids. Valine 214 corresponds to valine 255 in HNF4α, and is one of the mutations associated with MODY, Type 1.

VI.D. Identification and Characterization of an HNF4α Binding Pocket Ligand

Electron density was observed in the HNF4γ binding pocket in the first solvent-flattened SIRAS map. During the course of refinement, the pocket density improved and appeared consistent with a thin curved ligand, depicted in FIG. 1B. The ligand density starts adjacent to residue R186, curves around the M142-M301 bridge and proceeds towards HNF4γ residue V314.

The description of the bound ligand from the structural data led to the belief that the compound was a fatty acid. Analytical methods were used to obtain a definitive identification of the ligand. First, bound ligand(s) was separated from a purified preparation of HNF4γ LBD by liquid-liquid extraction (Folch et al., (1957) J. Biol. Chem. 226: 497-509). The extract was then treated with 3% (v/v) acetyl chloride in methanol. This reagent converts fatty acids to their corresponding fatty acid methyl esters (FAME). The derivatized sample was then analyzed by gas chromatography/mass spectrometry (GC/MS) using both electron impact ionization (EI) and chemical ionization (CI) in separate analyses. The constituents of the extract were identified by comparing the GC/MS data for the extract with data for standard fatty acids, acquired likewise.

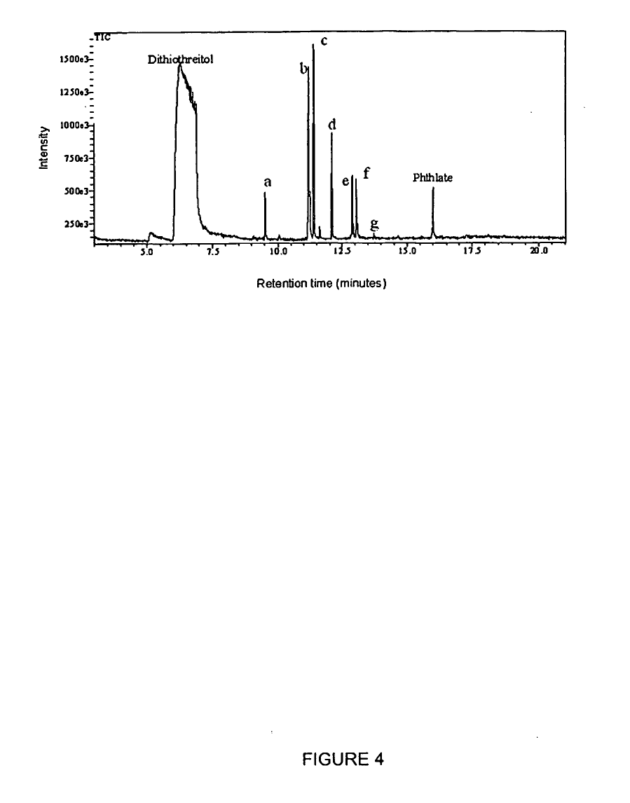

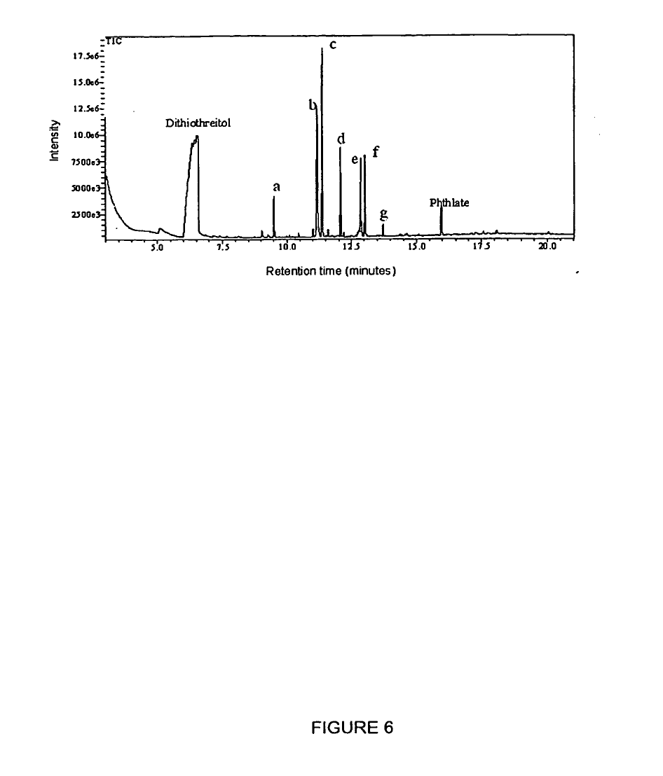

FIG. 4 shows the total ion current (TIC) chromatogram from the analysis of the derivatized extract by GCMS with CI. A similar TIC chromatogram was obtained from the EI analysis as shown in FIG. 6. The CI mass spectra for peaks a through g all show a protonated molecular ion ([M+H]⁺) along with a fragment ion at a mass-to-charge (m/z) value 32 Da below the protonated molecular ion. This fragmentation is common in CI mass spectra for FAME and represents the loss of methanol from the protonated methyl ester. The CI mass spectrum for peak c is shown in FIG. 6. It was identified as the methyl ester of palmitic acid.

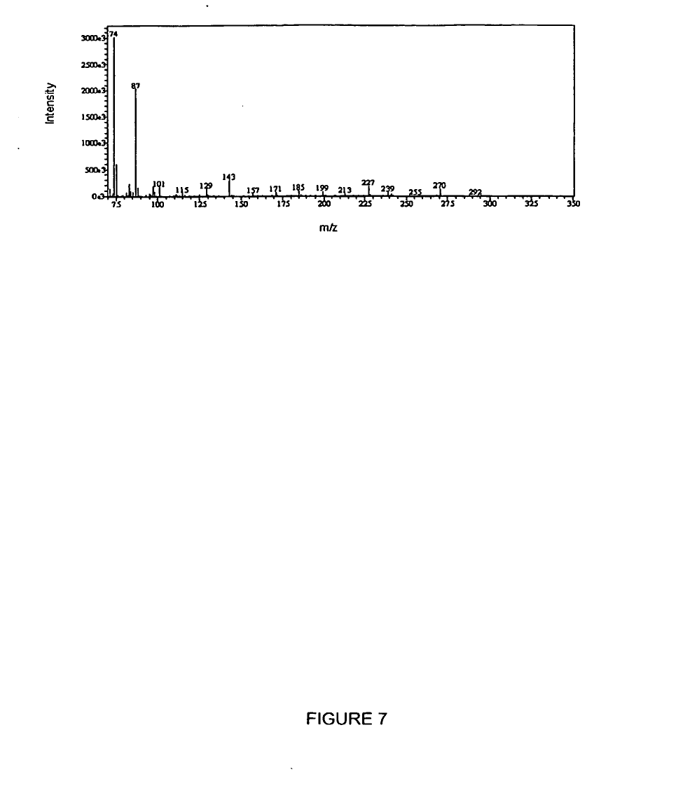

A comparison of FIG. 4 and FIG. 6, reveals that peaks a-g that were present in the TIC from the CI analysis were also present in the TIC from the EI analysis. The EI spectrum for peak c is shown in FIG. 7 and shows the molecular ion for methyl palmitate at m/z 270. The 14 Da mass difference observed for the fragment ions in this spectrum is characteristic of EI mass spectra of long chain aliphatic compounds such as fatty acids. A similar fragmentation pattern was observed in the EI spectrum for all of peaks a through g.

Results of the GC/MS analyses show that the HNF4γ extract consisted of a mixture of fatty acids with palmitic acid as the most abundant component. Data from the CI analysis are summarized in Table 4. The second column lists the protonated molecular ion detected of each labeled peak in FIG. 4. The third column gives the predicted molecular weight of the free acid form for each component in the extract.

VI.E. Confirmation of the Functionality of the HNF4 Ligand by FRET Assay

To confirm that fatty acids were functional HNF4 ligands, HNF4α and HNF4γ were tested for their ability to recruit the nuclear receptor co-activator CREB binding protein, a known activation partner (Wang et al., (1998) J. Biol. Chem. 273: 30847-50; Dell & Hadzopoulou-Cladaras, (1999) J. Biol. Chem. 274: 9013-21). A FRET (fluorescent resonance energy transfer) assay was employed using purified recombinant CREB-binding protein (CBP) and HNF4 LBD (Zhou et al., (1998) Mol. Endocrinol. 12: 1594-1604). Long-chain fatty acids (LCFA) with increasing carbon lengths from 12 to 18 carbon methylene units were tested for their ability to modulate the association between HNF4 and CBP in a dose dependent manner. Saturated fatty acids with chains smaller than 16 carbons did not affect basal CBP association. Palmitic and stearic acids increased the allosteric interaction between CBP and HNF4, with apparent binding constants of 1 μM.

The reported ligands for HNF4α are fatty acyl-CoA thioesters (Hertz et al., (1998) Nature (London) 392: 512-16), which are much larger than other nuclear receptor ligands (Bogan et al., (1998) Nat. Struct. Biol. 5: 679-81). When tested in the FRET assay, palmitoyl-CoA and steroyl-CoA decreased the basal level of CBP recruitment to both HNF4α and HNF4γ. Shorter fatty acyl-CoAs had no effect on CBP association. This behavior indicates that longer chain fatty-acyl CoA derivatives are not HNF4 agonists.

HNF4α is primarily expressed in the liver and pancreas and is regulated by fatty acids, indicating a link between fatty acid and glucose metabolism. There are known effects of free fatty acids on glucose-stimulated insulin secretion (GSIS), including an initial stimulatory effect (Stein et al., (1997) J. Clin. Invest 100:398403; Dobbins et al., (1998a) Diabetes 47:1613-18; Dobbins et al., (1998b) J. Clin. Invest. 101:2370-76), followed by a decrease after long term exposure (Zhou & Grill, (1994) J. Clin. Invest. 93:870-76; Zhou & Grill, (1995) J. Clin. Endocrinol. Metab. 80:1584-90; Boliheimer et al., (1998) J. Clin. Invest. 101:1094-1101; Biorklund & Grill (1999) Diabetes 48:1409-14; Jacqueminet et al., (2000) Metab. Clin. Exp. 49:532-36). The observed negative effects of long term fatty acid exposure on pancreatic islet function (Zhou & Grill, (1995) J. Clin. Endocrinol Metab. 80:1584-90) are likely to be partially mediated by HNF4.

VI.F. Analysis of the HNF4α Ligand Binding Mode

Although fatty acids are ligands for both PPARs and HNF4s, the proposed binding mode and specificity are significantly different. The structure of EPA bound to PPARδ (Xu et al., (1999) MOL Cell 3: 397-403) showed that the acid head group hydrogen bonds to PPARδ residues H323, H449 and Y43 in the AF2 helix. In HNF4γ, the fatty acid head group most likely hydrogen bonds to residue R185 in helix 5, and possibly to G197, much like the acid-protein interactions observed in retinoid binding nuclear receptors (Bourguet et al., (2000)Mol. Cell 5: 289-98; Gampe, Jr. et al., (2000) Mol. Cell 5: 545-55; Renaud et al., (1995) Nature 378: 681-89). The hydrophobic tail in the PPARδ/EPA complex can adopt two bent conformations, with the tail-up conformation pointing towards helix 5. In contrast, the hydrophobic tail in HNF4γ curves around the M142-M301 salt bridge and points towards the loop between helix 11 and the AF2 helix. Thus, the fatty acid in PPARδ binds in essentially the reverse orientation to that in HNF4γ.

The substrate specificity of the HNF4s is also markedly different from PPARs. The PPARs accept a wide range of fatty acids, but C18-20 mono- and poly-unsaturated fatty acids bind most tightly. Both HNF4s bind a much smaller range of substrates, with 16-18 carbon saturated fatty acids highly preferred. Thus, all HNF4 substrates are also bound by PPARα and PPARδ, but the converse is not true. The greater substrate specificity of HNF4 indicates a more specific role in the regulation of biological pathways.

VI.G Unique Structural Differences Between HNF4, and HNF4α

Without an atomic structure for HNF4α, the structure of HNF4γ can be considered in order to speculate on the design of isoform specific compounds. The solved structure of HNF4γ suggests that there is a potential for isoform specific ligand recognition based on amino acid differences between HNF4α and HNF4γ. Of the 26 amino acids in the binding pocket, 6 are different between HNF4γ and HNF4α. The substitution that can be directly exploited for designing isoform specific ligands is Ala215γ-Ser256α. This substitution adds a hydrogen bond donor near the C₈-C₉ of palmitic acid, and represents a substantial change to the chemical character of the binding pocket. Compounds that make this hydrogen bond will preferentially bind to HNF4α. Alternatively, compounds with a bulky hydrophobic group in that position may clash sterically with the hydroxyl of serine, and would preferentially bind HNF4γ. Thus, the HNF4γ structure provides a roadmap for the design of isoform specific compounds.

Most of the substitutions between HNF4α and HNF4γ are conservative, exchanging one hydrophobic residue for another. These are Ile202γ-Val242α, Ile211γ-Met252α, Val218γ-Ile259α, Val308γ-Ile349α, and Val314γ-Ala355α. These substitutions have the effect of changing the shape of the binding pocket without altering its chemical characteristics greatly. Two of the substitutions that add mass to the binding pocket residues (Ile211γ-Met252α, Val218γ-Ile259α) occur along the curve of palmitic acid, and have the effect of restricting the pocket. This is partially offset by the substitution Ile202γ-Val242α near palmitic acid C₆-C₈, which enlarges a cavity in the binding pocket. The pair of substitutions Val308γ-Ile349α, and Val314γ-Ala355α occur near the paimitic acid tail, and direct the fatty acid tail more towards the loop connecting helix 11 and helix 12 (the AF2 helix), while expanding the pocket there. These shape changes to the binding pocket can also be exploited in the design of isoform specific compounds.

One other difference between HNF4γ and HNF4α that could change the characteristics of the binding pocket is that HNF4α has an extra residue, Ala250, in the loop between the beta turn and helix 7. This extra residue could slightly shift the positions of the residues in helix 7, i.e. Glu251, Met252, Val255, Ser256, and Ile259. However, modeling amino acid shifts caused by extra loop residues is more speculative than substitutions.

VI.H. Generation of Easily-Solved HNF4 Crystals

The present invention discloses a substantially pure HNF4 LBD polypeptide in crystalline form. In a preferred embodiment, exemplified in the Figures and Laboratory Examples, HNF4γ is crystallized with bound ligand. Crystals are formed from HNF4 LBD polypeptides that are usually expressed by a cell culture, such as E. coli. Bromo-, iodo- and substitutions can be included during the preparation of crystal forms and can act as heavy atom substitutions in HNF4 ligands and crystals of HNF4s. This method can be advantageous for the phasing of the crystal, which is a crucial, and sometimes limiting, step in solving the three-dimensional structure of a crystallized entity. Thus, the need for generating the heavy metal derivatives traditionally employed in crystallography can be eliminated. After the three-dimensional structure of an HNF4 or HNF4 LBD with or without a ligand bound is determined, the resultant three-dimensional structure can be used in computational methods to design synthetic ligands for HNF4γ and other HNF4 polypeptides. Further activity structure relationships can be determined through routine testing, using assays disclosed herein and known in the art.

VII. Uses of HNF4γ Crystals and the Three-Dimensional Structure of the Ligand Bindina Domain of HNF4γ

VII.A. Design and Development of HNF4 Modulators

The knowledge of the structure of the HNF4γ ligand binding domain (LBD), an aspect of the present invention, provides a tool for investigating the mechanism of action of HNF4γ and other HNF4 polypeptides in a subject. For example, various computer models, as described herein, can predict the binding of various substrate molecules to the LBD of HNF4γ. Upon discovering that such binding in fact takes place, knowledge of the protein structure then allows design and synthesis of small molecules that mimic the functional binding of the substrate to the LBD of HNF4γ, and to the LBDs of other HNF4 polypeptides. This is the method of “rational” drug design, further described herein.

Use of the isolated and purified HNF4γ crystalline structure of the present invention in rational drug design is thus provided in accordance with the present invention. Additional rational drug design techniques are described in U.S. Pat. Nos. 5,834,228 and 5,872,011, incorporated herein in their entirety.

Thus, in addition to the compounds described herein, other sterically similar compounds can be formulated to mimic the key structural regions of an HNF4 in general, or of HNF4γ in particular. The generation of a structural functional equivalent can be achieved by the techniques of modeling and chemical design known to those of skill in the art and described herein. It will be understood that all such sterically similar constructs fall within the scope of the present invention.

VII.A.1. Rational Drug Design

The three-dimensional structure of the ligand binding domain of HNF4γ is unprecedented and will greatly aid in the development of new synthetic ligands for an HNF4 polypeptide, such as HNF4 agonists and antagonists, including those that bind exclusively to any one of the HNF4 isoforms. In addition, the HNF4s are well suited to modern methods, including three-dimensional structure elucidation and combinatorial chemistry, such as those disclosed in U.S. Pat. No. 5,463,564, incorporated herein by reference. Structure determination using X-ray crystallography is possible because of the solubility properties of the HNF4s. Computer programs that use crystallography data when practicing the present invention will enable the rational design of ligands to these receptors. Programs such as RASMOL (Biomolecular Structures Group, Glaxo Wellcome Research & Development Stevenage, Hertfordshire, UK Version 2.6, August 1995, Version 2.6.4, December 1998, Copyright © Roger Sayle 1992-1999) can be used with the atomic structural coordinates from crystals generate by practicing the invention or used to practice the invention by generating three-dimensional models and/or determining the structures involved in ligand binding. Computer programs such as those sold under the registered trademark INSIGHT II® and such as GRASP (Nicholls et al., (1991) Proteins 11: 282) allow for further manipulations and the ability to introduce new structures. In addition, high throughput binding and bioactivity assays can be devised using purified recombinant protein and modern reporter gene transcription assays known to those of skill in the art in order to refine the activity of a designed ligand.

A method of identifying modulators of the activity of an HNF4 polypeptide using rational drug design is thus provided in accordance with the present invention. The method comprises designing a potential modulator for an HNF4 polypeptide of the present invention that will form non-covalent bonds with amino acids in the ligand binding pocket based upon the crystalline structure of the HNF4γ LBD polypeptide; synthesizing the modulator; and determining whether the potential modulator modulates the activity of the HNF4 polypeptide. In a preferred embodiment, the modulator is designed for an HNF4γ polypeptide. Preferably, the HNF4γ polypeptide comprises the nucleic acid sequence of SEQ ID NO:1, and the HNF4γ LBD comprises the nucleic acid sequence SEQ ID NO:3. The determination of whether the modulator modulates the biological activity of an HNF4 polypeptide is made in accordance with the screening methods disclosed herein, or by other screening methods known to those of skill in the art. Modulators can be synthesized using techniques known to those of ordinary skill in the art.

In an alternative embodiment, a method of designing a modulator of an HNF4 polypeptide in accordance with the present invention is disclosed comprising: (a) selecting a candidate HNF4 ligand; (b) determining which amino acid or amino acids of an HNF4 polypeptide interact with the ligand using a three-dimensional model of a crystallized HNF4γ LBD; (c) identifying in a biological assay for HNF4 activity a degree to which the ligand modulates the activity of the HNF4 polypeptide; (d) selecting a chemical modification of the ligand wherein the interaction between the amino acids of the HNF4 polypeptide and the ligand is predicted to be modulated by the chemical modification; (e) performing the chemical modification on the ligand to form a modified ligand; (f) contacting the modified ligand with the HNF4 polypeptide; (g) identifying in a biological assay for HNF4 activity a degree to which the modified ligand modulates the biological activity of the HNF4 polypeptide; and (h) comparing the biological activity of the HNF4 polypeptide in the presence of modified ligand with the biological activity of the HNF4 polypeptide in the presence of the unmodified ligand, whereby a modulator of an HNF4 polypeptide is designed. present invention. The method comprises designing a potential modulator for an HNF4 polypeptide of the present invention that will form non-covalent bonds with amino acids in the ligand binding pocket based upon the crystalline structure of the HNF4γ LBD polypeptide; synthesizing the modulator; and determining whether the potential modulator modulates the activity of the HNF4 polypeptide. In a preferred embodiment, the modulator is designed for an HNF4γ polypeptide. Preferably, the HNF4γ polypeptide comprises the nucleic acid sequence of SEQ ID NO:1, and the HNF4γ LBD comprises the nucleic acid sequence SEQ ID NO:3. The determination of whether the modulator modulates the biological activity of an HNF4 polypeptide is made in accordance with the screening methods disclosed herein, or by other screening methods known to those of skill in the art. Modulators can be synthesized using techniques known to those of ordinary skill in the art.

In an alternative embodiment, a method of designing a modulator of an HNF4 polypeptide in accordance with the present invention is disclosed comprising: (a) selecting a candidate HNF4 ligand; (b) determining which amino acid or amino acids of an HNF4 polypeptide interact with the ligand using a three-dimensional model of a crystallized HNF4γ LBD; (c) identifying in a biological assay for HNF4 activity a degree to which the ligand modulates the activity of the HNF4 polypeptide; (d) selecting a chemical modification of the ligand wherein the interaction between the amino acids of the HNF4 polypeptide and the ligand is predicted to be modulated by the chemical modification; (e) performing the chemical modification on the ligand to form a modified ligand; (f) contacting the modified ligand with the HNF4 polypeptide; (g) identifying in a biological assay for HNF4 activity a degree to which the modified ligand modulates the biological activity of the HNF4 polypeptide; and (h) comparing the biological activity of the HNF4 polypeptide in the presence of modified ligand with the biological activity of the HNF4 polypeptide in the presence of the unmodified ligand, whereby a modulator of an HNF4 polypeptide is designed.

VII.A.2. Methods for Using the HNF4γ LBD Structural Coordinates for Molecular Design

For the first time, the present invention permits the use of molecular design techniques to design, select and synthesize chemical entities and compounds, including modulatory compounds, capable of binding to the ligand binding pocket or an accessory binding site of HNF4γ and the HNF4γ LBD, in whole or in part. Correspondingly, the present invention also provides for the application of similar techniques in the design of modulators of any HNF4 polypeptide.

In accordance with a preferred embodiment of the present invention, the structure coordinates of a crystalline HNF4γ LBD can be used to design compounds that bind to an HNF4 LBD (more preferably an HNF4γ LBD) and alter the properties of an HNF4 LBD (for example, the dimerization or ligand binding ability) in different ways. One aspect of the present invention provides for the design of compounds that act as competitive inhibitors of an HNF4 polypeptide by binding to all, or a portion of, the binding sites on an HNF4 LBD. The present invention also provides for the design of compounds that can act as uncompetitive inhibitors of an HNF4 LBD. These compounds can bind to all, or a portion of, an accessory binding site of an HNF4 that is already binding its ligand and can, therefore, be more potent and less non-specific than known competitive inhibitors that compete only for the HNF4 ligand binding pocket. Similarly, non-competitive inhibitors that bind to and inhibit HNF4 LBD activity, whether or not it is bound to another chemical entity, can be designed using the HNF4 LBD structure coordinates of this invention.

A second design approach is to probe an HNF4 or HNF4 LBD (preferably an HNF4γ or HNFγ LBD) crystal with molecules comprising a variety of different chemical entities to determine optimal sites for interaction between candidate HNF4 or HNF4 LBD modulators and the polypeptide. For example, high resolution X-ray diffraction data collected from crystals saturated with solvent allows the determination of the site where each type of solvent molecule adheres. Small molecules that bind tightly to those sites can then be designed and synthesized and tested for their HNF4γ modulator activity.

Once a computationally-designed ligand is synthesized using the methods of the present invention or other methods known to those of skill in the art, assays can be used to establish its efficacy of the ligand as a modulator of HNF4 (preferably HNF4γ) activity. After such assays, the ligands can be further refined by generating intact HNF4, or HNF4 LBD, crystals with a ligand bound to the LBD. The structure of the ligand can then be further refined using the chemical modification methods described herein and known to those of skill in the art, in order to improve the modulation activity or the binding affinity of the ligand. This process can lead to second generation ligands with improved properties.

Ligands also can be selected that modulate HNF4 responsive gene transcription by the method of altering the interaction of co-activators and co-repressors with their cognate HNF4. For example, agonistic ligands can be selected that block or dissociate a co-repressor from interacting with the HNF4, and/or that promote binding or association of a co-activator. Antagonistic ligands can be selected that block co-activator interaction and/or promote co-repressor interaction with a target receptor. Selection can be done via binding assays that screen for designed ligands having the desired modulatory properties. Preferably, interactions of an HNF4γ polypeptide are targeted. Suitable assays for screening that can be employed, mutatis mutandis in the present invention, are described in published PCT international applications WO 00/037,077 and WO 00/025,134, which are incorporated herein in their entirety by reference.

VII.A.3. Methods of Designing HNF4 LBD Modulator Compounds

The design of candidate substances, also referred to as “compounds” or “candidate compounds”, that bind to or inhibit HNF4 LBD-mediated activity according to the present invention generally involves consideration of two factors. First, the compound must be capable of physically and structurally associating with an HNF4 LBD. Non-covalent molecular interactions important in the association of an HNF4 LBD with its substrate include hydrogen bonding, van der Waals interactions and hydrophobic interactions.

Second, the compound must be able to assume a conformation that allows it to associate with an HNF4 LBD. Although certain portions of the compound will not directly participate in this association with an HNF4 LBD, those portions can still influence the overall conformation of the molecule. This, in turn, can have a significant impact on potency. Such conformational requirements include the overall three-dimensional structure and orientation of the chemical entity or compound in relation to all or a portion of the binding site, e.g., the ligand binding pocket or an accessory binding site of an HNF4 LBD, or the spacing between functional groups of a compound comprising several chemical entities that directly interact with an HNF4 LBD.

The potential modulatory or binding effect of a chemical compound on an HNF4 LBD can be analyzed prior to its actual synthesis and testing by the use of computer modeling techniques that employ the coordinates of a crystalline HNF4γ LBD polypeptide of the present invention. If the theoretical structure of the given compound suggests insufficient interaction and association between it and an HNF4 LBD, synthesis and testing of the compound is obviated. However, if computer modeling indicates a strong interaction, the molecule can then be synthesized and tested for its ability to bind and modulate the activity of an HNF4 LBD. In this manner, synthesis of unproductive or inoperative compounds can be avoided.

A modulatory or other binding compound of an HNF4 LBD polypeptide (preferably an HNF4γ LBD) can be computationally evaluated and designed via a series of steps in which chemical entities or fragments are screened and selected for their ability to associate with the individual binding sites or other areas of a crystalline HNF4γ LBD polypeptide of the present invention.

One of several methods can be used to screen chemical entities or fragments for their ability to associate with an HNF4 LBD and, more particularly, with the individual binding sites of an HNF4 LBD, such as ligand binding pocket or an accessory binding site. This process can begin by visual inspection of, for example, the ligand binding pocket on a computer screen based on the HNF4γ LBD atomic coordinates in Table 2. Selected fragments or chemical entities can then be positioned in a variety of orientations, or docked, within an individual binding site of an HNF4γ LBD as defined herein above. Docking can be accomplished using software programs such as those available under the tradenames QUANTA™ (Molecular Simulations Inc., San Diego, Calif.) and SYBYL™ (Tripos, Inc., St. Louis, Mo.), followed by energy minimization and molecular dynamics with standard molecular mechanics forcefields, such as CHARM (Brooks et al., (1983) J. Comp. Chem., 8: 132) and AMBER 5 (Case et al., (1997), AMBER 5, University of California, San Francisco; Pearlman et al., (1995) Comput. Phys. Commun. 91: 1-41).

Specialized computer programs can also assist in the process of selecting fragments or chemical entities. These include:
1. GRID™ program, version 17 (Goodford, (1985) J. Med. Chem. 28: 849-57), which is available from Molecular Discovery Ltd., Oxford, UK;
2. MCSS™ program (Miranker & Karplus, (1991) Proteins 11: 29-34), which is available from Molecular Simulations, Inc., San Diego, Calif.;
3. AUTODOCK™ 3.0 program (Goodsell & Olsen, (1990) Proteins 8: 195-202), which is available from the Scripps Research Institute, La Jolla, Calif.;
4. DOCK™ 4.0 program (Kuntz et al., (1992) J. Mol. Biol. 161: 269-88), which is available from the University of California, San Francisco, Calif.;
5. FLEX-X™ program (See, Rarey et al., (1996) J. Comput Aid. Mol Des. 10:41-54), which is available from Tripos, Inc., St. Louis, Mo.;
6. MVP program (Lambert, (1997) in Practical Application of Computer-Aided Drug Design, (Charifson, ed.) Marcel-Dekker, New York, pp. 243-303); and
7. LUDI™ program (Bohm, (1992) J. Comput Aid. Mol. Des., 6: 61-78), which is available from Molecular Simulations, Inc., San Diego, Calif.

Once suitable chemical entities or fragments have been selected, they can be assembled into a single compound or modulator. Assembly can proceed by visual inspection of the relationship of the fragments to each other on the three-dimensional image displayed on a computer screen in relation to the structure coordinates of an HNF4γ LBD. Manual model building using software such as QUANTA™ or SYBYL™ typically follows.

Useful programs to aid one of ordinary skill in the art in connecting the individual chemical entities or fragments include:
1. CAVEAT™ program (Bartlett et al., (1989) Special Pub., Royal Chem. Soc. 78: 182-96), which is available from the University of California, Berkeley, Calif.;
2. 3D Database systems, such as MACCS-3D™ system program, which is available from MDL Information Systems, San Leandro, Calif.. This area is reviewed in Martin, (1992) J. Med. Chem. 35: 2145-54; and
3. HOOK™ program (Eisen et al., (1994). Proteins 19: 199-221), which is available from Molecular Simulations, Inc., San Diego, Calif.

Instead of proceeding to build an HNF4 LBD modulator (preferably an HNF4γ LBD modulator) in a step-wise fashion one fragment or chemical entity at a time as described above, modulatory or other binding compounds can be designed as a whole or de novo using the structural coordinates of a crystalline HNF4γ LBD polypeptide of the present invention and either an empty binding site or optionally including some portion(s) of a known modulator(s). Applicable methods can employ the following software programs:
1. LUDI™ program (Bohm, (1992) J. Comput. Aid. Mol. Des., 6: 61-78), which is available from Molecular Simulations, Inc., San Diego, Calif.;
2. LEGEND™ program (Nishibata & ltai, (1991) Tetrahedron 47: 8985); and
3. LEAPFROG™, which is available from Tripos Associates, St. Louis, Mo.

Other molecular modeling techniques can also be employed in accordance with this invention. See, e.g., Cohen et al., (1990) J. Med. Chem. 33: 883-94. See also, Navia & Murcko, (1992) Curr. Opin. Struc. Biol. 2: 202-10; U.S. Pat. No. 6,008,033, herein incorporated by reference.

Once a compound has been designed or selected by the above methods, the efficiency with which that compound can bind to an HNF4γ LBD can be tested and optimized by computational evaluation. By way of particular example, a compound that has been designed or selected to function as an HNF4γ LBD modulator should also preferably traverse a volume not overlapping that occupied by the binding site when it is bound to its native ligand. Additionally, an effective HNF4 LBD modulator should preferably demonstrate a relatively small difference in energy between its bound and free states (i.e., a small deformation energy of binding). Thus, the most efficient HNF4 LBD modulators should preferably be designed with a deformation energy of binding of not greater than about 10 kcal/mole, and preferably, not greater than 7 kcal/mole. It is possible for HNF4 LBD modulators to interact with the polypeptide in more than one conformation that is similar in overall binding energy. In those cases, the deformation energy of binding is taken to be the difference between the energy of the free compound and the average energy of the conformations observed when the modulator binds to the polypeptide.

A compound designed or selected as binding to an HNF4 polypeptide (preferably an HNF4γ LBD polypeptide) can be further computationally optimized so that in its bound state it would preferably lack repulsive electrostatic interaction with the target polypeptide. Such non-complementary (e.g., electrostatic) interactions include repulsive charge-charge, dipole-dipole and charge-dipole interactions. Specifically, the sum of all electrostatic interactions between the modulator and the polypeptide when the modulator is bound to an HNF4 LBD preferably make a neutral or favorable contribution to the enthalpy of binding.

Specific computer software is available in the art to evaluate compound deformation energy and electrostatic interaction. Examples of programs designed for such uses include:
1. Gaussian 98™, which is available from Gaussian, Inc., Pittsburgh, Pa.;
2. AMBER™ program, version 6.0, which is available from the University of California at San Francisco;
3. QUANTA™ program, which is available from Molecular Simulations, Inc., San Diego, Calif.;
4. CHARMm® program, which is available from Molecular Simulations, Inc., San Diego, Calif.; and
4. Insight II® program, which is available from Molecular Simulations, Inc., San Diego, Calif.

These programs can be implemented using a suitable computer system. Other hardware systems and software packages will be apparent to those skilled in the art after review of the disclosure of the present invention presented herein.

Once an HNF4 LBD modulating compound has been optimally selected or designed, as described above, substitutions can then be made in some of its atoms or side groups in order to improve or modify its binding properties. Generally, initial substitutions are conservative, i.e., the replacement group will have approximately the same size, shape, hydrophobicity and charge as the original group. It should, of course, be understood that components known in the art to alter conformation should be avoided. Such substituted chemical compounds can then be analyzed for efficiency of fit to an HNF4 LBD binding site using the same computer-based approaches described in detail above.

VII.B. Distinguishing Between HNF4 Isoforms

The present invention discloses the ability to generate new synthetic ligands to distinguish between HNF4 isoforms. As described herein, computer-designed ligands can be generated that distinguish between binding isoforms, thereby allowing the generation of either tissue specific or function specific ligands. The atomic structural coordinates disclosed in the present invention reveal structural details unique to HNF4γ. These structural details can be exploited when a novel ligand is designed using the methods of the present invention or other ligand design methods known in the art. The structural features that differentiate an HNF4γ from an HNF4α can be targeted in ligand design. Thus, for example, a ligand can be designed that will recognize HNF4γ, while not interacting with other HNF4s or even with moieties having similar structural features. Prior to the disclosure of the present invention, the ability to target an HNF4 isoform was unattainable.

VII.C. Method of Screening for Chemical and Biological Modulators of the Biological Activity of HNF4γ

A candidate substance identified according to a screening assay of the present invention has an ability to modulate the biological activity of an HNF4 polypeptide or an HNF4 LBD polypeptide. In a preferred embodiment, such a candidate compound can have utility in the treatment of disorders and conditions associated with the biological activity of an HNF4γ or an HNF4γ LBD polypeptide, including diabetes, glucose homeostasis and lipid homeostasis.

In a cell-free system, the method comprises the steps of establishing a control system comprising an HNF4γ polypeptide and a ligand which is capable of binding to the polypeptide; establishing a test system comprising an HNF4γ polypeptide, the ligand, and a candidate compound; and determining whether the candidate compound modulates the activity of the polypeptide by comparison of the test and control systems. A representative ligand comprises a fatty acid or other small molecule, and in this embodiment, the biological activity or property screened includes binding affinity.

In another embodiment of the invention, a form of an HNF4γ polypeptide or a catalytic or immunogenic fragment or oligopeptide thereof, can be used for screening libraries of compounds in any of a variety of drug screening techniques. The fragment employed in such a screening can be affixed to a solid support. The formation of binding complexes, between an HNF4γ polypeptide and the agent being tested, will be detected. In a preferred embodiment, the HNF4γ polypeptide has an amino acid sequence of SEQ ID NO:2. When an HNF4γ LBD polypeptide is employed, a preferred embodiment will include an HNF4γ polypeptide having the amino acid sequence of SEQ ID NO:4.

Another technique for drug screening which can be used provides for high throughput screening of compounds having suitable binding affinity to the protein of interest as described in published PCT application WO 84/03564, herein incorporated by reference. In this method, as applied to a polypeptide of the present invention, large numbers of different small test compounds are synthesized on a solid substrate, such as plastic pins or some other surface. The test compounds are reacted with the polypeptide, or fragments thereof. Bound polypeptide is then detected by methods well known to those of skill in the art. The polypeptide can also be placed directly onto plates for use in the aforementioned drug screening techniques.

In yet another embodiment, a method of screening for a modulator of an HNF4γ polypeptide or an HNF4γ LBD polypeptide comprises: providing a library of test samples; contacting an HNF4γ polypeptide or an HNF4γ LBD polypeptide with each test sample; detecting an interaction between a test sample and a an HNF4γ polypeptide or an HNF4γ LBD polypeptide; identifying a test sample that interacts with an HNF4γ polypeptide or an HNF4γ LBD polypeptide; and isolating a test sample that interacts with an HNF4γ polypeptide or an HNF4γ LBD polypeptide.

In each of the foregoing embodiments, an interaction can be detected spectrophotometrically, radiologically or immunologically. An interaction between an HNF4γ polypeptide or an HNF4γ LBD polypeptide and a test sample can also be quantified using methodology known to those of skill in the art. In another embodiment, the HNF4γ polypeptide and the HNF4γ LBD is in crystalline form.

In accordance with the present invention there is also provided a rapid and high throughput screening method that relies on the methods described above. This screening method comprises separately contacting each of a plurality of substantially identical samples with an HNF4γ polypeptide or an HNF4γ LBD and detecting a resulting binding complex. In such a screening method the plurality of samples preferably comprises more than about 10⁴ samples, or more preferably comprises more than about 5×10⁴ samples.

VII.D. Method of Identifying Compounds Which Inhibit Ligand Binding

Until disclosure of the present invention, the natural ligand of HNF4γ was unknown. Various hypotheses predicted the general properties an HNF4γ ligand might exhibit, but no ligand was conclusively identified. The present invention solves this problem by conclusively identifying a natural ligand of HNF4γ, the fatty acid palmitic acid. Using the identity of HNF4γ's natural ligand, disclosed for the first time herein, it is possible to design test compounds that inhibit binding of ligands normally bound by an HNF4 polypeptide.

In one aspect of the present invention, an assay method for identifying a compound that inhibits binding of a ligand to an HNF4 polypeptide is disclosed. A natural ligand of HNF4γ, such as a fatty acid can be used in the assay method as the ligand against which the inhibition by a test compound is gauged. Palmitic acid is a preferred fatty acid in the assay method. The method comprises (a) incubating an HNF4 polypeptide with a ligand in the presence of a test inhibitor compound; (b) determining an amount of ligand that is bound to the HNF4 polypeptide, wherein decreased binding of ligand to the HNF4 polypeptide in the presence of the test inhibitor compound relative to binding in the absence of the test inhibitor compound is indicative of inhibition; and (c) identifying the test compound as an inhibitor of ligand binding if decreased ligand binding is observed. Preferably, the ligand is a fatty acid and even more preferably, the fatty acid is palmitic acid.

In another aspect of the present invention, the disclosed assay method can be used in the structural refinement of candidate HNF4 inhibitors. For example, multiple rounds of optimization can be followed by gradual structural changes in a strategy of inhibitor design. A strategy such as this is made possible by the disclosure of the coordinates of the HNF4γ LBD and the disclosure of a natural ligand of HNF4, the fatty acid, palmitic acid.

VII.E. Design of HNF4 Isoform Modulators

The HNF4γ crystal structure of the present invention can be used to generate modulators of other HNF4 isoforms, such as HNF4α. Analysis of the disclosed crystal structure can provide a guide for designing HNF4α modulators. Absent the crystal structure of the present invention, researches would be required to design HNF4α modulators de novo. The present invention, however, addresses this problem by providing insights into the binding pocket of HNF4γ which can be extended, due to significant structural similarity, to the binding pocket of HNF4α. An evaluation of the binding pocket of HNF4γ indicates that a potential HNF4α modulator would meet a broad set of general criteria. Broadly, it can be stated that, based on the crystal structure of HNF4γ, a potent HNF4α ligand would require several general features including: (a) a carboxylic acid or equivalent isosteric “head group” to interact with the amino acids R186 and G197 to form a strong polar hydrogen bonding interaction; (b) a lipophilic non-head group region of the molecule, which could possibly consist of aromatic rings, aliphatic carbon atoms, ether oxygens atoms, etc.; and (c) the ability to adopt a conformation that is complementary to the shape of the binding pocket.

Using the discerned structural similarities and differences between HNF4 isoforms, as represented and predicted based on the crystal structure of the present invention and homology models, an HNF4α modulator can be designed. For example, based on an evaluation of a homology model of HNF4α, which is derived from the HNF4γ crystal structure, it is expected that a potent ligand would need similar characteristics as listed above for a compound recognized by HNF4γ. Additional modifications can be included, based on the disclosed structure, which are predicted to further define a modulator specific for HNF4α over other isoforms. For example, if amino acid A215 (using HNF4γ numbering scheme) is mutated to a serine residue, a group capable of hydrogen bonding (which could be either donating or accepting) placed within 3 angstroms of the serine residue (distance of OG of the serine residue to the “heavy atom” of the hydrogen bonding group) would increase both the potency and selectivity of the compounds for HNF4α. Thus, the disclosed crystal structure of HNF4γ can be useful when designing modulators of HNF4α and other isoforms.

VII. Design. Preparation and Structural Analysis of HNF4γ and HNF4γ LBD Mutants and Structural Equivalents

The present invention provides for the generation of HNF4 and HNF4 mutants (preferably HNF4γ and HNF4γ LBD mutants), and the ability to solve the crystal structures of those that crystallize. More particularly, through the provision of the three-dimensional structure of an HNF4γ LBD, desirable sites for mutation can be identified.

The structure coordinates of an HNF4γ LBD provided in accordance with the present invention also facilitate the identification of related proteins or enzymes analogous to HNF4γ in function, structure or both, (for example, an HNF4α), which can lead to novel therapeutic modes for treating or preventing a range of disease states.

VIII.A. Sterically Similar Compounds

A further aspect of the present invention is that sterically similar compounds can be formulated to mimic the key portions of an HNF4 LBD structure. Such compounds are functional equivalents. The generation of a structural functional equivalent can be achieved by the techniques of modeling and chemical design known to those of skill in the art and described herein. Modeling and chemical design of HNF4 and HNF4 LBD structural equivalents can be based on the structure coordinates of a crystalline HNF4γ LBD polypeptide of the present invention. It will be understood that all such sterically similar constructs fall within the scope of the present invention.

VIII.B. HNF4 Polypeptides

The generation of chimeric HNF4 polypeptides is also an aspect of the present invention. Such a chimeric polypeptide can comprise an HNF4 LBD polypeptide or a portion of an HNF4 LBD, (e.g. an HNF4γ LBD) that is fused to a candidate polypeptide or a suitable region of the candidate polypeptide, for example HNF4α. Throughout the present disclosure it is intended that the term “mutant” encompass not only mutants of an HNF4 LBD polypeptide but chimeric proteins generated using an HNF4 LBD as well. It is thus intended that the following discussion of mutant HNF4 LBDs apply mutatis mutandis to chimeric HNF4 and HNF4 LBD polypeptides and to structural equivalents thereof.

In accordance with the present invention, a mutation can be directed to a particular site or combination of sites of a wild-type HNF4 LBD. For example, an accessory binding site or the binding pocket can be chosen for mutagenesis. Similarly, a residue having a location on, at or near the surface of the polypeptide can be replaced, resulting in an altered surface charge of one or more charge units, as compared to the wild-type HNF4 and HNF4 LBD. Alternatively, an amino acid residue in an HNF4 or an HNF4 LBD can be chosen for replacement based on its hydrophilic or hydrophobic characteristics.

Such mutants can be characterized by any one of several different properties as compared with the wild-type HNF4 LBD. For example, such mutants can have an altered surface charge of one or more charge units, or can have an increase in overall stability. Other mutants can have altered substrate specificity in comparison with, or a higher specific activity than, a wild-type HNF4 or HNF4 LBD.

HNF4 and HNF4 LBD mutants of the present invention can be generated in a number of ways. For example, the wild-type sequence of an HNF4 or an HNF4 LBD can be mutated at those sites identified using this invention as desirable for mutation, by means of oligonucleotide-directed mutagenesis or other conventional methods, such as deletion. Alternatively, mutants of an HNF4 or an HNF4 LBD can be generated by the site-specific replacement of a particular amino acid with an unnaturally occurring amino acid. In addition, HNF4 or HNF4 LBD mutants can be generated through replacement of an amino acid residue, for example, a particular cysteine or methionine residue, with selenocysteine or selenomethionine. This can be achieved by growing a host organism capable of expressing either the wild-type or mutant polypeptide on a growth medium depleted of either natural cysteine or methionine (or both) but enriched in selenocysteine or selenomethionine (or both).

Mutations can be introduced into a DNA sequence coding for an HNF4 or an HNF4 LBD using synthetic oligonucleotides. These oligonucleotides contain nucleotide sequences flanking the desired mutation sites. Mutations can be generated in the full-length DNA sequence of an HNF4 or an HNF4 LBD or in any sequence coding for polypeptide fragments of an HNF4 or an HNF4 LBD.

According to the present invention, a mutated HNF4 or HNF4 LBD DNA sequence produced by the methods described above, or any alternative methods known in the art, can be expressed using an expression vector. An expression vector, as is well known to those of skill in the art, typically includes elements that permit autonomous replication in a host cell independent of the host genome, and one or more phenotypic markers for selection purposes. Either prior to or after insertion of the DNA sequences surrounding the desired HNF4 or HNF4 LBD mutant coding sequence, an expression vector also will include control sequences encoding a promoter, operator, ribosome binding site, translation initiation signal, and, optionally, a repressor gene or various activator genes and a signal for termination. In some embodiments, where secretion of the produced mutant is desired, nucleotides encoding a “signal sequence” can be inserted prior to an HNF4 or an HNF4 LBD mutant coding sequence. For expression under the direction of the control sequences, a desired DNA sequence must be operatively linked to the control sequences; that is, the sequence must have an appropriate start signal in front of the DNA sequence encoding the HNF4 or HNF4 LBD mutant, and the correct reading frame to permit expression of that sequence under the control of the control sequences and production of the desired product encoded by that HNF4 or HNF4 LBD sequence must be maintained.

Any of a wide variety of well-known available expression vectors can be useful to express a mutated HNF4 or HNF4 LBD coding sequences of this invention. These include for example, vectors consisting of segments of chromosomal, non-chromosomal and synthetic DNA sequences, such as various known derivatives of SV40, known bacterial plasmids, e.g., plasmids from E. coli including col E1, pCR1, pBR322, pMB9 and their derivatives, wider host range plasmids, e.g., RP4, phage DNAs, e.g., the numerous derivatives of phage X, e.g., NM 989, and other DNA phages, e.g., M13 and filamentous single stranded DNA phages, yeast plasmids and vectors derived from combinations of plasmids and phage DNAs, such as plasmids which have been modified to employ phage DNA or other expression control sequences. In a preferred embodiment of this invention, the E. coli vector pRSET A, including a T7-based expression system, is employed.

In addition, any of a wide variety of expression control sequences-sequences that control the expression of a DNA sequence when operatively linked to it—can be used in these vectors to express the mutated DNA sequences according to this invention. Such useful expression control sequences, include, for example, the early and late promoters of SV40 for animal cells, the lac system, the trp system the TAC or TRC system, the major operator and promoter regions of phage X, the control regions of fd coat protein, all for E. coli, the promoter for 3-phosphoglycerate kinase or other glycolytic enzymes, the promoters of acid phosphatase, e.g., Pho5, the promoters of the yeast α-mating factors for yeast, and other sequences known to control the expression of genes of prokaryotic or eukaryotic cells or their viruses, and various combinations thereof.

A wide variety of hosts are also useful for producing mutated HNF4γ and HNF4γ LBD polypeptides according to this invention. These hosts include, for example, bacteria, such as E. coli, Bacillus and Streptomyces, fungi, such as yeasts, and animal cells, such as CHO and COS-1 cells, plant cells, insect cells, such as Sfg cells, and transgenic host cells.

It should be understood that not all expression vectors and expression systems function in the same way to express mutated DNA sequences of this invention, and to produce modified HNF4 and HNF4 LBD polypeptides or HNF4 or HNF4 LBD mutants. Neither do all hosts function equally well with the same expression system. One of skill in the art can, however, make a selection among these vectors, expression control sequences and hosts without undue experimentation and without departing from the scope of this invention. For example, an important consideration in selecting a vector will be the ability of the vector to replicate in a given host. The copy number of the vector, the ability to control that copy number, and the expression of any other proteins encoded by the vector, such as antibiotic markers, should also be considered.

In selecting an expression control sequence, a variety of factors should also be considered. These include, for example, the relative strength of the system, its controllability and its compatibility with the DNA sequence encoding a modified HNF4 or HNF4 LBD polypeptide of this invention, with particular regard to the formation of potential secondary and tertiary structures.

Hosts should be selected by consideration of their compatibility with the chosen vector, the toxicity of a modified HNF4 or HNF4 LBD to them, their ability to express mature products, their ability to fold proteins correctly, their fermentation requirements, the ease of purification of a modified HNF4 or HNF4 LBD and safety. Within these parameters, one of skill in the art can select various vector/expression control system/host combinations that will produce useful amounts of a mutant HNF4 or HNF4 LBD. A mutant HNF4 or HNF4 LBD produced in these systems can be purified by a variety of conventional steps and strategies, including those used to purify the wild-type HNF4 or HNF4 LBD.

Once an HNF4 LBD mutation(s) has been generated in the desired location, such as an active site or dimerization site, the mutants can be tested for any one of several properties of interest. For example, mutants can be screened for an altered charge at physiological pH. This is determined by measuring the mutant HNF4 or HNF4 LBD isoelectric point (pI) and comparing the observed value with that of the wild-type parent. Isoelectric point can be measured by gel-electrophoresis according to the method of Wellner (Wellner, (1971) Anal. Chem. 43: 597). A mutant HNF4 or HNF4 LBD polypeptide containing a replacement amino acid located at the surface of the enzyme, as provided by the structural information of this invention, can lead to an altered surface charge and an altered pl.

VIII.C. Generation of an Engineered HNF4 or HNF4 LBD Mutant

In another aspect of the present invention, a unique HNF4 or HNF4 LBD polypeptide can be generated. Such a mutant can facilitate purification and the study of the ligand-binding abilities of an HNF4 polypeptide.

As used in the following discussion, the terms “engineered HNF4”, “engineered HNF4 LDB”, “HNF4 mutant”, and “HNF4 LBD mutant” refers to polypeptides having amino acid sequences which contain at least one mutation in the wild-type sequence. The terms also refer to HNF4 and HNF4 LBD polypeptides which are capable of exerting a biological effect in that they comprise all or a part of the amino acid sequence of an engineered HNF4 or HNF4 LBD mutant polypeptide of the present invention, or cross-react with antibodies raised against an engineered HNF4 or HNF4 LBD mutant polypeptide, or retain all or some or an enhanced degree of the biological activity of the engineered HNF4 or HNF4 LBD mutant amino acid sequence or protein. Such biological activity can include lipid binding in general, and fatty acid binding in particular.

The terms “engineered HNF4 LBD” and “HNF4 LBD mutant” also includes analogs of an engineered HNF4 LBD or HNF4 LBD mutant polypeptide. By “analog” is intended that a DNA or polypeptide sequence can contain alterations relative to the sequences disclosed herein, yet retain all or some or an enhanced degree of the biological activity of those sequences. Analogs can be derived from genomic nucleotide sequences or from other organisms, or can be created synthetically. Those of skill in the art will appreciate that other analogs, as yet undisclosed or undiscovered, can be used to design and/or construct HNF4 LBD or HNF4 LBD mutant analogs. There is no need for an engineered HNF4 LBD or HNF4 LBD mutant polypeptide to comprise all or substantially all of the amino acid sequence of SEQ ID NOs:2 or 4. Shorter or longer sequences are anticipated to be of use in the invention; shorter sequences are herein referred to as “segments”. Thus, the terms “engineered HNF4 LBD” and “HNF4 LBD mutant” also includes fusion, chimeric or recombinant engineered HNF4 LBD or HNF4 LBD mutant polypeptides and proteins comprising sequences of the present invention. Methods of preparing such proteins are disclosed herein above and are known in the art.

VIII.D. Sequence Similarity and Identity

As used herein, the term “substantially similar” means that a particular sequence varies from nucleic acid sequence of SEQ ID NOs:1 or 3, or the amino acid sequence of SEQ ID NOs:2 or 4 by one or more deletions, substitutions, or additions, the net effect of which is to retain at least some of biological activity of the natural gene, gene product, or sequence. Such sequences include “mutant” or “polymorphic” sequences, or sequences in which the biological activity and/or the physical properties are altered to some degree but retains at least some or an enhanced degree of the original biological activity and/or physical properties. In determining nucleic acid sequences, all subject nucleic acid sequences capable of encoding substantially similar amino acid sequences are considered to be substantially similar to a reference nucleic acid sequence, regardless of differences in codon sequences or substitution of equivalent amino acids to create biologically functional equivalents.

VIII.D.1. Sequences that are Substantially Identical to an Engineered HNF4 or HNF4 LBD Mutant Sequence of the Present Invention

Nucleic acids that are substantially identical to a nucleic acid sequence of an engineered HNF4 or HNF4 LBD mutant of the present invention, e.g. allelic variants, genetically altered versions of the gene, etc., bind to an engineered HNF4 or HNF4 LBD mutant sequence under stringent hybridization conditions. By using probes, particularly labeled probes of DNA sequences, one can isolate homologous or related genes. The source of homologous genes can be any species, e.g. primate species; rodents, such as rats and mice, canines, felines, bovines, equines, yeast, nematodes, etc.

Between mammalian species, e.g. human and mouse, homologs have substantial sequence similarity, i.e. at least 75% sequence identity between nucleotide sequences. Sequence similarity is calculated based on a reference sequence, which can be a subset of a larger sequence, such as a conserved motif, coding region, flanking region, etc. A reference sequence will usually be at least about 18 nt long, more usually at least about 30 nt long, and can extend to the complete sequence that is being compared. Algorithms for sequence analysis are known in the art, such as BLAST, described in Altschul et al., (1990) J. Mol. Biol. 215: 403-10.

Percent identity or percent similarity of a DNA or peptide sequence can be determined, for example, by comparing sequence information using the GAP computer program, available from the University of Wisconsin Geneticist Computer Group. The GAP program utilizes the alignment method of Needleman et al., (1970) J. Mol. Biol. 48: 443, as revised by Smith et al., (1981) Adv. Appl. Math. 2:482. Briefly, the GAP program defines similarity as the number of aligned symbols (i.e., nucleotides or amino acids) which are similar, divided by the total number of symbols in the shorter of the two sequences. The preferred parameters for the GAP program are the default parameters, which do not impose a penalty for end gaps. See, e.g., Schwartz et al., eds., (1979), Atlas of Protein Sequence and Structure, National Biomedical Research Foundation, pp. 357-358, and Gribskov et al., (1986) Nucl. Acids. Res. 14: 6745.

The term “similarity” is contrasted with the term “identity”. Similarity is defined as above; “identity”, however, means a nucleic acid or amino acid sequence having the same amino acid at the same relative position in a given family member of a gene family. Homology and similarity are generally viewed as broader terms than the term identity. Biochemically similar amino acids, for example leucine/isoleucine or glutamate/aspartate, can be present at the same position-these are not identical per se, but are biochemically “similar.” As disclosed herein, these are referred to as conservative differences or conservative substitutions. This differs from a conservative mutation at the DNA level, which changes the nucleotide sequence without making a change in the encoded amino acid, e.g. TCC to TCA, both of which encode serine.

As used herein, DNA analog sequences are “substantially identical” to specific DNA sequences disclosed herein if: (a) the DNA analog sequence is derived from coding regions of the nucleic acid sequence shown in SEQ ID NOs:1 or 3; or (b) the DNA analog sequence is capable of hybridization with DNA sequences of (a) under stringent conditions and which encode a biologically active HNF4γ or HNF4γ LBD gene product; or (c) the DNA sequences are degenerate as a result of alternative genetic code to the DNA analog sequences defined in (a) and/or (b). Substantially identical analog proteins and nucleic acids will have between about 70% and 80%, preferably between about 81% to about 90% or even more preferably between about 91% and 99% sequence identity with the corresponding sequence of the native protein or nucleic acid. Sequences having lesser degrees of identity but comparable biological activity are considered to be equivalents.

As used herein, “stringent conditions” means conditions of high stringency, for example 6×SSC, 0.2% polyvinylpyrrolidone, 0.2% Ficoll, 0.2% bovine serum albumin, 0.1% sodium dodecyl sulfate, 100 μg/ml salmon sperm DNA and 15% formamide at 68° C. For the purposes of specifying additional conditions of high stringency, preferred conditions are salt concentration of about 200 mM and temperature of about 45° C. One example of such stringent conditions is hybridization at 4×SSC, at 65° C., followed by a washing in 0.1×SSC at 65° C. for one hour. Another exemplary stringent hybridization scheme uses 50% formamide, 4×SSC at 42° C.

In contrast, nucleic acids having sequence similarity are detected by hybridization under lower stringency conditions. Thus, sequence identity can be determined by hybridization under lower stringency conditions, for example, at 50° C. or higher and 0.1×SSC (9 mM NaCl/0.9 mM sodium citrate) and the sequences will remain bound when subjected to washing at 55° C. in 1×SSC.

VIII.D.2. Complementarity and Hybridization to an Engineered HNF4 or HNF4 LBD Mutant Sequence

As used herein, the term “complementary sequences” means nucleic acid sequences which are base-paired according to the standard Watson-Crick complementarity rules. The present invention also encompasses the use of nucleotide segments that are complementary to the sequences of the present invention.

Hybridization can also be used for assessing complementary sequences and/or isolating complementary nucleotide sequences. As discussed above, nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of about 30° C., typically in excess of about 37° C., and preferably in excess of about 45° C. Stringent salt conditions will ordinarily be less than about 1,000 mM, typically less than about 500 mM, and preferably less than about 200 mM. However, the combination of parameters is much more important than the measure of any single parameter. See, e.g., Wetmur & Davidson, (1968) J. Mol. Biol. 31: 349-70. Determining appropriate hybridization conditions to identify and/or isolate sequences containing high levels of homology is well known in the art. See, e.g., Sambrook et al., (1989) Molecular Cloning: A Laboratory Manual, Cold Spring Harbor, N.Y.

VIII.D.3. Functional Equivalents of an Engineered HNF4 or HNF4 LBD Mutant Nucleic Acid Sequence of the Present Invention

As used herein, the term “functionally equivalent codon” is used to refer to codons that encode the same amino acid, such as the ACG and AGU codons for serine. HNF4γ or HNF4γ LBD-encoding nucleic acid sequences comprising SEQ ID NOs:1 and 3 which have functionally equivalent codons are covered by the present invention. Thus, when referring to the sequence example presented in SEQ ID NOs:1 and 3, applicants contemplate substitution of functionally equivalent codons into the sequence example of SEQ ID NOs:1 and 3. Thus, applicants are in possession of amino acid and nucleic acids sequences which include such substitutions but which are not set forth herein in their entirety for convenience.

It will also be understood by those of skill in the art that amino acid and nucleic acid sequences can include additional residues, such as additional N- or C-terminal amino acids or 5′ or 3′ nucleic acid sequences, and yet still be essentially as set forth in one of the sequences disclosed herein, so long as the sequence retains biological protein activity where polypeptide expression is concerned. The addition of terminal sequences particularly applies to nucleic acid sequences which can, for example, include various non-coding sequences flanking either of the 5′ or 3′ portions of the coding region or can include various internal sequences, i.e., introns, which are known to occur within genes.

VIII.D.4. Biological Equivalents

The present invention envisions and includes biological equivalents of an engineered HNF4 or HNF4 LBD mutant polypeptide of the present invention. The term “biological equivalent” refers to proteins having amino acid sequences which are substantially identical to the amino acid sequence of an engineered HNF4 LBD mutant of the present invention and which are capable of exerting a biological effect in that they are capable of binding lipid moieties or cross-reacting with anti-HNF4 or HNF4 LBD mutant antibodies raised against an engineered mutant HNF4 or HNF4 LBD polypeptide of the present invention.

For example, certain amino acids can be substituted for other amino acids in a protein structure without appreciable loss of interactive capacity with, for example, structures in the nucleus of a cell. Since it is the interactive capacity and nature of a protein that defines that protein's biological functional activity, certain amino acid sequence substitutions can be made in a protein sequence (or the nucleic acid sequence encoding it) to obtain a protein with the same, enhanced, or antagonistic properties. Such properties can be achieved by interaction with the normal targets of the protein, but this need not be the case, and the biological activity of the invention is not limited to a particular mechanism of action. It is thus in accordance with the present invention that various changes can be made in the amino acid sequence of an engineered HNF4 or HNF4 LBD mutant polypeptide of the present invention or its underlying nucleic acid sequence without appreciable loss of biological utility or activity.

Biologically equivalent polypeptides, as used herein, are polypeptides in which certain, but not most or all, of the amino acids can be substituted. Thus, when referring to the sequence examples presented in SEQ ID NOs:1 and 3, applicants envision substitution of codons that encode biologically equivalent amino acids, as described herein, into the sequence example of SEQ ID NOs:2 and 4, respectively. Thus, applicants are in possession of amino acid and nucleic acids sequences which include such substitutions but which are not set forth herein in their entirety for convenience.

Alternatively, functionally equivalent proteins or peptides can be created via the application of recombinant DNA technology, in which changes in the protein structure can be engineered, based on considerations of the properties of the amino acids being exchanged, e.g. substitution of lie for Leu. Changes designed by man can be introduced through the application of site-directed mutagenesis techniques, e.g., to introduce improvements to the antigenicity of the protein or to test an engineered HNF4 or HNF4 LBD mutant polypeptide of the present invention in order to modulate lipid-binding or other activity, at the molecular level.

Amino acid substitutions, such as those which might be employed in modifying an engineered HNF4 or HNF4 LBD mutant polypeptide of the present invention are generally, but not necessarily, based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all of similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents. Other biologically functionally equivalent changes will be appreciated by those of skill in the art. It is implicit in the above discussion, however, that one of skill in the art can appreciate that a radical, rather than a conservative substitution is warranted in a given situation. Non-conservative substitutions in engineered mutant HNF4 or HNF4 LBD polypeptides of the present invention are also an aspect of the present invention.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte & Doolittle, (1982), J. Mol. Biol. 157: 105-132, incorporated herein by reference). It is known that certain amino acids can be substituted for other amino acids having a similar hydropathic index or score and still retain a similar biological activity. In making changes based upon the hydropathic index, the substitution of amino acids whose hydropathic indices are within +2 of the original value is preferred, those which are within +1 of the original value are particularly preferred, and those within ±0.5 of the original value are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 of the original value is preferred, those which are within ±1 of the original value are particularly preferred, and those within +0.5 of the original value are even more particularly preferred.

While discussion has focused on functionally equivalent polypeptides arising from amino acid changes, it will be appreciated that these changes can be effected by alteration of the encoding DNA, taking into consideration also that the genetic code is degenerate and that two or more codons can code for the same amino acid.

Thus, it will also be understood that this invention is not limited to the particular amino acid and nucleic acid sequences of SEQ ID NOs:1-4. Recombinant vectors and isolated DNA segments can therefore variously include an engineered HNF4γ or HNF4γ LBD mutant polypeptide-encoding region itself, include coding regions bearing selected alterations or modifications in the basic coding region, or include larger polypeptides which nevertheless comprise an HNF4γ or HNF4γ LBD mutant polypeptide-encoding regions or can encode biologically functional equivalent proteins or polypeptides which have variant amino acid sequences. Biological activity of an engineered HNF4γ or HNF4γ LBD mutant polypeptide can be determined, for example, by lipid-binding assays known to those of skill in the art.

The nucleic acid segments of the present invention, regardless of the length of the coding sequence itself, can be combined with other DNA sequences, such as promoters, enhancers, polyadenylation signals, additional restriction enzyme sites, multiple cloning sites, other coding segments, and the like, such that their overall length can vary considerably. It is therefore contemplated that a nucleic acid fragment of almost any length can be employed, with the total length preferably being limited by the ease of preparation and use in the intended recombinant DNA protocol. For example, nucleic acid fragments can be prepared which include a short stretch complementary to a nucleic acid sequence set forth in SEQ ID NOs:1 and 3, such as about 10 nucleotides, and which are up to 10,000 or 5,000 base pairs in length. DNA segments with total lengths of about 4,000, 3,000, 2,000, 1,000, 500, 200, 100, and about 50 base pairs in length are also useful.

The DNA segments of the present invention encompass biologically functional equivalents of engineered HNF4 or HNF4 LBD mutant polypeptides. Such sequences can rise as a consequence of codon redundancy and functional equivalency that are known to occur naturally within nucleic acid sequences and the proteins thus encoded. Alternatively, functionally equivalent proteins or polypeptides can be created via the application of recombinant DNA technology, in which changes in the protein structure can be engineered, based on considerations of the properties of the amino acids being exchanged. Changes can be introduced through the application of site-directed mutagenesis techniques, e.g., to introduce improvements to the antigenicity of the protein or to test variants of an engineered HNF4 or HNF4 LBD mutant of the present invention in order to examine the degree of lipid-binding activity, or other activity at the molecular level. Various site-directed mutagenesis techniques are known to those of skill in the art and can be employed in the present invention.

The invention further encompasses fusion proteins and peptides wherein an engineered HNF4 or HNF4 LBD mutant coding region of the present invention is aligned within the same expression unit with other proteins or peptides having desired functions, such as for purification or immunodetection purposes.

Recombinant vectors form important further aspects of the present invention. Particularly useful vectors are those in which the coding portion of the DNA segment is positioned under the control of a promoter. The promoter can be that naturally associated with an HNF4 gene, as can be obtained by isolating the 5′ non-coding sequences located upstream of the coding segment or exon, for example, using recombinant cloning and/or PCR technology and/or other methods known in the art, in conjunction with the compositions disclosed herein.

In other embodiments, certain advantages will be gained by positioning the coding DNA segment under the control of a recombinant, or heterologous, promoter. As used herein, a recombinant or heterologous promoter is a promoter that is not normally associated with an HNF4 gene in its natural environment. Such promoters can include promoters isolated from bacterial, viral, eukaryotic, or mammalian cells. Naturally, it will be important to employ a promoter that effectively directs the expression of the DNA segment in the cell type chosen for expression. The use of promoter and cell type combinations for protein expression is generally known to those of skill in the art of molecular biology (See, e.g., Sambrook et al., (1989) Molecular Cloning: A Laboratory Manual, Cold Spring Harbor Laboratory, New York, specifically incorporated herein by reference). The promoters employed can be constitutive or inducible and can be used under the appropriate conditions to direct high level expression of the introduced DNA segment, such as is advantageous in the large-scale production of recombinant proteins or peptides. One preferred promoter system contemplated for use in high-level expression is a T7 promoter-based system.

IX. The Role of the Three-Dimensional Structure of the HNF4γ LDB in Solving Additional HNF4 Crystals

Because polypeptides can crystallize in more than one crystal form, the structural coordinates of an HNF4γ LBD, or portions thereof, as provided by the present invention, are particularly useful in solving the structure of other crystal forms of HNF4γ and the crystalline forms of other HNF4s. The coordinates provided in the present invention can also be used to solve the structure of HNF4 or HNF4 LBD mutants (such as those described in Section VIII above), HNF4 LDB co-complexes, or of the crystalline form of any other protein with significant amino acid sequence homology to any functional domain of HNF4.

IX.A. Determining the Three-Dimensional Structure of a Polypeptide Using the Three-Dimensional Structure of the HNF4γ LBD as a Template in Molecular Replacement

One method that can be employed for the purpose of solving additional HNF4 crystal structures is molecular replacement. See generally, Rossmann, ed, (1972) The Molecular Replacement Method, Gordon & Breach, New York. In the molecular replacement method, the unknown crystal structure, whether it is another crystal form of an HNF4γ or an HNF4γ LBD, (i.e. an HNF4γ or an HNF4γ LBD mutant), or an HNF4γ or an HNF4γ LBD polypeptide complexed with another compound (a “co-complex”), or the crystal of some other protein with significant amino acid sequence homology to any functional region of the HNF4γ LBD, can be determined using the HNF4γ LBD structure coordinates provided in Table 2. This method provides an accurate structural form for the unknown crystal more quickly and efficiently than attempting to determine such information ab initio.

In addition, in accordance with this invention, HNF4γ or HNF4γ LBD mutants can be crystallized in complex with known modulators. The crystal structures of a series of such complexes can then be solved by molecular replacement and compared with that of wild-type HNF4γ or the wild-type HNF4γ LBD. Potential sites for modification within the various binding sites of the enzyme can thus be identified. This information provides an additional tool for determining the most efficient binding interactions, for example, increased hydrophobic interactions, between the HNF4γ LBD and a chemical entity or compound.

All of the complexes referred to in the present disclosure can be studied using X-ray diffraction techniques (See, e.g., Blundell & Johnson (1985) Method.Enzymol., 114A & 115B, (Wyckoff et al., eds.), Academic Press) and can be refined using computer software, such as the X-PLOR™ program (Brünger, (1992) X-PLOR, Version 3.1. A System for X-ray Crystallography and NMR, Yale University Press, New Haven, Conn.; X-PLOR is available from Molecular Simulations, Inc., San Diego, Calif.). This information can thus be used to optimize known classes of HNF4 and HNF4 LBD modulators, and more importantly, to design and synthesize novel classes of HNF4 and HNF4 LBD modulators.

### Laboratory Examples

The following Laboratory Examples have been included to illustrate preferred modes of the invention. Certain aspects of the following Laboratory Examples are described in terms of techniques and procedures found or contemplated by the present inventors to work well in the practice of the invention. These Laboratory Examples are exemplified through the use of standard laboratory practices of the inventors. In light of the present disclosure and the general level of skill in the art, those of skill will appreciate that the following Laboratory Examples are intended to be exemplary only and that numerous changes, modifications and alterations can be employed without departing from the spirit and scope of the invention.

### Laboratory Example 1

### Sub-Cloning and Protein Purification

Amino acids 102408 of the HNF4γ LDB (SEQ ID NO:3) were expressed by subcloning Into a T7 E. coli expression vector, pRSETa (Invitrogen, Carlsbad, Calif.). A histidine tag, sequence MKKGHHHHHHG (SEQ ID NO:5), was engineered at the N-terminus of the HNF4γ protein using a 5′ oligo. The plasmid was transformed into BL21 (DE3) cells which were grown at 22° C. overnight and were then harvested. The soluble protein was purified with an affinity column of Ni+2-NTA coupled agarose (Qiagen, Valencia, Calif.) (25 mM Tris pH=8.0, 50 mM imidazole pH=8.0, 150 mM NaCl). A 50-500 mM imidazole gradient was used for elution. HNF4γ eluted at 100 mM imidazole. The protein was diluted to 25 mM salt and further purified using a POROS™ 50HQ column (PerSeptive Biosystems, Foster City, Calif.) (25 mM Tris pH 8.0, 0.5 mM EDTA, 25 mM NaCl, 5 mM DTT, 5% Propane-diol) eluting with a 25 to 500 mM NaCl gradient. Two peaks were isolated, one representing homodimers of full-length HNF4γ LBD, the other containing heterodimers of full-length and C-terminally truncated HNF4γ. The homodimer peak was concentrated to 20 mg/ml and further purified by gel filtration chromatography (10 mM Tris pH 8.0, 0.1 mM EDTA, 150 mM NaCl, 10 mM DTT, 5% Propane-diol) using a Superdex 75 column (AP Biotech, Piscataway, N.J.). Protein sequence and purity were confirmed by N-terminal sequencing and mass spectrometry to greater than 95% homogeneity.

### Laboratory Example 2

### Crystallization

Crystallization trials were initially conducted with both the homogenous purified protein and the heterogeneous mixture. Crystals were obtained from both; however, the heterogeneous crystals were of poor diffraction quality. The purified protein was concentrated to 30 mg/ml (10 mM Tris pH 8.0, 0.1 mM EDTA, 150 mM NaCl, 10 mM DTT, 5% propane-diol) and crystallized using the vapor diffusion method by adding equal volume amounts of concentrated protein and a crystallization buffer of 0.75M ammonium di-hydrogen phosphate/di-ammonium hydrogen phosphate pH=5.0, 10 mM DTT. Crystals formed within 2-3 weeks and were suitable for data collection in 7 to 10 weeks.

### Laboratory Example 3

### Structure Determination and Refinement

HNF4γ LBD crystallized in the space group 14₁22 with a unit cell of dimensions a=b=152.71 Å, c=93.42 Å, α=β=γ=90°, and one molecule in the asymmetric unit. The structure was solved using single isomorphous replacement anomalous scattering (SIRAS) from a methyl-mercury derivative collected at beam line 171D at the Advanced Photon Source (located at the Argonne National Lab, Argonne, Ill.). Mercury sites were found using the software package Shake-and-Bake (Hauptman, (1997) Curr. Opin. Struct Biol. 7: 672-80; Weeks et al., (1993) Acta Cryst. D49: 179; available from the Hauptman-Woodward Medical Research Institute, Buffalo, N.Y.), and phases were improved by solvent flipping (Abrahams & Leslie, (1996) Acta Cryst. D52: 3042), which produced traceable electron density. Models were built using QUANTA™ (Molecular Simulations Inc., San Diego, Calif.), and refined using CNX™ (Molecular Simulations Inc., San Diego, Calif.).

### Laboratory Example 4

### GC/MS

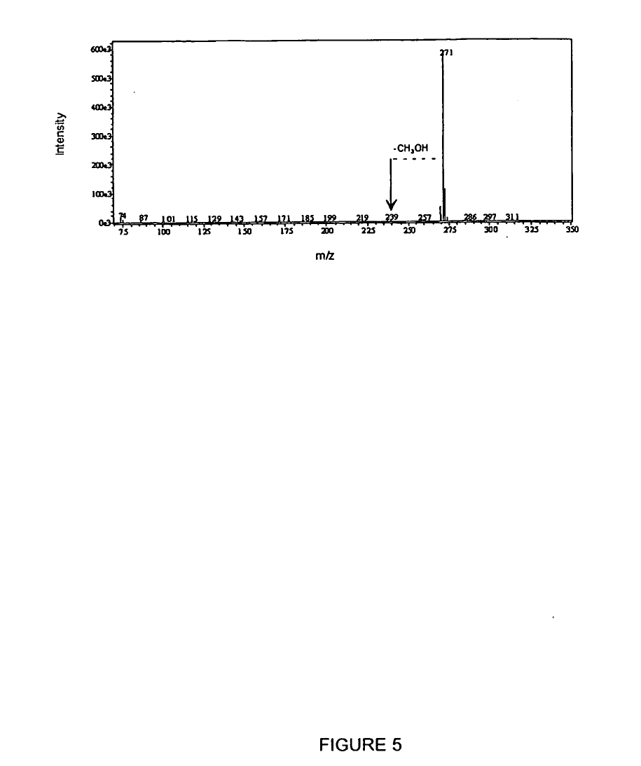

Lipids were extracted from an aliquot of HNF4γ LBD with chloroform/methanol 2:1 (v/v). The extract was dried under argon and then dissolved in a small volume of organic solvent. The extract was then treated with an aliquot of 3% (v/v) acetyl chloride in methanol for 30 min at room temperature to produce the methyl ester of the predicted fatty acid. After the reaction, the sample was dried again under argon. The derivatized sample was then analyzed by GC/MS on a Shimadzu GC-17A QP-5050A instrument. Analytes were eluted from a 25 meter DB5 column by increasing the column temperature from 100-280° C. at 120° C. per minute. Ionization of analytes was achieved by either EI or CI. Mass spectra were acquired using a scan range of 70-500 Da in 0.5 seconds. Representative data are depicted in FIGS. 4-7.

### Laboratory Example 5

### FRET Assay

A cell-free fluorescent resonance energy transfer (FRET) assay was used to measure the association between the amino portion of CBP (CREB-binding protein) (residues 54-457) and the HNF4 LBD (HNF4α amino acids 141465 and HNF4γ amino acids 102-408) (Zhou et al., (1998) Mol. Endocrinol 12: 1594-1604). Proteins were expressed in E. coli, purified to homogeneity, and biotinylated. CBP, the fluorescence donor, was labeled with a europium chelate, and HNF4 LBD was labeled with the streptavidin-conjugated fluorophore allophycocyanin (Molecular Probes, Eugene, Oreg.). Labeled HNF4 LBD and CBP were incubated together with ligands for 15 minutes at 21° C. before assaying. A small basal level was observed, as depicted in FIG. 3.

### Laboratory Example 6

### Computational Studies

The crystal structure of HNF4γ was subjected to hydrogen addition and subsequent minimization holding all heavy atoms fixed using the DISCOVER™ CVFF™ force field (Molecular Simulations, San Diego, Calif.). The model of palmitic acid was generated using the above-described HNF4γ protein and docking calculations using the program MVP (Lambert, (1997) in Practical Application of Computer-Aided Drug Design, (Charifson, ed.) Marcel-Dekker, New York, pp. 243-303). Crystallographically determined atoms were used as a template and the corresponding atoms in palmitic acid were constrained to within 0.5 Å of the template.

### REFERENCES

The references listed below as well as all references cited in the specification are incorporated herein by reference to the extent that they supplement, explain, provide a background for or teach methodology, techniques and/or compositions employed herein.
Abrahams & Leslie, (1996) Acta Cryst. D52: 30-42
Altschul et al., (1990) J. Mol. Biol. 215: 403-10
Ausubel et al., (1989) Current Protocols in Molecular Biology, Greene Publishing Associates and Wiley Interscience, New York
Bartlett et al., (1989) Special Pub., Royal Chem. Soc. 78: 182-96
Bjorklund & Grill (1999) Diabetes 48:1409-14
Blundell et al., (1976) Protein Crystallography, Academic Press
Blundell & Johnson, (1985) Method.Enzymol., 114A & 115B, (Wyckoff et al., eds.), Academic Press
Bogan et al., (1998) Nat. Struct. Biol. 5: 679-81
Bohm, (1992) J. Comput. Aid. Mol. Des., 6: 61-78
Bollheimer et al., (1998) J. Clin. Invest. 101:1094-1101
Bourguet et al., (1995) Nature 375: 377-82
Bourguet et al., (2000) Mol. Cell 5: 289-98
Brooks et al., (1983) J. Comp. Chem., 8: 132
Brünger, (1992) X-PLOR, Version 3.1. A System for X-ray Crystallography and NMR, Yale University Press, New Haven, Conn.
Brzozowski et al., (1997) Nature (London) 389: 753-58
Case et al., (1997), AMBER 5, University of California, San Francisco
Chan et al., (1993) Nucleic Acid Res. 21:1205-11
Cohen et al., (1990) J. Med. Chem. 33: 883-94
Creighton, (1983) Proteins: Structures and Molecular Principles, W.H. Freeman & Co., N.Y.
Dell & Hadzopoulou-Cladaras, (1999) J. Biol. Chem. 274: 9013-21
Diaz Guerra et al., (1993) Mol. Cell. Biol. 13:7725-33
Dobbins et al., (1998a) Diabetes 47:1613-18
Dobbins et al., (1998b) J. Clin. Invest. 101:2370-76
Drewes et al., (1996) Mol. Cell. Biol. 16:925-31
Ducruix & Geige, (1992) Crystallization of Nucleic Acids and Proteins: A Practical Approach, IRL Press, Oxford, England
Eisen et al., (1994) Proteins 19: 199-221
Folch et al., (1957) J. Biol. Chem. 226: 497-509
Gampe, Jr. et al., (2000) Mol. Cell 5: 545-55
Goodford, (1985) J. Med. Chem. 28: 849-57
Goodsell & Olsen, (1990) Proteins 8: 195-202
Gribskov et al., (1986) Nucl. Acids. Res. 14: 6745
Hauptman, (1997) Curr. Opin. Struct. Biol. 7: 672-80
Hertz et al., (1998) Nature (London) 392: 512-16
Jacqueminet et al., (2000) Metab. Clin. Exp. 49: 532-36
Jiang & Sladek, (1997) J. Biol. Chem. 272: 1218-25
Jiang et al., (1995) Mol. Cell. Biol. 15: 5131-43
Kuntz et al., (1992) J. Mol. Biol. 161: 269-88
Kyte & Doolittle, (1982), J. Mol. Biol. 157: 105-132
Lambert, (1997) in Practical Application of Computer-Aided Drug Design, (Charifson, ed.) Marcel-Dekker, New York, pp. 243-303
Lattman, (1985) Method Enzymol., 115: 55-77
Martin, (1992) J. Med. Chem. 35: 2145-54
McPherson, (1982) Preparation and Analysis of Protein Crystals, John Wiley, New York
McPherson, (1990) Eur. J. Biochem. 189:1-23
McPherson et al., (1989) Preparation and Analysis of Protein Crystals, Robert E. Krieger Publishing Company, Malabar, Fla.
McRee, (1992) J. Mol. Graphics 10: 44-47
Meng et al., (1992) J. Comput. Chem. 13: 505-24
Mietus-Snyder et al., (1992) Mol. Cell. Biol. 12:1708-18
Miquerol et al., (1994) J. Biol. Chem. 269:8944-51
Miranker & Karplus, (1991) Proteins 11: 29-34
Navia & Murcko, (1992) Curr. Opin. Struc. Biol. 2: 202-10
Needleman et al., (1970) J. Mol. Biol. 48: 443
Nicholls et al., (1991) Proteins 11: 282
Nishibata & Ital, (1991) Tetrahedron 47: 8985
Nolte et al., (1998) Nature (London) 395: 137-43
Otwinowski, (1991), in Isomorphous Replacement and Anomalous Scattering, (Evans & Leslie, eds.), pp. 80-86, Daresbury Laboratory, Daresbury, United Kingdom
Pearlman et al., (1995) Comput. Phys. Commun. 91: 1-41
Renaud et al., (1995) Nature 378: 681-89
Rossmann, ed, (1972) The Molecular Replacement Method, Gordon & Breach, New York
Sambrook et al., (1989) Molecular Cloning: A Laboratory Manual, Cold Spring Harbor Laboratory, New York
Schwartz et al., eds., (1979), Atlas of Protein Sequence and Structure, National Biomedical Research Foundation, pp. 357-358
Sheldrick (1990) Acta Cryst. A46: 467
Shih et al., (2000) Diabetes 49: 832-37
Sladek et al., Genes Dev. 4: 2353-65
Smith et al., (1981) Adv. Appl. Math. 2: 482
Stein et al., (1997) J. Clin. Invest. 100: 398-403
Stoffel & Duncan, (1997) Proc. Natl. Acad. Sci. U.S.A. 94: 13209-14
Van Holde, (1971) Physical Biochemistry, Prentice-Hall, N.J., 221-39
Wang et al., (1998) J. Biol. Chem. 273: 30847-50
Weber, (1991) Adv. Protein Chem. 41:1-36
Weeks et al., (1993) Acta Cryst. D49: 179
Wellner, (1971) Anal. Chem. 43: 597
Wetmur & Davidson, (1968) J. Mol. Biol. 31: 349-70
Xu et al., (1999) Mol. Cell 3: 397-403
Yamagata et al., (1996) Nature 384: 458-60
Zhou et al., (1998) Mol. Endocrinol 12: 1594-1604
Zhou & Grill, (1994) J. Clin. Invest. 93: 870-76
Zhou & Grill, (1995) J. Clin. Endocrinol Metab. 80: 1584-90
U.S. Pat. No. 4,554,101
U.S. Pat. No. 4,672,108
U.S. Pat. No. 4,833,233
U.S. Pat. No. 5,463,564
U.S. Pat. No. 6,008,033
WO 84/03564
WO 2000/037,077

WO 2000/025,134

TABLE 1
CRYSTALLOGRAPHIC DATA AND REFINEMENT

| | MeHgCl | Native |
|---|---|---|
| Crystal | | |
| Resolution Range | 40.-3.0 | 50.0-2.7 |
| Observations (Unique) | 413191 (10558) | 1308098 (18866) |
| Completeness | 99.6 (100) | 98.4 (93.7) |
| I/s | 36.3 (3.3) | 35.8 (3.5) |
| Rmerge % | 7.8 (44) | 4.8 (32) |
| Refinement Statistics | | |
| Resolution Range | | 50.0-2.7 |
| % Rfree | | 7 |
| Rcryst Rfree | | 24.8 (26.8) |
| Protein atoms | | 1774 |
| Ligand atoms | | 18 |
| Water Molecules | | 15 |
| Rmsd bonds/angles | | 0.0085/1.675 |
| Average Protein B factor | | 70.1 |

TABLE 2
ATOMIC STRUCTURE COORDINATE DATA OBTAINED FROM X-RAY
DIFFRACTION FROM THE LIGAND BINDING DOMAIN OF HNF4γ
COMPLEXED WITH PALMITIC ACID

| | ATOM | | PROTEIN | | | | | | |
|---|---|---|---|---|---|---|---|---|---|
| ATOM | TYPE | RESIDUE | # | # | X | Y | Z | OCC | B |
| 1 | CB | ALA | A | 99 | 45.376 | 107.876 | 27.936 | 1.00 | 95.38 |
| 2 | C | ALA | A | 99 | 44.561 | 106.474 | 29.858 | 1.00 | 94.20 |
| 3 | O | ALA | A | 99 | 45.642 | 106.159 | 30.365 | 1.00 | 94.42 |
| 4 | N | ALA | A | 99 | 44.646 | 108.963 | 30.044 | 1.00 | 95.61 |
| 5 | CA | ALA | A | 99 | 44.403 | 107.809 | 29.123 | 1.00 | 95.37 |
| 6 | N | ALA | A | 100 | 43.481 | 105.696 | 29.923 | 1.00 | 90.76 |
| 7 | CA | ALA | A | 100 | 43.519 | 104.399 | 30.596 | 1.00 | 87.68 |
| 8 | CB | ALA | A | 100 | 42.243 | 104.199 | 31.414 | 1.00 | 86.59 |
| 9 | C | ALA | A | 100 | 43.675 | 103.281 | 29.560 | 1.00 | 85.41 |
| 10 | O | ALA | A | 100 | 44.307 | 103.477 | 28.519 | 1.00 | 85.92 |
| 11 | N | GLY | A | 101 | 43.117 | 102.105 | 29.853 | 1.00 | 81.95 |
| 12 | CA | GLY | A | 101 | 43.187 | 101.004 | 28.907 | 1.00 | 75.36 |
| 13 | C | GLY | A | 101 | 44.054 | 99.816 | 29.268 | 1.00 | 71.65 |
| 14 | O | GLY | A | 101 | 44.002 | 98.788 | 28.592 | 1.00 | 71.37 |
| 15 | N | SER | A | 102 | 44.852 | 99.934 | 30.321 | 1.00 | 67.35 |
| 16 | CA | SER | A | 102 | 45.726 | 98.831 | 30.719 | 1.00 | 63.45 |
| 17 | CB | SER | A | 102 | 46.647 | 99.261 | 31.858 | 1.00 | 62.72 |
| 18 | OG | SER | A | 102 | 46.395 | 100.605 | 32.201 | 1.00 | 69.29 |
| 19 | C | SER | A | 102 | 44.919 | 97.623 | 31.154 | 1.00 | 60.14 |
| 20 | O | SER | A | 102 | 45.222 | 96.483 | 30.790 | 1.00 | 58.87 |
| 21 | N | ILE | A | 103 | 43.879 | 97.878 | 31.936 | 1.00 | 56.58 |
| 22 | CA | ILE | A | 103 | 43.040 | 96.809 | 32.429 | 1.00 | 54.03 |
| 23 | CB | ILE | A | 103 | 42.020 | 97.377 | 33.423 | 1.00 | 51.99 |
| 24 | CG2 | ILE | A | 103 | 40.987 | 96.312 | 33.822 | 1.00 | 46.51 |
| 25 | CG1 | ILE | A | 103 | 42.799 | 97.970 | 34.604 | 1.00 | 50.00 |
| 26 | CD1 | ILE | A | 103 | 42.480 | 97.390 | 35.953 | 1.00 | 48.75 |
| 27 | C | ILE | A | 103 | 42.349 | 96.067 | 31.291 | 1.00 | 54.95 |
| 28 | O | ILE | A | 103 | 42.157 | 94.847 | 31.366 | 1.00 | 55.77 |
| 29 | N | ASN | A | 104 | 41.997 | 96.791 | 30.232 | 1.00 | 54.74 |
| 30 | CA | ASN | A | 104 | 41.326 | 96.184 | 29.086 | 1.00 | 55.19 |
| 31 | CB | ASN | A | 104 | 40.777 | 97.257 | 28.143 | 1.00 | 55.87 |
| 32 | CG | ASN | A | 104 | 39.813 | 98.196 | 28.827 | 1.00 | 57.61 |
| 33 | OD1 | ASN | A | 104 | 40.169 | 98.892 | 29.787 | 1.00 | 59.08 |
| 34 | ND2 | ASN | A | 104 | 38.579 | 98.229 | 28.335 | 1.00 | 57.32 |
| 35 | C | ASN | A | 104 | 42.291 | 95.288 | 28.318 | 1.00 | 54.92 |
| 36 | O | ASN | A | 104 | 41.961 | 94.153 | 27.977 | 1.00 | 55.16 |
| 37 | N | THR | A | 105 | 43.486 | 95.804 | 28.047 | 1.00 | 54.02 |
| 38 | CA | THR | A | 105 | 44.488 | 95.039 | 27.319 | 1.00 | 53.20 |
| 39 | CB | THR | A | 105 | 45.788 | 95.819 | 27.171 | 1.00 | 55.37 |
| 40 | OG1 | THR | A | 105 | 45.532 | 97.048 | 26.481 | 1.00 | 57.24 |
| 41 | CG2 | THR | A | 105 | 46.807 | 94.989 | 26.403 | 1.00 | 54.39 |
| 42 | C | THR | A | 105 | 44.818 | 93.756 | 28.050 | 1.00 | 52.82 |
| 43 | O | THR | A | 105 | 44.861 | 92.687 | 27.451 | 1.00 | 53.56 |
| 44 | N | LEU | A | 106 | 45.062 | 93.862 | 29.349 | 1.00 | 50.68 |
| 45 | CA | LEU | A | 106 | 45.389 | 92.690 | 30.125 | 1.00 | 48.81 |
| 46 | CB | LEU | A | 106 | 45.758 | 93.100 | 31.557 | 1.00 | 47.15 |
| 47 | CG | LEU | A | 106 | 46.988 | 94.031 | 31.627 | 1.00 | 47.77 |
| 48 | CD1 | LEU | A | 106 | 47.315 | 94.387 | 33.071 | 1.00 | 47.75 |
| 49 | CD2 | LEU | A | 106 | 48.188 | 93.358 | 30.962 | 1.00 | 42.84 |
| 50 | C | LEU | A | 106 | 44.234 | 91.695 | 30.110 | 1.00 | 49.48 |
| 51 | O | LEU | A | 106 | 44.450 | 90.484 | 30.028 | 1.00 | 50.59 |
| 52 | N | ALA | A | 107 | 43.005 | 92.193 | 30.166 | 1.00 | 49.70 |
| 53 | CA | ALA | A | 107 | 41.846 | 91.303 | 30.176 | 1.00 | 50.54 |
| 54 | CB | ALA | A | 107 | 40.580 | 92.089 | 30.508 | 1.00 | 48.64 |
| 55 | C | ALA | A | 107 | 41.682 | 90.570 | 28.850 | 1.00 | 51.75 |
| 56 | O | ALA | A | 107 | 41.228 | 89.426 | 28.814 | 1.00 | 49.39 |
| 57 | N | GLN | A | 108 | 42.046 | 91.229 | 27.756 | 1.00 | 54.62 |
| 58 | CA | GLN | A | 108 | 41.921 | 90.600 | 26.457 | 1.00 | 57.26 |
| 59 | CB | GLN | A | 108 | 42.074 | 91.622 | 25.333 | 1.00 | 61.54 |
| 60 | CG | GLN | A | 108 | 41.327 | 91.193 | 24.084 | 1.00 | 71.90 |
| 61 | CD | GLN | A | 108 | 41.464 | 92.167 | 22.932 | 1.00 | 77.98 |
| 62 | OE1 | GLN | A | 108 | 40.643 | 92.175 | 22.001 | 1.00 | 82.40 |
| 63 | NE2 | GLN | A | 108 | 42.510 | 92.989 | 22.973 | 1.00 | 78.49 |
| 64 | C | GLN | A | 108 | 42.976 | 89.509 | 26.337 | 1.00 | 55.58 |
| 65 | O | GLN | A | 108 | 42.688 | 88.415 | 25.856 | 1.00 | 56.07 |
| 66 | N | ALA | A | 109 | 44.190 | 89.804 | 26.784 | 1.00 | 53.19 |
| 67 | CA | ALA | A | 109 | 45.266 | 88.831 | 26.742 | 1.00 | 52.55 |
| 68 | CB | ALA | A | 109 | 46.516 | 89.435 | 27.296 | 1.00 | 49.96 |
| 69 | C | ALA | A | 109 | 44.851 | 87.611 | 27.561 | 1.00 | 54.91 |
| 70 | O | ALA | A | 109 | 45.232 | 86.476 | 27.253 | 1.00 | 54.72 |
| 71 | N | GLU | A | 110 | 44.054 | 87.845 | 28.600 | 1.00 | 58.11 |
| 72 | CA | GLU | A | 110 | 43.568 | 86.755 | 29.450 | 1.00 | 62.24 |
| 73 | CB | GLU | A | 110 | 42.999 | 87.308 | 30.763 | 1.00 | 62.93 |
| 74 | CG | GLU | A | 110 | 44.052 | 87.811 | 31.732 | 1.00 | 68.89 |
| 75 | CD | GLU | A | 110 | 44.691 | 86.694 | 32.543 | 1.00 | 71.43 |
| 76 | OE1 | GLU | A | 110 | 45.754 | 86.942 | 33.152 | 1.00 | 72.25 |
| 77 | OE2 | GLU | A | 110 | 44.126 | 85.575 | 32.580 | 1.00 | 73.64 |
| 78 | C | GLU | A | 110 | 42.492 | 85.933 | 28.734 | 1.00 | 63.15 |
| 79 | O | GLU | A | 110 | 42.199 | 84.816 | 29.135 | 1.00 | 64.60 |
| 80 | N | VAL | A | 111 | 41.898 | 86.496 | 27.690 | 1.00 | 64.29 |
| 81 | CA | VAL | A | 111 | 40.870 | 85.798 | 26.930 | 1.00 | 66.07 |
| 82 | CB | VAL | A | 111 | 39.952 | 86.795 | 26.174 | 1.00 | 64.79 |
| 83 | CG1 | VAL | A | 111 | 39.009 | 86.050 | 25.248 | 1.00 | 62.48 |
| 84 | CG2 | VAL | A | 111 | 39.156 | 87.611 | 27.162 | 1.00 | 64.93 |
| 85 | C | VAL | A | 111 | 41.552 | 84.890 | 25.916 | 1.00 | 69.12 |
| 86 | O | VAL | A | 111 | 41.266 | 83.692 | 25.843 | 1.00 | 68.18 |
| 87 | N | ARG | A | 112 | 42.461 | 85.479 | 25.146 | 1.00 | 73.10 |
| 88 | CA | ARG | A | 112 | 43.204 | 84.762 | 24.122 | 1.00 | 76.92 |
| 89 | CB | ARG | A | 112 | 44.060 | 85.745 | 23.318 | 1.00 | 79.90 |
| 90 | CG | ARG | A | 112 | 43.264 | 86.903 | 22.704 | 1.00 | 84.65 |
| 91 | CD | ARG | A | 112 | 43.423 | 86.958 | 21.189 | 1.00 | 88.42 |
| 92 | NE | ARG | A | 112 | 44.657 | 87.616 | 20.762 | 1.00 | 91.17 |
| 93 | CZ | ARG | A | 112 | 45.423 | 87.189 | 19.759 | 1.00 | 93.20 |
| 94 | NH1 | ARG | A | 112 | 45.092 | 86.093 | 19.082 | 1.00 | 93.43 |
| 95 | NH2 | ARG | A | 112 | 46.509 | 87.871 | 19.422 | 1.00 | 94.12 |
| 96 | C | ARG | A | 112 | 44.079 | 83.667 | 24.725 | 1.00 | 78.59 |
| 97 | O | ARG | A | 112 | 44.341 | 82.654 | 24.077 | 1.00 | 79.07 |
| 98 | N | SER | A | 113 | 44.526 | 83.862 | 25.966 | 1.00 | 80.11 |
| 99 | CA | SER | A | 113 | 45.361 | 82.858 | 26.627 | 1.00 | 80.28 |
| 100 | CB | SER | A | 113 | 46.081 | 83.456 | 27.838 | 1.00 | 78.76 |
| 101 | OG | SER | A | 113 | 45.178 | 83.675 | 28.901 | 1.00 | 78.74 |
| 102 | C | SER | A | 113 | 44.524 | 81.660 | 27.077 | 1.00 | 80.80 |
| 103 | O | SER | A | 113 | 45.067 | 80.598 | 27.370 | 1.00 | 82.91 |
| 104 | N | ARG | A | 114 | 43.204 | 81.831 | 27.131 | 1.00 | 80.42 |
| 105 | CA | ARG | A | 114 | 42.308 | 80.751 | 27.532 | 1.00 | 80.13 |
| 106 | CB | ARG | A | 114 | 40.966 | 81.303 | 27.997 | 1.00 | 79.19 |
| 107 | CG | ARG | A | 114 | 40.966 | 81.970 | 29.349 | 1.00 | 78.23 |
| 108 | CD | ARG | A | 114 | 39.536 | 82.002 | 29.864 | 1.00 | 78.83 |
| 109 | NE | ARG | A | 114 | 39.356 | 82.815 | 31.061 | 1.00 | 79.39 |
| 110 | CZ | ARG | A | 114 | 39.216 | 84.137 | 31.054 | 1.00 | 79.53 |
| 111 | NH1 | ARG | A | 114 | 39.056 | 84.787 | 32.197 | 1.00 | 81.50 |
| 112 | NH2 | ARG | A | 114 | 39.226 | 84.810 | 29.913 | 1.00 | 78.80 |
| 113 | C | ARG | A | 114 | 42.058 | 79.742 | 26.407 | 1.00 | 81.14 |
| 114 | O | ARG | A | 114 | 41.591 | 78.634 | 26.655 | 1.00 | 79.80 |
| 115 | N | GLN | A | 115 | 42.335 | 80.137 | 25.170 | 1.00 | 83.68 |
| 116 | CA | GLN | A | 115 | 42.170 | 79.228 | 24.039 | 1.00 | 86.76 |
| 117 | CB | GLN | A | 115 | 41.785 | 79.987 | 22.768 | 1.00 | 86.56 |
| 118 | CG | GLN | A | 115 | 41.691 | 81.489 | 22.925 | 1.00 | 86.49 |
| 119 | CD | GLN | A | 115 | 41.601 | 82.202 | 21.588 | 1.00 | 86.47 |
| 120 | OE1 | GLN | A | 115 | 42.576 | 82.789 | 21.106 | 1.00 | 86.58 |
| 121 | NE2 | GLN | A | 115 | 40.429 | 82.143 | 20.973 | 1.00 | 85.92 |
| 122 | C | GLN | A | 115 | 43.540 | 78.595 | 23.855 | 1.00 | 88.96 |
| 123 | O | GLN | A | 115 | 44.478 | 79.252 | 23.397 | 1.00 | 89.14 |
| 124 | N | ILE | A | 116 | 43.664 | 77.322 | 24.214 | 1.00 | 91.44 |
| 125 | CA | ILE | A | 116 | 44.961 | 76.663 | 24.114 | 1.00 | 93.24 |
| 126 | CB | ILE | A | 116 | 45.815 | 77.000 | 25.352 | 1.00 | 92.35 |
| 127 | CG2 | ILE | A | 116 | 46.407 | 78.408 | 25.215 | 1.00 | 91.76 |
| 128 | CG1 | ILE | A | 116 | 44.958 | 76.822 | 26.614 | 1.00 | 90.28 |
| 129 | CD1 | ILE | A | 116 | 45.614 | 77.276 | 27.899 | 1.00 | 88.50 |
| 130 | C | ILE | A | 116 | 44.953 | 75.142 | 23.950 | 1.00 | 94.87 |
| 131 | O | ILE | A | 116 | 44.137 | 74.566 | 23.231 | 1.00 | 95.95 |
| 132 | N | SER | A | 117 | 45.893 | 74.511 | 24.638 | 1.00 | 95.66 |
| 133 | CA | SER | A | 117 | 46.062 | 73.071 | 24.596 | 1.00 | 95.95 |
| 134 | CB | SER | A | 117 | 47.294 | 72.737 | 23.747 | 1.00 | 95.95 |
| 135 | OG | SER | A | 117 | 48.419 | 73.495 | 24.172 | 1.00 | 95.95 |
| 136 | C | SER | A | 117 | 46.248 | 72.554 | 26.023 | 1.00 | 95.95 |
| 137 | O | SER | A | 117 | 45.361 | 72.706 | 26.865 | 1.00 | 95.95 |
| 138 | N | VAL | A | 118 | 47.412 | 71.954 | 26.270 | 1.00 | 95.90 |
| 139 | CA | VAL | A | 118 | 47.792 | 71.389 | 27.565 | 1.00 | 95.32 |
| 140 | CB | VAL | A | 118 | 47.852 | 72.499 | 28.672 | 1.00 | 94.33 |
| 141 | CG1 | VAL | A | 118 | 48.328 | 73.813 | 28.064 | 1.00 | 91.45 |
| 142 | CG2 | VAL | A | 118 | 46.502 | 72.652 | 29.373 | 1.00 | 93.04 |
| 143 | C | VAL | A | 118 | 46.901 | 70.223 | 28.043 | 1.00 | 95.95 |
| 144 | O | VAL | A | 118 | 45.680 | 70.206 | 27.752 | 1.00 | 95.95 |
| 145 | OXT | VAL | A | 118 | 47.452 | 69.328 | 28.728 | 1.00 | 95.95 |
| 146 | | VAL | A | 118 | | | | | |
| 147 | CB | ALA | A | 123 | 32.298 | 61.127 | 43.467 | 1.00 | 95.95 |
| 148 | C | ALA | A | 123 | 33.448 | 62.457 | 41.672 | 1.00 | 95.95 |
| 149 | O | ALA | A | 123 | 33.788 | 61.585 | 40.873 | 1.00 | 95.95 |
| 150 | N | ALA | A | 123 | 33.099 | 63.412 | 43.950 | 1.00 | 95.08 |
| 151 | CA | ALA | A | 123 | 33.385 | 62.169 | 43.180 | 1.00 | 95.95 |
| 152 | TF | SER | A | 124 | 33.172 | 63.699 | 41.296 | 1.00 | 95.95 |
| 153 | CA | SER | A | 124 | 33.119 | 64.108 | 39.891 | 1.00 | 95.75 |
| 154 | CB | SER | A | 124 | 31.683 | 64.531 | 39.618 | 1.00 | 95.95 |
| 155 | OG | SER | A | 124 | 31.143 | 65.129 | 40.796 | 1.00 | 95.95 |
| 156 | C | SER | A | 124 | 34.073 | 65.234 | 39.462 | 1.00 | 95.95 |
| 157 | O | SER | A | 124 | 35.130 | 64.999 | 38.866 | 1.00 | 95.95 |
| 158 | N | ALA | A | 125 | 33.629 | 66.462 | 39.727 | 1.00 | 95.95 |
| 159 | CA | ALA | A | 125 | 34.359 | 67.697 | 39.457 | 1.00 | 95.91 |
| 160 | CB | ALA | A | 125 | 33.465 | 68.685 | 38.715 | 1.00 | 95.50 |
| 161 | C | ALA | A | 125 | 34.581 | 68.142 | 40.896 | 1.00 | 95.95 |
| 162 | O | ALA | A | 125 | 34.035 | 69.145 | 41.371 | 1.00 | 95.06 |
| 163 | N | ASP | A | 126 | 35.368 | 67.317 | 41.584 | 1.00 | 95.95 |
| 164 | CA | ASP | A | 126 | 35.699 | 67.474 | 42.991 | 1.00 | 95.81 |
| 165 | CB | ASP | A | 126 | 35.100 | 66.296 | 43.759 | 1.00 | 95.13 |
| 166 | CG | ASP | A | 126 | 35.470 | 66.293 | 45.225 | 1.00 | 95.95 |
| 167 | OD1 | ASP | A | 126 | 36.511 | 66.878 | 45.601 | 1.00 | 95.95 |
| 168 | OD2 | ASP | A | 126 | 34.720 | 65.678 | 46.008 | 1.00 | 95.95 |
| 169 | C | ASP | A | 126 | 37.215 | 67.491 | 43.167 | 1.00 | 95.56 |
| 170 | O | ASP | A | 126 | 37.935 | 66.729 | 42.515 | 1.00 | 94.70 |
| 171 | N | ILE | A | 127 | 37.683 | 68.351 | 44.067 | 1.00 | 95.46 |
| 172 | CA | ILE | A | 127 | 39.107 | 68.493 | 44.333 | 1.00 | 95.63 |
| 173 | CB | ILE | A | 127 | 39.402 | 69.858 | 45.055 | 1.00 | 94.48 |
| 174 | CG2 | ILE | A | 127 | 39.107 | 69.765 | 46.548 | 1.00 | 93.67 |
| 175 | CG1 | ILE | A | 127 | 40.851 | 70.282 | 44.802 | 1.00 | 93.58 |
| 176 | CD1 | ILE | A | 127 | 41.086 | 70.811 | 43.398 | 1.00 | 91.76 |
| 177 | C | ILE | A | 127 | 39.686 | 67.330 | 45.149 | 1.00 | 95.95 |
| 178 | O | ILE | A | 127 | 40.595 | 66.642 | 44.691 | 1.00 | 95.95 |
| 179 | N | ASN | A | 128 | 39.144 | 67.091 | 46.339 | 1.00 | 95.95 |
| 180 | CA | ASN | A | 128 | 39.645 | 66.035 | 47.213 | 1.00 | 95.82 |
| 181 | CB | ASN | A | 128 | 38.997 | 66.187 | 48.592 | 1.00 | 95.95 |
| 182 | CG | ASN | A | 128 | 39.344 | 67.520 | 49.246 | 1.00 | 95.95 |
| 183 | OD1 | ASN | A | 128 | 38.486 | 68.396 | 49.404 | 1.00 | 95.95 |
| 184 | ND2 | ASN | A | 128 | 40.619 | 67.685 | 49.617 | 1.00 | 95.95 |
| 185 | C | ASN | A | 128 | 39.497 | 64.601 | 46.683 | 1.00 | 94.84 |
| 186 | O | ASN | A | 128 | 39.398 | 63.648 | 47.453 | 1.00 | 95.23 |
| 187 | N | VAL | A | 129 | 39.516 | 64.472 | 45.359 | 1.00 | 93.62 |
| 188 | CA | VAL | A | 129 | 39.396 | 63.200 | 44.638 | 1.00 | 92.16 |
| 189 | CB | VAL | A | 129 | 37.917 | 62.998 | 44.111 | 1.00 | 92.64 |
| 190 | CG1 | VAL | A | 129 | 37.872 | 62.181 | 42.829 | 1.00 | 91.97 |
| 191 | CG2 | VAL | A | 129 | 37.093 | 62.283 | 45.151 | 1.00 | 92.68 |
| 192 | C | VAL | A | 129 | 40.364 | 63.296 | 43.451 | 1.00 | 91.17 |
| 193 | O | VAL | A | 129 | 40.031 | 62.936 | 42.324 | 1.00 | 90.51 |
| 194 | N | LYS | A | 130 | 41.581 | 63.771 | 43.672 | 1.00 | 89.45 |
| 195 | CA | LYS | A | 130 | 42.430 | 63.896 | 42.500 | 1.00 | 87.77 |
| 196 | CB | LYS | A | 130 | 42.509 | 65.362 | 42.085 | 1.00 | 86.37 |
| 197 | CG | LYS | A | 130 | 41.177 | 66.057 | 42.202 | 1.00 | 82.54 |
| 198 | CD | LYS | A | 130 | 41.136 | 67.350 | 41.452 | 1.00 | 79.99 |
| 199 | CE | LYS | A | 130 | 40.993 | 67.065 | 39.984 | 1.00 | 78.70 |
| 200 | NZ | LYS | A | 130 | 40.183 | 68.120 | 39.330 | 1.00 | 77.13 |
| 201 | C | LYS | A | 130 | 43.814 | 63.299 | 42.488 | 1.00 | 87.56 |
| 202 | O | LYS | A | 130 | 44.472 | 63.142 | 43.518 | 1.00 | 87.42 |
| 203 | N | ALA | A | 131 | 44.232 | 62.968 | 41.273 | 1.00 | 87.33 |
| 204 | CA | ALA | A | 131 | 45.529 | 62.384 | 41.027 | 1.00 | 87.17 |
| 205 | CB | ALA | A | 131 | 45.812 | 62.359 | 39.525 | 1.00 | 86.74 |
| 206 | C | ALA | A | 131 | 46.549 | 63.244 | 41.751 | 1.00 | 86.91 |
| 207 | O | ALA | A | 131 | 46.804 | 64.381 | 41.358 | 1.00 | 87.23 |
| 208 | N | ILE | A | 132 | 47.103 | 62.708 | 42.834 | 1.00 | 86.12 |
| 209 | CA | ILE | A | 132 | 48.105 | 63.431 | 43.596 | 1.00 | 84.31 |
| 210 | CB | ILE | A | 132 | 48.341 | 62.773 | 44.960 | 1.00 | 82.19 |
| 211 | CG2 | ILE | A | 132 | 49.592 | 63.329 | 45.591 | 1.00 | 80.93 |
| 212 | CG1 | ILE | A | 132 | 47.116 | 63.017 | 45.849 | 1.00 | 81.34 |
| 213 | CD1 | ILE | A | 132 | 47.295 | 62.619 | 47.293 | 1.00 | 79.69 |
| 214 | C | ILE | A | 132 | 49.388 | 63.480 | 42.773 | 1.00 | 85.57 |
| 215 | O | ILE | A | 132 | 49.859 | 62.467 | 42.256 | 1.00 | 85.67 |
| 216 | N | ALA | A | 133 | 49.934 | 64.684 | 42.656 | 1.00 | 86.78 |
| 217 | CA | ALA | A | 133 | 51.124 | 64.955 | 41.859 | 1.00 | 86.55 |
| 218 | CB | ALA | A | 133 | 51.216 | 66.455 | 41.596 | 1.00 | 87.10 |
| 219 | C | ALA | A | 133 | 52.480 | 64.446 | 42.322 | 1.00 | 86.05 |
| 220 | O | ALA | A | 133 | 52.785 | 64.360 | 43.514 | 1.00 | 85.49 |
| 221 | N | SER | A | 134 | 53.294 | 64.141 | 41.319 | 1.00 | 85.85 |
| 222 | CA | SER | A | 134 | 54.651 | 63.648 | 41.480 | 1.00 | 85.47 |
| 223 | CB | SER | A | 134 | 54.836 | 62.369 | 40.667 | 1.00 | 86.54 |
| 224 | OG | SER | A | 134 | 54.405 | 62.563 | 39.326 | 1.00 | 85.37 |
| 225 | C | SER | A | 134 | 55.569 | 64.723 | 40.930 | 1.00 | 84.17 |
| 226 | O | SER | A | 134 | 55.111 | 65.648 | 40.265 | 1.00 | 84.65 |
| 227 | N | ILE | A | 135 | 56.861 | 64.591 | 41.192 | 1.00 | 82.68 |
| 228 | CA | ILE | A | 135 | 57.822 | 65.569 | 40.714 | 1.00 | 81.44 |
| 229 | CB | ILE | A | 135 | 59.241 | 65.123 | 41.049 | 1.00 | 81.17 |
| 230 | CG2 | ILE | A | 135 | 60.237 | 66.153 | 40.560 | 1.00 | 81.24 |
| 231 | CG1 | ILE | A | 135 | 59.360 | 64.918 | 42.559 | 1.00 | 81.55 |
| 232 | CD1 | ILE | A | 135 | 60.675 | 64.319 | 42.990 | 1.00 | 81.94 |
| 233 | C | ILE | A | 135 | 57.694 | 65.765 | 39.205 | 1.00 | 81.19 |
| 234 | O | ILE | A | 135 | 57.644 | 66.897 | 38.717 | 1.00 | 81.57 |
| 235 | N | GLY | A | 136 | 57.632 | 64.651 | 38.480 | 1.00 | 79.98 |
| 236 | CA | GLY | A | 136 | 57.515 | 64.697 | 37.032 | 1.00 | 78.08 |
| 237 | C | GLY | A | 136 | 56.169 | 65.234 | 36.607 | 1.00 | 77.36 |
| 238 | O | GLY | A | 136 | 56.030 | 65.806 | 35.523 | 1.00 | 77.19 |
| 239 | N | ASP | A | 137 | 55.160 | 65.030 | 37.446 | 1.00 | 77.11 |
| 240 | CA | ASP | A | 137 | 53.834 | 65.552 | 37.142 | 1.00 | 77.27 |
| 241 | CB | ASP | A | 137 | 52.807 | 65.111 | 38.190 | 1.00 | 78.06 |
| 242 | CG | ASP | A | 137 | 52.206 | 63.747 | 37.885 | 1.00 | 79.23 |
| 243 | OD1 | ASP | A | 137 | 52.224 | 63.351 | 36.700 | 1.00 | 79.32 |
| 244 | OD2 | ASP | A | 137 | 51.700 | 63.084 | 38.820 | 1.00 | 78.62 |
| 245 | C | ASP | A | 137 | 53.959 | 67.072 | 37.157 | 1.00 | 76.82 |
| 246 | O | ASP | A | 137 | 53.535 | 67.755 | 36.225 | 1.00 | 77.73 |
| 247 | N | VAL | A | 138 | 54.566 | 67.583 | 38.227 | 1.00 | 74.98 |
| 248 | CA | VAL | A | 138 | 54.790 | 69.011 | 38.407 | 1.00 | 72.09 |
| 249 | CB | VAL | A | 138 | 55.500 | 69.275 | 39.758 | 1.00 | 69.50 |
| 250 | CG1 | VAL | A | 138 | 56.007 | 70.698 | 39.823 | 1.00 | 68.92 |
| 251 | CG2 | VAL | A | 138 | 54.532 | 69.030 | 40.902 | 1.00 | 68.43 |
| 252 | C | VAL | A | 138 | 55.620 | 69.607 | 37.264 | 1.00 | 73.00 |
| 253 | O | VAL | A | 138 | 55.225 | 70.598 | 36.650 | 1.00 | 73.94 |
| 254 | N | CYS | A | 139 | 56.764 | 68.998 | 36.970 | 1.00 | 73.34 |
| 255 | CA | CYS | A | 139 | 57.640 | 69.487 | 35.910 | 1.00 | 73.46 |
| 256 | CB | CYS | A | 139 | 58.912 | 68.646 | 35.844 | 1.00 | 73.03 |
| 257 | SG | CYS | A | 139 | 59.882 | 68.672 | 37.361 | 1.00 | 75.79 |
| 258 | C | CYS | A | 139 | 56.991 | 69.518 | 34.534 | 1.00 | 73.93 |
| 259 | O | CYS | A | 139 | 57.506 | 70.158 | 33.622 | 1.00 | 73.62 |
| 260 | N | GLU | A | 140 | 55.878 | 68.821 | 34.367 | 1.00 | 75.40 |
| 261 | CA | GLU | A | 140 | 55.211 | 68.835 | 33.072 | 1.00 | 78.36 |
| 262 | CB | GLU | A | 140 | 54.348 | 67.585 | 32.881 | 1.00 | 82.20 |
| 263 | CG | GLU | A | 140 | 54.283 | 67.089 | 31.432 | 1.00 | 88.31 |
| 264 | CD | GLU | A | 140 | 55.670 | 66.768 | 30.843 | 1.00 | 93.87 |
| 265 | OE1 | GLU | A | 140 | 56.531 | 66.209 | 31.572 | 1.00 | 95.48 |
| 266 | OE2 | GLU | A | 140 | 55.893 | 67.063 | 29.643 | 1.00 | 95.89 |
| 267 | C | GLU | A | 140 | 54.347 | 70.090 | 33.021 | 1.00 | 77.74 |
| 268 | O | GLU | A | 140 | 54.432 | 70.866 | 32.062 | 1.00 | 78.09 |
| 269 | N | SER | A | 141 | 53.527 | 70.285 | 34.057 | 1.00 | 75.94 |
| 270 | CA | SER | A | 141 | 52.673 | 71.469 | 34.152 | 1.00 | 73.84 |
| 271 | CB | SER | A | 141 | 52.051 | 71.599 | 35.541 | 1.00 | 72.73 |
| 272 | OG | SER | A | 141 | 50.862 | 70.852 | 35.654 | 1.00 | 73.73 |
| 273 | C | SER | A | 141 | 53.536 | 72.689 | 33.910 | 1.00 | 73.29 |
| 274 | O | SER | A | 141 | 53.152 | 73.587 | 33.169 | 1.00 | 73.32 |
| 275 | N | MET | A | 142 | 54.707 | 72.710 | 34.538 | 1.00 | 71.76 |
| 276 | CA | MET | A | 142 | 55.613 | 73.829 | 34.382 | 1.00 | 71.52 |
| 277 | CB | MET | A | 142 | 56.916 | 73.573 | 35.136 | 1.00 | 69.70 |
| 278 | CG | MET | A | 142 | 56.762 | 73.592 | 36.651 | 1.00 | 70.49 |
| 279 | SD | MET | A | 142 | 58.341 | 73.522 | 37.548 | 1.00 | 71.68 |
| 280 | CE | MET | A | 142 | 57.787 | 73.490 | 39.227 | 1.00 | 72.18 |
| 281 | C | MET | A | 142 | 55.901 | 74.123 | 32.917 | 1.00 | 72.98 |
| 282 | O | MET | A | 142 | 55.819 | 75.280 | 32.488 | 1.00 | 74.62 |
| 283 | N | ALA | A | 143 | 56.223 | 73.091 | 32.140 | 1.00 | 73.11 |
| 284 | CA | ALA | A | 143 | 56.524 | 73.283 | 30.719 | 1.00 | 73.25 |
| 285 | CB | ALA | A | 143 | 57.026 | 71.985 | 30.098 | 1.00 | 72.62 |
| 286 | C | ALA | A | 143 | 55.298 | 73.781 | 29.966 | 1.00 | 73.43 |
| 287 | O | ALA | A | 143 | 55.389 | 74.701 | 29.147 | 1.00 | 73.18 |
| 288 | N | GLN | A | 144 | 54.154 | 73.158 | 30.238 | 1.00 | 73.15 |
| 289 | CA | GLN | A | 144 | 52.901 | 73.553 | 29.604 | 1.00 | 73.54 |
| 290 | CB | GLN | A | 144 | 51.719 | 72.800 | 30.234 | 1.00 | 75.06 |
| 291 | CG | GLN | A | 144 | 51.543 | 71.338 | 29.826 | 1.00 | 77.70 |
| 292 | CD | GLN | A | 144 | 50.398 | 70.643 | 30.581 | 1.00 | 79.20 |
| 293 | OE1 | GLN | A | 144 | 49.310 | 71.195 | 30.741 | 1.00 | 80.93 |
| 294 | NE2 | GLN | A | 144 | 50.648 | 69.424 | 31.037 | 1.00 | 80.45 |
| 295 | C | GLN | A | 144 | 52.705 | 75.052 | 29.838 | 1.00 | 72.67 |
| 296 | O | GLN | A | 144 | 52.608 | 75.848 | 28.901 | 1.00 | 73.16 |
| 297 | N | GLN | A | 145 | 52.670 | 75.415 | 31.117 | 1.00 | 70.73 |
| 298 | CA | GLN | A | 145 | 52.454 | 76.781 | 31.560 | 1.00 | 67.58 |
| 299 | CB | GLN | A | 145 | 52.344 | 76.797 | 33.077 | 1.00 | 65.85 |
| 300 | CG | GLN | A | 145 | 51.157 | 76.015 | 33.569 | 1.00 | 64.88 |
| 301 | CD | GLN | A | 145 | 49.858 | 76.610 | 33.090 | 1.00 | 66.60 |
| 302 | OE1 | GLN | A | 145 | 48.841 | 75.922 | 32.999 | 1.00 | 69.03 |
| 303 | NE2 | GLN | A | 145 | 49.876 | 77.906 | 32.793 | 1.00 | 66.18 |
| 304 | C | GLN | A | 145 | 53.480 | 77.792 | 31.101 | 1.00 | 67.27 |
| 305 | O | GLN | A | 145 | 53.203 | 78.990 | 31.096 | 1.00 | 67.02 |
| 306 | N | LEU | A | 146 | 54.664 | 77.325 | 30.721 | 1.00 | 67.13 |
| 307 | CA | LEU | A | 146 | 55.699 | 78.233 | 30.243 | 1.00 | 68.36 |
| 308 | CB | LEU | A | 146 | 57.086 | 77.617 | 30.419 | 1.00 | 68.70 |
| 309 | CG | LEU | A | 146 | 57.663 | 77.717 | 31.834 | 1.00 | 70.98 |
| 310 | CD1 | LEU | A | 146 | 58.943 | 76.891 | 31.938 | 1.00 | 69.66 |
| 311 | CD2 | LEU | A | 146 | 57.933 | 79.189 | 32.168 | 1.00 | 69.79 |
| 312 | C | LEU | A | 146 | 55.448 | 78.550 | 28.777 | 1.00 | 70.12 |
| 313 | O | LEU | A | 146 | 55.967 | 79.534 | 28.240 | 1.00 | 72.20 |
| 314 | N | LEU | A | 147 | 54.653 | 77.706 | 28.125 | 1.00 | 70.46 |
| 315 | CA | LEU | A | 147 | 54.309 | 77.928 | 26.728 | 1.00 | 70.71 |
| 316 | CB | LEU | A | 147 | 53.993 | 76.599 | 26.036 | 1.00 | 71.32 |
| 317 | CG | LEU | A | 147 | 55.209 | 75.680 | 25.849 | 1.00 | 72.36 |
| 318 | CD1 | LEU | A | 147 | 54.753 | 74.329 | 25.364 | 1.00 | 71.57 |
| 319 | CD2 | LEU | A | 147 | 56.196 | 76.295 | 24.860 | 1.00 | 72.32 |
| 320 | C | LEU | A | 147 | 53.100 | 78.857 | 26.729 | 1.00 | 69.91 |
| 321 | O | LEU | A | 147 | 52.978 | 79.734 | 25.870 | 1.00 | 71.07 |
| 322 | N | VAL | A | 148 | 52.217 | 78.669 | 27.708 | 1.00 | 67.53 |
| 323 | CA | VAL | A | 148 | 51.046 | 79.526 | 27.858 | 1.00 | 65.43 |
| 324 | CB | VAL | A | 148 | 50.229 | 79.165 | 29.114 | 1.00 | 64.44 |
| 325 | CG1 | VAL | A | 148 | 49.128 | 80.189 | 29.328 | 1.00 | 61.99 |
| 326 | CG2 | VAL | A | 148 | 49.634 | 77.780 | 28.972 | 1.00 | 64.58 |
| 327 | C | VAL | A | 148 | 51.591 | 80.937 | 28.040 | 1.00 | 65.14 |
| 328 | O | VAL | A | 148 | 51.013 | 81.911 | 27.563 | 1.00 | 65.26 |
| 329 | N | LEU | A | 149 | 52.721 | 81.020 | 28.736 | 1.00 | 64.53 |
| 330 | CA | LEU | A | 149 | 53.397 | 82.279 | 29.004 | 1.00 | 64.62 |
| 331 | CB | LEU | A | 149 | 54.642 | 82.029 | 29.860 | 1.00 | 62.67 |
| 332 | CO | LEU | A | 149 | 55.497 | 83.261 | 30.169 | 1.00 | 63.20 |
| 333 | CD1 | LEU | A | 149 | 54.716 | 84.234 | 31.040 | 1.00 | 64.08 |
| 334 | CD2 | LEU | A | 149 | 56.761 | 82.834 | 30.866 | 1.00 | 62.00 |
| 335 | C | LEU | A | 149 | 53.801 | 82.990 | 27.715 | 1.00 | 65.16 |
| 336 | O | LEU | A | 149 | 53.492 | 84.164 | 27.525 | 1.00 | 66.07 |
| 337 | N | VAL | A | 150 | 54.497 | 82.280 | 26.833 | 1.00 | 65.72 |
| 338 | CA | VAL | A | 150 | 54.941 | 82.859 | 25.568 | 1.00 | 65.84 |
| 339 | CB | VAL | A | 150 | 55.743 | 81.833 | 24.732 | 1.00 | 65.71 |
| 340 | CG1 | VAL | A | 150 | 56.208 | 82.473 | 23.443 | 1.00 | 64.98 |
| 341 | CG2 | VAL | A | 150 | 56.940 | 81.330 | 25.522 | 1.00 | 63.28 |
| 342 | C | VAL | A | 150 | 53.758 | 83.360 | 24.737 | 1.00 | 67.17 |
| 343 | O | VAL | A | 150 | 53.846 | 84.394 | 24.075 | 1.00 | 66.64 |
| 344 | N | GLU | A | 151 | 52.651 | 82.629 | 24.771 | 1.00 | 68.10 |
| 345 | CA | GLU | A | 151 | 51.477 | 83.040 | 24.021 | 1.00 | 70.10 |
| 346 | CB | GLU | A | 151 | 50.471 | 81.891 | 23.919 | 1.00 | 73.89 |
| 347 | CG | GLU | A | 151 | 50.815 | 80.889 | 22.807 | 1.00 | 80.45 |
| 348 | CD | GLU | A | 151 | 51.083 | 81.575 | 21.457 | 1.00 | 84.36 |
| 349 | OE1 | GLU | A | 151 | 50.187 | 82.3a5 | 20.960 | 1.00 | 85.05 |
| 350 | OE2 | GLU | A | 151 | 52.192 | 81.388 | 20.896 | 1.00 | 84.09 |
| 351 | C | GLU | A | 151 | 50.829 | 84.257 | 24.658 | 1.00 | 69.69 |
| 352 | O | GLU | A | 151 | 50.457 | 85.199 | 23.962 | 1.00 | 71.57 |
| 353 | N | TRP | A | 152 | 50.702 | 84.232 | 25.981 | 1.00 | 67.95 |
| 354 | CA | TRP | A | 152 | 50.113 | 85.336 | 26.735 | 1.00 | 65.17 |
| 355 | CB | TRP | A | 152 | 50.254 | 85.073 | 28.235 | 1.00 | 64.16 |
| 356 | CG | TRP | A | 152 | 49.864 | 86.229 | 29.117 | 1.00 | 63.25 |
| 357 | CD2 | TRP | A | 152 | 50.753 | 87.169 | 29.740 | 1.00 | 62.56 |
| 358 | CE2 | TRP | A | 152 | 49.957 | 88.053 | 30.496 | 1.00 | 62.55 |
| 359 | CE3 | TRP | A | 152 | 52.146 | 87.315 | 29.767 | 1.00 | 60.84 |
| 360 | CD1 | TRP | A | 152 | 48.602 | 86.596 | 29.484 | 1.00 | 62.18 |
| 361 | NE1 | TRP | A | 152 | 48.649 | 87.693 | 30.309 | 1.00 | 61.89 |
| 362 | CZ2 | TRP | A | 152 | 50.508 | 89.115 | 31.230 | 1.00 | 63.00 |
| 363 | CZ3 | TRP | A | 152 | 52.691 | 88.363 | 30.497 | 1.00 | 61.77 |
| 364 | CH2 | TRP | A | 152 | 51.873 | 89.238 | 31.235 | 1.00 | 62.39 |
| 365 | C | TRP | A | 152 | 50.807 | 86.647 | 26.394 | 1.00 | 64.30 |
| 366 | O | TRP | A | 152 | 50.153 | 87.631 | 26.066 | 1.00 | 63.52 |
| 367 | N | ALA | A | 153 | 52.133 | 86.648 | 26.481 | 1.00 | 63.82 |
| 368 | CA | ALA | A | 153 | 52.932 | 87.834 | 26.193 | 1.00 | 64.71 |
| 369 | CB | ALA | A | 153 | 54.420 | 87.512 | 26.341 | 1.00 | 61.96 |
| 370 | C | ALA | A | 153 | 52.651 | 88.383 | 24.801 | 1.00 | 66.54 |
| 371 | O | ALA | A | 153 | 52.554 | 89.590 | 24.618 | 1.00 | 65.93 |
| 372 | N | LYS | A | 154 | 52.523 | 87.488 | 23.823 | 1.00 | 70.74 |
| 373 | CA | LYS | A | 154 | 52.258 | 87.879 | 22.442 | 1.00 | 74.19 |
| 374 | CB | LYS | A | 154 | 52.267 | 86.652 | 21.536 | 1.00 | 76.20 |
| 375 | CG | LYS | A | 154 | 53.642 | 86.226 | 21.068 | 1.00 | 79.72 |
| 376 | CD | LYS | A | 154 | 53.520 | 85.062 | 20.095 | 1.00 | 83.94 |
| 377 | CE | LYS | A | 154 | 54.804 | 84.847 | 19.311 | 1.00 | 86.83 |
| 378 | NZ | LYS | A | 154 | 54.625 | 83.810 | 18.253 | 1.00 | 89.17 |
| 379 | C | LYS | A | 154 | 50.941 | 88.625 | 22.245 | 1.00 | 75.81 |
| 380 | O | LYS | A | 154 | 50.834 | 89.476 | 21.358 | 1.00 | 76.31 |
| 381 | N | TYR | A | 155 | 49.940 | 88.305 | 23.063 | 1.00 | 76.01 |
| 382 | CA | TYR | A | 155 | 48.645 | 88.957 | 22.952 | 1.00 | 76.70 |
| 383 | CB | TYR | A | 155 | 47.555 | 88.091 | 23.580 | 1.00 | 81.46 |
| 384 | CG | TYR | A | 155 | 47.524 | 86.668 | 23.057 | 1.00 | 87.69 |
| 385 | CD1 | TYR | A | 155 | 47.884 | 86.376 | 21.738 | 1.00 | 90.53 |
| 386 | CE1 | TYR | A | 155 | 47.817 | 85.067 | 21.240 | 1.00 | 93.30 |
| 387 | CD2 | TYR | A | 155 | 47.095 | 85.617 | 23.868 | 1.00 | 90.10 |
| 388 | CE2 | TYR | A | 155 | 47.020 | 84.309 | 23.381 | 1.00 | 92.95 |
| 389 | CZ | TYR | A | 155 | 47.379 | 84.041 | 22.067 | 1.00 | 93.86 |
| 390 | OH | TYR | A | 155 | 47.282 | 82.753 | 21.582 | 1.00 | 94.60 |
| 391 | C | TYR | A | 155 | 48.646 | 90.336 | 23.593 | 1.00 | 75.45 |
| 392 | O | TYR | A | 155 | 47.600 | 90.961 | 23.741 | 1.00 | 76.85 |
| 393 | N | ILE | A | 156 | 49.825 | 90.800 | 23.986 | 1.00 | 72.89 |
| 394 | CA | ILE | A | 156 | 49.976 | 92.123 | 24.571 | 1.00 | 71.65 |
| 395 | CB | ILE | A | 156 | 50.731 | 92.076 | 25.911 | 1.00 | 68.23 |
| 396 | CG2 | ILE | A | 156 | 51.109 | 93.481 | 26.331 | 1.00 | 66.06 |
| 397 | CG1 | ILE | A | 156 | 49.864 | 91.404 | 26.981 | 1.00 | 65.29 |
| 398 | CD1 | ILE | A | 156 | 50.620 | 90.992 | 28.221 | 1.00 | 59.40 |
| 399 | C | ILE | A | 156 | 50.807 | 92.906 | 23.562 | 1.00 | 75.04 |
| 400 | O | ILE | A | 156 | 52.015 | 92.690 | 23.443 | 1.00 | 75.84 |
| 401 | N | PRO | A | 157 | 50.166 | 93.826 | 22.820 | 1.00 | 76.73 |
| 402 | CD | PRO | A | 157 | 48.825 | 94.320 | 23.163 | 1.00 | 77.28 |
| 403 | CA | PRO | A | 157 | 50.767 | 94.682 | 21.787 | 1.00 | 78.12 |
| 404 | CG | PRO | A | 157 | 49.688 | 95.739 | 21.546 | 1.00 | 78.74 |
| 405 | CG | PRO | A | 157 | 48.957 | 95.784 | 22.854 | 1.00 | 78.01 |
| 406 | C | PRO | A | 157 | 52.116 | 95.307 | 22.120 | 1.00 | 78.74 |
| 407 | O | PRO | A | 157 | 53.092 | 95.130 | 21.385 | 1.00 | 78.83 |
| 408 | N | ALA | A | 158 | 52.167 | 96.048 | 23.221 | 1.00 | 79.14 |
| 409 | CA | ALA | A | 158 | 53.402 | 96.696 | 23.645 | 1.00 | 79.39 |
| 410 | CG | ALA | A | 158 | 53.204 | 97.311 | 25.027 | 1.00 | 78.10 |
| 411 | C | ALA | A | 158 | 54.593 | 95.716 | 23.647 | 1.00 | 79.63 |
| 412 | O | ALA | A | 158 | 55.737 | 96.111 | 23.416 | 1.00 | 81.18 |
| 413 | N | PHE | A | 159 | 54.320 | 94.440 | 23.897 | 1.00 | 78.10 |
| 414 | CA | PHE | A | 159 | 55.361 | 93.417 | 23.914 | 1.00 | 77.04 |
| 415 | CG | PHE | A | 159 | 54.830 | 92.149 | 24.589 | 1.00 | 74.93 |
| 416 | CG | PHE | A | 159 | 55.767 | 90.974 | 24.519 | 1.00 | 70.81 |
| 417 | CD1 | PHE | A | 159 | 56.866 | 90.892 | 25.365 | 1.00 | 70.05 |
| 418 | CD2 | PHE | A | 159 | 55.536 | 89.938 | 23.620 | 1.00 | 68.83 |
| 419 | CE1 | PHE | A | 159 | 57.718 | 89.789 | 25.320 | 1.00 | 69.21 |
| 420 | CE2 | PHE | A | 159 | 56.382 | 88.834 | 23.567 | 1.00 | 68.27 |
| 421 | CZ | PHE | A | 159 | 57.474 | 88.757 | 24.418 | 1.00 | 67.85 |
| 422 | C | PHE | A | 159 | 55.810 | 93.079 | 22.493 | 1.00 | 77.93 |
| 423 | O | PHE | A | 159 | 57.003 | 92.913 | 22.232 | 1.00 | 77.40 |
| 424 | N | CYS | A | 160 | 54.841 | 92.973 | 21.586 | 1.00 | 78.83 |
| 425 | CA | CYS | A | 160 | 55.103 | 92.640 | 20.189 | 1.00 | 80.28 |
| 426 | CB | CYS | A | 160 | 53.786 | 92.394 | 19.462 | 1.00 | 79.59 |
| 427 | SG | CYS | A | 160 | 52.902 | 90.944 | 20.065 | 1.00 | 82.03 |
| 428 | C | CYS | A | 160 | 55.902 | 93.694 | 19.443 | 1.00 | 81.71 |
| 429 | O | GYS | A | 160 | 56.650 | 93.372 | 18.522 | 1.00 | 81.61 |
| 430 | N | GLU | A | 161 | 55.744 | 94.949 | 19.848 | 1.00 | 83.80 |
| 431 | CA | GLU | A | 161 | 56.443 | 96.066 | 19.222 | 1.00 | 85.81 |
| 432 | CG | GLU | A | 161 | 55.666 | 97.355 | 19.484 | 1.00 | 87.23 |
| 433 | CG | GLU | A | 161 | 54.265 | 97.337 | 18.880 | 1.00 | 89.73 |
| 434 | CD | GLU | A | 161 | 53.343 | 98.367 | 19.511 | 1.00 | 91.91 |
| 435 | OE1 | GLU | A | 161 | 53.841 | 99.241 | 20.263 | 1.00 | 91.31 |
| 436 | OE2 | GLU | A | 161 | 52.121 | 98.301 | 19.252 | 1.00 | 92.34 |
| 437 | C | GLU | A | 161 | 57.881 | 96.206 | 19.713 | 1.00 | 86.38 |
| 438 | O | GLU | A | 161 | 58.630 | 97.075 | 19.251 | 1.00 | 86.35 |
| 439 | N | LEU | A | 162 | 58.259 | 95.347 | 20.656 | 1.00 | 86.57 |
| 440 | CA | LEU | A | 162 | 59.610 | 95.351 | 21.206 | 1.00 | 86.05 |
| 441 | CB | LEU | A | 162 | 59.651 | 94.677 | 22.579 | 1.00 | 84.95 |
| 442 | CG | LEU | A | 162 | 59.076 | 95.368 | 23.810 | 1.00 | 85.09 |
| 443 | CD1 | LEU | A | 162 | 59.210 | 94.427 | 24.993 | 1.00 | 84.65 |
| 444 | CD2 | LEU | A | 162 | 59.809 | 96.672 | 24.077 | 1.00 | 84.78 |
| 445 | C | LEU | A | 162 | 60.510 | 94.560 | 20.279 | 1.00 | 86.36 |
| 446 | O | LEU | A | 162 | 60.063 | 93.605 | 19.640 | 1.00 | 85.97 |
| 447 | N | PRO | A | 163 | 61.791 | 94.948 | 20.183 | 1.00 | 86.60 |
| 448 | CD | PRO | A | 163 | 62.516 | 96.098 | 20.745 | 1.00 | 86.06 |
| 449 | CA | PRO | A | 163 | 62.655 | 94.171 | 19.298 | 1.00 | 87.01 |
| 450 | CB | PRO | A | 163 | 64.000 | 94.896 | 19.389 | 1.00 | 85.41 |
| 451 | CG | PRO | A | 163 | 63.937 | 95.613 | 20.690 | 1.00 | 85.73 |
| 452 | C | PRO | A | 163 | 62.719 | 92.706 | 19.736 | 1.00 | 88.45 |
| 453 | O | PRO | A | 163 | 62.696 | 92.379 | 20.921 | 1.00 | 89.25 |
| 454 | N | LEU | A | 164 | 62.778 | 91.839 | 18.740 | 1.00 | 90.01 |
| 455 | CA | LEU | A | 164 | 62.826 | 90.391 | 18.887 | 1.00 | 91.31 |
| 456 | CB | LEU | A | 164 | 63.233 | 89.820 | 17.525 | 1.00 | 93.70 |
| 457 | CG | LEU | A | 164 | 62.566 | 90.662 | 16.414 | 1.00 | 95.67 |
| 458 | CD1 | LEU | A | 164 | 63.570 | 90.984 | 15.315 | 1.00 | 94.00 |
| 459 | CD2 | LEU | A | 164 | 61.322 | 89.942 | 15.873 | 1.00 | 95.69 |
| 460 | C | LEU | A | 164 | 63.711 | 89.815 | 20.007 | 1.00 | 91.13 |
| 461 | O | LEU | A | 164 | 63.551 | 88.657 | 20.386 | 1.00 | 91.42 |
| 462 | N | ASP | A | 165 | 64.632 | 90.613 | 20.539 | 1.00 | 90.81 |
| 463 | CA | ASP | A | 165 | 65.533 | 90.152 | 21.597 | 1.00 | 89.80 |
| 464 | CB | ASP | A | 165 | 66.941 | 90.690 | 21.359 | 1.00 | 92.39 |
| 465 | CG | ASP | A | 165 | 67.734 | 89.826 | 20.399 | 1.00 | 95.54 |
| 466 | OD1 | ASP | A | 165 | 67.104 | 89.026 | 19.667 | 1.00 | 95.95 |
| 467 | OD2 | ASP | A | 165 | 68.981 | 89.948 | 20.373 | 1.00 | 95.95 |
| 468 | C | ASP | A | 165 | 65.094 | 90.517 | 23.002 | 1.00 | 88.43 |
| 469 | O | ASP | A | 165 | 65.323 | 89.753 | 23.942 | 1.00 | 88.39 |
| 470 | N | ASP | A | 166 | 64.486 | 91.690 | 23.146 | 1.00 | 86.37 |
| 471 | CA | ASP | A | 166 | 64.001 | 92.144 | 24.443 | 1.00 | 83.98 |
| 472 | CB | ASP | A | 166 | 63.536 | 93.590 | 24.353 | 1.00 | 85.58 |
| 473 | CG | ASP | A | 166 | 64.673 | 94.543 | 24.086 | 1.00 | 86.69 |
| 474 | OD1 | ASP | A | 166 | 65.782 | 94.302 | 24.608 | 1.00 | 87.53 |
| 475 | OD2 | ASP | A | 166 | 64.456 | 95.541 | 23.372 | 1.00 | 87.88 |
| 476 | C | ASP | A | 166 | 62.845 | 91.255 | 24.873 | 1.00 | 81.98 |
| 477 | O | ASP | A | 166 | 62.640 | 90.997 | 26.063 | 1.00 | 81.09 |
| 478 | N | GLN | A | 167 | 62.091 | 90.796 | 23.883 | 1.00 | 79.33 |
| 479 | CA | GLN | A | 167 | 60.970 | 89.907 | 24.114 | 1.00 | 78.44 |
| 480 | CB | GLN | A | 167 | 60.374 | 89.465 | 22.784 | 1.00 | 78.73 |
| 481 | CG | GLN | A | 167 | 59.383 | 90.423 | 22.175 | 1.00 | 80.96 |
| 482 | CD | GLN | A | 167 | 58.908 | 89.948 | 20.816 | 1.00 | 81.58 |
| 483 | OE1 | GLN | A | 167 | 58.712 | 88.748 | 20.592 | 1.00 | 79.69 |
| 484 | NE2 | GLN | A | 167 | 58.711 | 90.890 | 19.900 | 1.00 | 82.38 |
| 485 | C | GLN | A | 167 | 61.468 | 88.670 | 24.844 | 1.00 | 77.98 |
| 486 | O | GLN | A | 167 | 60.789 | 88.122 | 25.715 | 1.00 | 78.61 |
| 487 | N | VAL | A | 168 | 62.662 | 88.237 | 24.470 | 1.00 | 76.37 |
| 488 | CA | VAL | A | 168 | 63.269 | 87.046 | 25.045 | 1.00 | 74.15 |
| 489 | CB | VAL | A | 168 | 64.367 | 86.498 | 24.106 | 1.00 | 75.65 |
| 490 | CG1 | VAL | A | 168 | 64.806 | 85.109 | 24.557 | 1.00 | 74.83 |
| 491 | CG2 | VAL | A | 168 | 63.837 | 86.456 | 22.673 | 1.00 | 76.04 |
| 492 | C | VAL | A | 168 | 63.855 | 87.274 | 26.432 | 1.00 | 71.12 |
| 493 | O | VAL | A | 168 | 63.831 | 86.375 | 27.269 | 1.00 | 70.68 |
| 494 | N | ALA | A | 169 | 64.377 | 88.468 | 26.676 | 1.00 | 68.41 |
| 495 | CA | ALA | A | 169 | 64.954 | 88.775 | 27.977 | 1.00 | 66.77 |
| 496 | CB | ALA | A | 169 | 65.694 | 90.092 | 27.925 | 1.00 | 64.43 |
| 497 | C | ALA | A | 169 | 63.855 | 88.829 | 29.026 | 1.00 | 66.98 |
| 498 | O | ALA | A | 169 | 64.006 | 88.277 | 30.116 | 1.00 | 67.68 |
| 499 | N | LEU | A | 170 | 62.744 | 89.485 | 28.694 | 1.00 | 66.54 |
| 500 | CA | LEU | A | 170 | 61.620 | 89.602 | 29.624 | 1.00 | 65.31 |
| 501 | CB | LEU | A | 170 | 60.506 | 90.466 | 29.026 | 1.00 | 62.20 |
| 502 | CG | LEU | A | 170 | 60.820 | 91.951 | 28.827 | 1.00 | 62.51 |
| 503 | CD1 | LEU | A | 170 | 59.628 | 92.632 | 28.170 | 1.00 | 61.74 |
| 504 | CD2 | LEU | A | 170 | 61.145 | 92.610 | 30.164 | 1.00 | 59.41 |
| 505 | C | LEU | A | 170 | 61.064 | 88.230 | 29.979 | 1.00 | 65.02 |
| 506 | O | LEU | A | 170 | 60.822 | 87.930 | 31.146 | 1.00 | 65.89 |
| 507 | N | LEU | A | 171 | 60.868 | 87.405 | 28.957 | 1.00 | 64.59 |
| 508 | CA | LEU | A | 171 | 60.344 | 86.060 | 29.124 | 1.00 | 64.70 |
| 509 | CB | LEU | A | 171 | 60.218 | 85.390 | 27.757 | 1.00 | 64.46 |
| 510 | CG | LEU | A | 171 | 59.102 | 85.867 | 26.824 | 1.00 | 64.80 |
| 511 | CD1 | LEU | A | 171 | 59.390 | 85.417 | 25.396 | 1.00 | 62.26 |
| 512 | CD2 | LEU | A | 171 | 57.765 | 85.319 | 27.314 | 1.00 | 62.74 |
| 513 | C | LEU | A | 171 | 61.217 | 85.191 | 30.029 | 1.00 | 66.21 |
| 514 | O | LEU | A | 171 | 60.713 | 84.389 | 30.811 | 1.00 | 65.95 |
| 515 | N | ARG | A | 172 | 62.530 | 85.355 | 29.923 | 1.00 | 67.44 |
| 516 | CA | ARG | A | 172 | 63.466 | 84.563 | 30.713 | 1.00 | 67.95 |
| 517 | CB | ARG | A | 172 | 64.790 | 84.415 | 29.950 | 1.00 | 73.51 |
| 518 | CG | ARG | A | 172 | 64.735 | 83.518 | 28.712 | 1.00 | 80.88 |
| 519 | CD | ARG | A | 172 | 66.080 | 83.504 | 27.971 | 1.00 | 86.71 |
| 520 | NE | ARG | A | 172 | 66.123 | 82.488 | 26.914 | 1.00 | 92.46 |
| 521 | CZ | ARG | A | 172 | 67.118 | 82.338 | 26.039 | 1.00 | 94.34 |
| 522 | NH1 | ARG | A | 172 | 68.179 | 83.142 | 26.075 | 1.00 | 93.95 |
| 523 | NH2 | ARG | A | 172 | 67.060 | 81.372 | 25.128 | 1.00 | 94.24 |
| 524 | C | ARG | A | 172 | 63.773 | 85.107 | 32.110 | 1.00 | 65.79 |
| 525 | O | ARG | A | 172 | 64.293 | 84.381 | 32.952 | 1.00 | 64.93 |
| 526 | N | ALA | A | 173 | 63.442 | 86.369 | 32.361 | 1.00 | 63.58 |
| 527 | CA | ALA | A | 173 | 63.750 | 86.997 | 33.643 | 1.00 | 61.78 |
| 528 | CB | ALA | A | 173 | 63.717 | 88.507 | 33.483 | 1.00 | 61.72 |
| 529 | C | ALA | A | 173 | 62.932 | 86.598 | 34.866 | 1.00 | 62.00 |
| 530 | O | ALA | A | 173 | 63.492 | 86.387 | 35.939 | 1.00 | 61.86 |
| 531 | N | HIS | A | 174 | 61.614 | 86.507 | 34.738 | 1.00 | 61.78 |
| 532 | CA | HIS | A | 174 | 60.821 | 86.150 | 35.902 | 1.00 | 60.73 |
| 533 | CB | HIS | A | 174 | 60.188 | 87.411 | 36.509 | 1.00 | 62.68 |
| 534 | CG | HIS | A | 174 | 61.172 | 88.510 | 36.771 | 1.00 | 62.85 |
| 535 | CD2 | HIS | A | 174 | 61.911 | 88.804 | 37.869 | 1.00 | 63.15 |
| 536 | ND1 | HIS | A | 174 | 61.523 | 89.443 | 35.815 | 1.00 | 63.25 |
| 537 | CE1 | HIS | A | 174 | 62.434 | 90.263 | 36.316 | 1.00 | 62.46 |
| 538 | NE2 | HIS | A | 174 | 62.686 | 89.896 | 37.556 | 1.00 | 63.69 |
| 539 | C | HIS | A | 174 | 59.760 | 85.101 | 35.619 | 1.00 | 60.12 |
| 540 | O | HIS | A | 174 | 58.695 | 85.086 | 36.241 | 1.00 | 60.90 |
| 541 | N | ALA | A | 175 | 60.062 | 84.213 | 34.678 | 1.00 | 59.27 |
| 542 | CA | ALA | A | 175 | 59.148 | 83.134 | 34.328 | 1.00 | 58.49 |
| 543 | CB | ALA | A | 175 | 59.810 | 82.201 | 33.327 | 1.00 | 57.98 |
| 544 | C | ALA | A | 175 | 58.757 | 82.361 | 35.590 | 1.00 | 58.48 |
| 545 | O | ALA | A | 175 | 57.643 | 81.846 | 35.690 | 1.00 | 57.69 |
| 546 | N | GLY | A | 176 | 59.682 | 82.290 | 36.550 | 1.00 | 59.08 |
| 547 | CA | GLY | A | 176 | 59.429 | 81.588 | 37.798 | 1.00 | 58.18 |
| 548 | C | GLY | A | 176 | 58.279 | 82.172 | 38.597 | 1.00 | 58.64 |
| 549 | O | GLY | A | 176 | 57.382 | 81.446 | 39.040 | 1.00 | 58.62 |
| 550 | N | GLU | A | 177 | 58.296 | 83.490 | 38.792 | 1.00 | 59.58 |
| 551 | CA | GLU | A | 177 | 57.234 | 84.153 | 39.538 | 1.00 | 60.16 |
| 552 | CB | GLU | A | 177 | 57.551 | 85.637 | 39.750 | 1.00 | 61.50 |
| 553 | CG | GLU | A | 177 | 58.622 | 85.903 | 40.794 | 1.00 | 63.08 |
| 554 | CD | GLU | A | 177 | 60.005 | 86.044 | 40.193 | 1.00 | 65.42 |
| 555 | OE1 | GLU | A | 177 | 60.336 | 85.293 | 39.249 | 1.00 | 65.86 |
| 556 | OE2 | GLU | A | 177 | 60.771 | 86.905 | 40.675 | 1.00 | 67.01 |
| 557 | C | GLU | A | 177 | 55.902 | 84.026 | 38.821 | 1.00 | 59.91 |
| 558 | O | GLU | A | 177 | 54.847 | 84.024 | 39.459 | 1.00 | 61.25 |
| 559 | N | HIS | A | 178 | 55.950 | 83.916 | 37.496 | 1.00 | 58.62 |
| 560 | CA | HIS | A | 178 | 54.734 | 83.785 | 36.693 | 1.00 | 57.01 |
| 561 | CB | HIS | A | 178 | 55.095 | 83.868 | 35.212 | 1.00 | 58.34 |
| 562 | CG | HIS | A | 178 | 53.994 | 84.387 | 34.350 | 1.00 | 63.30 |
| 563 | CD2 | HIS | A | 178 | 53.766 | 85.625 | 33.840 | 1.00 | 64.60 |
| 564 | ND1 | HIS | A | 178 | 52.947 | 83.601 | 33.909 | 1.00 | 64.96 |
| 565 | CE1 | HIS | A | 178 | 52.132 | 84.322 | 33.169 | 1.00 | 64.34 |
| 566 | NE2 | HIS | A | 178 | 52.605 | 85.556 | 33.110 | 1.00 | 66.45 |
| 567 | C | HIS | A | 178 | 54.030 | 82.458 | 37.007 | 1.00 | 55.43 |
| 568 | O | HIS | A | 178 | 52.809 | 82.399 | 37.192 | 1.00 | 54.25 |
| 569 | N | LEU | A | 179 | 54.810 | 81.391 | 37.080 | 1.00 | 52.78 |
| 570 | CA | LEU | A | 179 | 54.258 | 80.093 | 37.390 | 1.00 | 52.79 |
| 571 | CB | LEU | A | 179 | 55.351 | 79.022 | 37.278 | 1.00 | 52.58 |
| 572 | CG | LEU | A | 179 | 56.047 | 78.908 | 35.916 | 1.00 | 52.20 |
| 573 | CD1 | LEU | A | 179 | 57.180 | 77.879 | 35.988 | 1.00 | 49.88 |
| 574 | CD2 | LEU | A | 179 | 55.033 | 78.513 | 34.856 | 1.00 | 48.69 |
| 575 | C | LEU | A | 179 | 53.656 | 80.097 | 38.802 | 1.00 | 52.79 |
| 576 | O | LEU | A | 179 | 52.578 | 79.546 | 39.011 | 1.00 | 52.68 |
| 577 | N | LEU | A | 180 | 54.342 | 80.708 | 39.772 | 1.00 | 52.49 |
| 578 | CA | LEU | A | 180 | 53.815 | 80.744 | 41.141 | 1.00 | 50.34 |
| 579 | CB | LEU | A | 180 | 54.882 | 81.244 | 42.118 | 1.00 | 52.64 |
| 580 | CG | LEU | A | 180 | 56.040 | 80.271 | 42.413 | 1.00 | 54.04 |
| 581 | CD1 | LEU | A | 180 | 57.006 | 80.904 | 43.394 | 1.00 | 56.51 |
| 582 | CD2 | LEU | A | 180 | 55.515 | 78.973 | 42.992 | 1.00 | 53.85 |
| 583 | C | LEU | A | 180 | 52.531 | 81.581 | 41.248 | 1.00 | 47.53 |
| 584 | O | LEU | A | 180 | 51.591 | 81.195 | 41.938 | 1.00 | 45.44 |
| 585 | N | LEU | A | 181 | 52.476 | 82.716 | 40.565 | 1.00 | 45.83 |
| 586 | CA | LEU | A | 181 | 51.254 | 83.513 | 40.585 | 1.00 | 46.59 |
| 587 | CB | LEU | A | 181 | 51.415 | 84.767 | 39.732 | 1.00 | 45.48 |
| 588 | CG | LEU | A | 181 | 52.179 | 85.937 | 40.364 | 1.00 | 48.20 |
| 589 | CD1 | LEU | A | 181 | 52.559 | 86.933 | 39.290 | 1.00 | 46.83 |
| 590 | CD2 | LEU | A | 181 | 51.329 | 86.593 | 41.455 | 1.00 | 45.79 |
| 591 | C | LEU | A | 181 | 50.138 | 82.642 | 40.008 | 1.00 | 48.38 |
| 592 | O | LEU | A | 181 | 49.043 | 82.541 | 40.574 | 1.00 | 48.52 |
| 593 | N | GLY | A | 182 | 50.435 | 81.995 | 38.883 | 1.00 | 49.14 |
| 594 | CA | GLY | A | 182 | 49.460 | 81.132 | 38.236 | 1.00 | 47.78 |
| 595 | C | GLY | A | 182 | 48.912 | 80.020 | 39.110 | 1.00 | 48.81 |
| 596 | O | GLY | A | 182 | 47.704 | 79.822 | 39.184 | 1.00 | 51.08 |
| 597 | N | ALA | A | 183 | 49.790 | 79.283 | 39.778 | 1.00 | 49.60 |
| 598 | CA | ALA | A | 183 | 49.351 | 78.193 | 40.636 | 1.00 | 48.71 |
| 599 | CB | ALA | A | 183 | 50.542 | 77.366 | 41.066 | 1.00 | 47.94 |
| 600 | C | ALA | A | 183 | 48.599 | 78.719 | 41.854 | 1.00 | 49.37 |
| 601 | O | ALA | A | 183 | 47.647 | 78.086 | 42.323 | 1.00 | 49.24 |
| 602 | N | THR | A | 184 | 49.021 | 79.874 | 42.362 | 1.00 | 49.27 |
| 603 | CA | THR | A | 184 | 48.364 | 80.471 | 43.524 | 1.00 | 50.02 |
| 604 | CB | THR | A | 184 | 49.127 | 81.738 | 44.046 | 1.00 | 48.92 |
| 605 | OG1 | THR | A | 184 | 50.450 | 81.378 | 44.453 | 1.00 | 49.72 |
| 606 | CG2 | THR | A | 184 | 48.416 | 82.344 | 45.237 | 1.00 | 44.67 |
| 607 | C | THR | A | 184 | 46.949 | 80.889 | 43.136 | 1.00 | 51.11 |
| 608 | O | THR | A | 184 | 46.000 | 80.650 | 43.873 | 1.00 | 51.52 |
| 609 | N | LYS | A | 185 | 46.821 | 81.507 | 41.966 | 1.00 | 52.71 |
| 610 | CA | LYS | A | 185 | 45.535 | 81.994 | 41.476 | 1.00 | 55.79 |
| 611 | CB | LYS | A | 185 | 45.737 | 82.744 | 40.162 | 1.00 | 54.28 |
| 612 | CG | LYS | A | 185 | 44.877 | 83.970 | 40.004 | 1.00 | 57.75 |
| 613 | CD | LYS | A | 185 | 43.448 | 83.637 | 39.692 | 1.00 | 61.27 |
| 614 | CE | LYS | A | 185 | 42.624 | 84.908 | 39.558 | 1.00 | 60.47 |
| 615 | NZ | LYS | A | 185 | 41.268 | 84.579 | 39.044 | 1.00 | 61.67 |
| 616 | C | LYS | A | 185 | 44.539 | 80.862 | 41.262 | 1.00 | 57.83 |
| 617 | O | LYS | A | 185 | 43.353 | 80.984 | 41.595 | 1.00 | 59.99 |
| 618 | N | ARG | A | 186 | 45.038 | 79.763 | 40.714 | 1.00 | 57.58 |
| 619 | CA | ARG | A | 186 | 44.225 | 78.592 | 40.416 | 1.00 | 57.92 |
| 620 | CB | ARG | A | 186 | 45.022 | 77.691 | 39.465 | 1.00 | 57.27 |
| 621 | CG | ARG | A | 186 | 44.254 | 76.573 | 38.766 | 1.00 | 55.10 |
| 622 | CD | ARG | A | 186 | 45.059 | 76.144 | 37.553 | 1.00 | 54.62 |
| 623 | NE | ARG | A | 186 | 46.433 | 75.922 | 37.964 | 1.00 | 59.54 |
| 624 | CZ | ARG | A | 186 | 47.508 | 76.342 | 37.305 | 1.00 | 61.35 |
| 625 | NH1 | ARG | A | 186 | 47.381 | 77.022 | 36.173 | 1.00 | 59.99 |
| 626 | NH2 | ARG | A | 186 | 48.714 | 76.077 | 37.795 | 1.00 | 63.48 |
| 627 | C | ARG | A | 186 | 43.777 | 77.809 | 41.661 | 1.00 | 59.29 |
| 628 | O | ARG | A | 186 | 42.721 | 77.176 | 41.647 | 1.00 | 58.66 |
| 629 | N | SER | A | 187 | 44.565 | 77.870 | 42.736 | 1.00 | 59.78 |
| 630 | CA | SER | A | 187 | 44.264 | 77.144 | 43.970 | 1.00 | 60.64 |
| 631 | CB | SER | A | 187 | 45.562 | 76.650 | 44.593 | 1.00 | 60.64 |
| 632 | OG | SER | A | 187 | 46.368 | 76.015 | 43.621 | 1.00 | 60.45 |
| 633 | C | SER | A | 187 | 43.524 | 77.999 | 44.986 | 1.00 | 62.49 |
| 634 | O | SER | A | 187 | 42.971 | 77.506 | 45.965 | 1.00 | 63.57 |
| 635 | N | MET | A | 188 | 43.542 | 79.294 | 44.741 | 1.00 | 65.32 |
| 636 | CA | MET | A | 186 | 42.910 | 80.301 | 45.586 | 1.00 | 68.54 |
| 637 | CB | MET | A | 188 | 42.833 | 81.574 | 44.760 | 1.00 | 66.44 |
| 638 | CG | MET | A | 188 | 42.403 | 82.771 | 45.502 | 1.00 | 65.27 |
| 639 | SD | MET | A | 188 | 42.229 | 84.057 | 44.321 | 1.00 | 62.32 |
| 640 | CE | MET | A | 188 | 41.180 | 85.106 | 45.251 | 1.00 | 59.32 |
| 641 | C | MET | A | 188 | 41.508 | 79.955 | 46.139 | 1.00 | 71.36 |
| 642 | O | MET | A | 188 | 41.099 | 80.431 | 47.211 | 1.00 | 69.47 |
| 643 | N | MET | A | 189 | 40.785 | 79.134 | 45.383 | 1.00 | 74.71 |
| 644 | CA | MET | A | 189 | 39.420 | 78.720 | 45.703 | 1.00 | 76.70 |
| 645 | CB | MET | A | 189 | 38.694 | 78.441 | 44.398 | 1.00 | 77.78 |
| 646 | CG | MET | A | 189 | 39.637 | 77.947 | 43.327 | 1.00 | 81.52 |
| 647 | SD | MET | A | 189 | 39.408 | 78.872 | 41.822 | 1.00 | 91.12 |
| 648 | CE | MET | A | 189 | 40.266 | 77.807 | 40.590 | 1.00 | 87.46 |
| 649 | C | MET | A | 189 | 39.265 | 77.521 | 46.635 | 1.00 | 77.26 |
| 650 | O | MET | A | 189 | 38.156 | 77.238 | 47.090 | 1.00 | 77.40 |
| 651 | N | TYR | A | 190 | 40.361 | 76.823 | 46.926 | 1.00 | 77.11 |
| 652 | CA | TYR | A | 190 | 40.303 | 75.656 | 47.799 | 1.00 | 76.17 |
| 653 | CB | TYR | A | 190 | 41.098 | 74.521 | 47.173 | 1.00 | 74.05 |
| 654 | CG | TYR | A | 190 | 40.581 | 74.251 | 45.786 | 1.00 | 73.12 |
| 655 | CD1 | TYR | A | 190 | 39.311 | 73.715 | 45.593 | 1.00 | 73.09 |
| 656 | CE1 | TYR | A | 190 | 38.778 | 73.572 | 44.320 | 1.00 | 73.17 |
| 657 | CD2 | TYR | A | 190 | 41.313 | 74.628 | 44.663 | 1.00 | 71.70 |
| 658 | CE2 | TYR | A | 190 | 40.792 | 74.489 | 43.388 | 1.00 | 71.27 |
| 659 | CZ | TYR | A | 190 | 39.527 | 73.959 | 43.221 | 1.00 | 73.00 |
| 660 | OH | TYR | A | 190 | 39.006 | 73.831 | 41.954 | 1.00 | 75.02 |
| 661 | C | TYR | A | 190 | 40.765 | 75.967 | 49.205 | 1.00 | 77.18 |
| 662 | O | TYR | A | 190 | 41.391 | 76.993 | 49.445 | 1.00 | 75.10 |
| 663 | N | LYS | A | 191 | 40.459 | 75.072 | 50.136 | 1.00 | 80.68 |
| 664 | CA | LYS | A | 191 | 40.776 | 75.325 | 51.530 | 1.00 | 83.42 |
| 665 | CB | LYS | A | 191 | 39.512 | 75.083 | 52.376 | 1.00 | 87.13 |
| 666 | CG | LYS | A | 191 | 38.307 | 75.968 | 51.936 | 1.00 | 91.60 |
| 667 | CD | LYS | A | 191 | 38.669 | 77.476 | 51.979 | 1.00 | 92.10 |
| 668 | CE | LYS | A | 191 | 37.646 | 78.371 | 51.279 | 1.00 | 89.87 |
| 669 | NZ | LYS | A | 191 | 38.016 | 79.813 | 51.420 | 1.00 | 87.76 |
| 670 | C | LYS | A | 191 | 41.980 | 74.641 | 52.149 | 1.00 | 83.37 |
| 671 | O | LYS | A | 191 | 41.964 | 74.329 | 53.338 | 1.00 | 84.58 |
| 672 | N | ASP | A | 192 | 43.016 | 74.439 | 51.331 | 1.00 | 83.34 |
| 673 | CA | ASP | A | 192 | 44.312 | 73.858 | 51.726 | 1.00 | 82.85 |
| 674 | CB | ASP | A | 192 | 44.224 | 73.101 | 53.049 | 1.00 | 85.81 |
| 675 | CG | ASP | A | 192 | 45.302 | 73.527 | 54.024 | 1.00 | 88.14 |
| 676 | OD1 | ASP | A | 192 | 46.164 | 74.343 | 53.623 | 1.00 | 89.00 |
| 677 | OD2 | ASP | A | 192 | 45.291 | 73.054 | 55.182 | 1.00 | 89.49 |
| 678 | C | ASP | A | 192 | 44.938 | 72.947 | 50.678 | 1.00 | 81.27 |
| 679 | O | ASP | A | 192 | 45.730 | 72.057 | 50.995 | 1.00 | 79.20 |
| 680 | N | ILE | A | 193 | 44.597 | 73.197 | 49.420 | 1.00 | 80.44 |
| 681 | CA | ILE | A | 193 | 45.100 | 72.402 | 48.309 | 1.00 | 77.82 |
| 682 | CB | ILE | A | 193 | 43.944 | 71.677 | 47.563 | 1.00 | 78.91 |
| 683 | CG2 | ILE | A | 193 | 44.453 | 70.383 | 46.943 | 1.00 | 77.91 |
| 684 | CG1 | ILE | A | 193 | 42.766 | 71.420 | 48.515 | 1.00 | 80.16 |
| 685 | CD1 | ILE | A | 193 | 43.063 | 70.491 | 49.678 | 1.00 | 80.71 |
| 686 | C | ILE | A | 193 | 45.787 | 73.314 | 47.301 | 1.00 | 75.18 |
| 687 | O | ILE | A | 193 | 45.621 | 74.535 | 47.335 | 1.00 | 75.04 |
| 688 | N | LEU | A | 194 | 46.547 | 72.701 | 46.401 | 1.00 | 71.75 |
| 689 | CA | LEU | A | 194 | 47.250 | 73.415 | 45.351 | 1.00 | 68.94 |
| 690 | CB | LEU | A | 194 | 48.754 | 73.424 | 45.642 | 1.00 | 68.41 |
| 691 | CG | LEU | A | 194 | 49.481 | 74.774 | 45.610 | 1.00 | 67.08 |
| 692 | CD1 | LEU | A | 194 | 48.845 | 75.728 | 46.608 | 1.00 | 64.34 |
| 693 | CD2 | LEU | A | 194 | 50.958 | 74.577 | 45.927 | 1.00 | 65.33 |
| 694 | C | LEU | A | 194 | 46.950 | 72.668 | 44.049 | 1.00 | 68.41 |
| 695 | O | LEU | A | 194 | 47.372 | 71.531 | 43.860 | 1.00 | 67.07 |
| 696 | N | LEU | A | 195 | 46.203 | 73.312 | 43.158 | 1.00 | 67.56 |
| 697 | CA | LEU | A | 195 | 45.820 | 72.713 | 41.887 | 1.00 | 67.21 |
| 698 | CB | LEU | A | 195 | 44.372 | 73.097 | 41.577 | 1.00 | 67.35 |
| 699 | CG | LEU | A | 195 | 43.691 | 72.529 | 40.331 | 1.00 | 67.81 |
| 700 | CD1 | LEU | A | 195 | 43.537 | 71.022 | 40.453 | 1.00 | 68.28 |
| 701 | CD2 | LEU | A | 195 | 42.328 | 73.179 | 40.179 | 1.00 | 67.92 |
| 702 | C | LEU | A | 195 | 46.730 | 73.119 | 40.721 | 1.00 | 67.73 |
| 703 | O | LEU | A | 195 | 46.959 | 74.306 | 40.484 | 1.00 | 67.24 |
| 704 | N | LEU | A | 196 | 47.237 | 72.133 | 39.987 | 1.00 | 69.14 |
| 705 | CA | LEU | A | 196 | 48.115 | 72.402 | 38.851 | 1.00 | 70.88 |
| 706 | CB | LEU | A | 196 | 49.135 | 71.273 | 38.692 | 1.00 | 70.98 |
| 707 | CG | LEU | A | 196 | 49.964 | 70.916 | 39.928 | 1.00 | 71.86 |
| 708 | CD1 | LEU | A | 196 | 51.095 | 69.990 | 39.518 | 1.00 | 71.83 |
| 709 | CD2 | LEU | A | 196 | 50.529 | 72.178 | 40.565 | 1.00 | 71.12 |
| 710 | C | LEU | A | 196 | 47.342 | 72.584 | 37.541 | 1.00 | 72.68 |
| 711 | O | LEU | A | 196 | 46.141 | 72.305 | 37.466 | 1.00 | 72.73 |
| 712 | N | GLY | A | 197 | 48.044 | 73.052 | 36.509 | 1.00 | 74.33 |
| 713 | CA | GLY | A | 197 | 47.419 | 73.274 | 35.214 | 1.00 | 76.76 |
| 714 | C | GLY | A | 197 | 47.019 | 72.015 | 34.460 | 1.00 | 78.16 |
| 715 | O | GLY | A | 197 | 46.090 | 72.038 | 33.651 | 1.00 | 79.00 |
| 716 | N | ASN | A | 198 | 47.727 | 70.920 | 34.719 | 1.00 | 78.95 |
| 717 | CA | ASN | A | 198 | 47.452 | 69.634 | 34.081 | 1.00 | 77.71 |
| 718 | CB | ASN | A | 198 | 48.747 | 68.843 | 33.916 | 1.00 | 75.34 |
| 719 | CG | ASN | A | 198 | 49.438 | 68.590 | 35.235 | 1.00 | 73.85 |
| 720 | OD1 | ASN | A | 198 | 48.836 | 68.701 | 36.303 | 1.00 | 73.15 |
| 721 | ND2 | ASN | A | 198 | 50.709 | 68.236 | 35.166 | 1.00 | 74.53 |
| 722 | C | ASN | A | 198 | 46.462 | 68.823 | 34.917 | 1.00 | 78.19 |
| 723 | O | ASN | A | 198 | 46.275 | 67.630 | 34.697 | 1.00 | 78.86 |
| 724 | N | ASN | A | 199 | 45.861 | 69.481 | 35.899 | 1.00 | 78.85 |
| 725 | CA | ASN | A | 199 | 44.863 | 68.875 | 36.765 | 1.00 | 80.52 |
| 726 | CB | ASN | A | 199 | 43.827 | 68.149 | 35.907 | 1.00 | 81.74 |
| 727 | CG | ASN | A | 199 | 42.413 | 68.404 | 36.377 | 1.00 | 84.00 |
| 728 | OD1 | ASN | A | 199 | 41.994 | 67.924 | 37.430 | 1.00 | 85.46 |
| 729 | ND2 | ASN | A | 199 | 41.666 | 69.182 | 35.603 | 1.00 | 85.67 |
| 730 | C | ASN | A | 199 | 45.325 | 67.968 | 37.917 | 1.00 | 81.59 |
| 731 | O | ASN | A | 199 | 44.501 | 67.297 | 38.546 | 1.00 | 82.30 |
| 732 | N | TYR | A | 200 | 46.621 | 67.932 | 38.208 | 1.00 | 82.32 |
| 733 | CA | TYR | A | 200 | 47.085 | 67.127 | 39.340 | 1.00 | 82.86 |
| 734 | CB | TYR | A | 200 | 48.497 | 66.600 | 39.108 | 1.00 | 86.02 |
| 735 | CG | TYR | A | 200 | 48.553 | 65.434 | 38.148 | 1.00 | 90.33 |
| 736 | CD1 | TYR | A | 200 | 48.531 | 65.635 | 36.767 | 1.00 | 90.78 |
| 737 | CE1 | TYR | A | 200 | 48.591 | 64.563 | 35.880 | 1.00 | 91.68 |
| 738 | CD2 | TYR | A | 200 | 48.639 | 64.123 | 38.621 | 1.00 | 91.79 |
| 739 | CE2 | TYR | A | 200 | 48.697 | 63.044 | 37.743 | 1.00 | 92.89 |
| 740 | CZ | TYR | A | 200 | 48.677 | 63.273 | 36.371 | 1.00 | 92.47 |
| 741 | OH | TYR | A | 200 | 48.757 | 62.217 | 35.486 | 1.00 | 93.63 |
| 742 | C | TYR | A | 200 | 47.043 | 67.990 | 40.607 | 1.00 | 81.66 |
| 743 | O | TYR | A | 200 | 46.936 | 69.211 | 40.528 | 1.00 | 82.15 |
| 744 | N | VAL | A | 201 | 47.144 | 67.364 | 41.774 | 1.00 | 79.68 |
| 745 | CA | VAL | A | 201 | 47.057 | 68.101 | 43.035 | 1.00 | 76.53 |
| 746 | CB | VAL | A | 201 | 45.721 | 67.716 | 43.740 | 1.00 | 75.45 |
| 747 | CG1 | VAL | A | 201 | 45.860 | 67.737 | 45.248 | 1.00 | 75.91 |
| 748 | CG2 | VAL | A | 201 | 44.634 | 68.679 | 43.305 | 1.00 | 75.08 |
| 749 | C | VAL | A | 201 | 48.250 | 67.952 | 43.998 | 1.00 | 74.98 |
| 750 | O | VAL | A | 201 | 49.067 | 67.047 | 43.852 | 1.00 | 74.91 |
| 751 | N | ILE | A | 202 | 48.344 | 68.870 | 44.964 | 1.00 | 73.09 |
| 752 | CA | ILE | A | 202 | 49.408 | 68.883 | 45.972 | 1.00 | 71.49 |
| 753 | CB | ILE | A | 202 | 50.528 | 69.879 | 45.601 | 1.00 | 68.60 |
| 754 | CG2 | ILE | A | 202 | 51.565 | 69.925 | 46.706 | 1.00 | 67.15 |
| 755 | CG1 | ILE | A | 202 | 51.179 | 69.478 | 44.275 | 1.00 | 66.77 |
| 756 | CD1 | ILE | A | 202 | 52.307 | 70.397 | 43.834 | 1.00 | 60.49 |
| 757 | C | ILE | A | 202 | 48.838 | 69.323 | 47.322 | 1.00 | 73.37 |
| 758 | O | ILE | A | 202 | 48.490 | 70.490 | 47.489 | 1.00 | 73.27 |
| 759 | N | HIS | A | 203 | 48.754 | 68.404 | 48.286 | 1.00 | 75.88 |
| 760 | CA | HIS | A | 203 | 48.207 | 68.738 | 49.605 | 1.00 | 77.79 |
| 761 | CB | HIS | A | 203 | 47.600 | 67.498 | 50.289 | 1.00 | 80.65 |
| 762 | CG | HIS | A | 203 | 46.388 | 66.946 | 49.599 | 1.00 | 84.74 |
| 763 | CD2 | HIS | A | 203 | 45.068 | 67.069 | 49.875 | 1.00 | 86.58 |
| 764 | ND1 | HIS | A | 203 | 46.467 | 66.166 | 48.458 | 1.00 | 87.32 |
| 765 | CE1 | HIS | A | 203 | 45.247 | 65.837 | 48.068 | 1.00 | 87.56 |
| 766 | NE2 | HIS | A | 203 | 44.382 | 66.370 | 48.907 | 1.00 | 88.31 |
| 767 | C | HIS | A | 203 | 49.252 | 69.357 | 50.525 | 1.00 | 76.72 |
| 768 | O | HIS | A | 203 | 50.442 | 69.318 | 50.234 | 1.00 | 74.93 |
| 769 | N | ARG | A | 204 | 48.795 | 69.939 | 51.628 | 1.00 | 78.09 |
| 770 | CA | ARG | A | 204 | 49.699 | 70.560 | 52.590 | 1.00 | 80.70 |
| 771 | CB | ARG | A | 204 | 48.912 | 71.337 | 53.650 | 1.00 | 80.75 |
| 772 | CG | ARG | A | 204 | 49.775 | 72.295 | 54.466 | 1.00 | 81.75 |
| 773 | CD | ARG | A | 204 | 49.047 | 72.772 | 55.700 | 1.00 | 82.91 |
| 774 | NE | ARG | A | 204 | 48.520 | 74.129 | 55.589 | 1.00 | 86.56 |
| 775 | CZ | ARG | A | 204 | 49.238 | 75.231 | 55.780 | 1.00 | 88.77 |
| 776 | NH1 | ARG | A | 204 | 50.529 | 75.140 | 56.091 | 1.00 | 88.77 |
| 777 | NH2 | ARG | A | 204 | 48.659 | 76.424 | 55.685 | 1.00 | 89.41 |
| 778 | C | ARG | A | 204 | 50.531 | 69.471 | 53.264 | 1.00 | 82.55 |
| 779 | O | ARG | A | 204 | 50.037 | 68.360 | 53.501 | 1.00 | 83.37 |
| 780 | N | ASN | A | 205 | 51.789 | 69.787 | 53.576 | 1.00 | 83.50 |
| 781 | CA | ASN | A | 205 | 52.683 | 68.812 | 54.199 | 1.00 | 83.06 |
| 782 | CB | ASN | A | 205 | 52.220 | 68.507 | 55.624 | 1.00 | 81.52 |
| 783 | CG | ASN | A | 205 | 52.491 | 69.652 | 56.571 | 1.00 | 81.76 |
| 784 | OD1 | ASN | A | 205 | 51.885 | 69.757 | 57.632 | 1.00 | 82.84 |
| 785 | ND2 | ASN | A | 205 | 53.420 | 70.515 | 56.194 | 1.00 | 81.81 |
| 786 | C | ASN | A | 205 | 52.652 | 67.552 | 53.350 | 1.00 | 83.55 |
| 787 | O | ASN | A | 205 | 52.437 | 66.449 | 53.842 | 1.00 | 83.67 |
| 788 | N | SER | A | 206 | 52.860 | 67.739 | 52.054 | 1.00 | 84.99 |
| 789 | CA | SER | A | 206 | 52.835 | 66.635 | 51.118 | 1.00 | 87.22 |
| 790 | CB | SER | A | 206 | 53.233 | 67.124 | 49.724 | 1.00 | 87.83 |
| 791 | OG | SER | A | 206 | 53.159 | 66.072 | 48.776 | 1.00 | 90.49 |
| 792 | C | SER | A | 206 | 53.760 | 65.513 | 51.552 | 1.00 | 88.48 |
| 793 | O | SER | A | 206 | 54.777 | 65.744 | 52.206 | 1.00 | 88.05 |
| 794 | N | CYS | A | 207 | 53.383 | 64.289 | 51.198 | 1.00 | 90.80 |
| 795 | CA | CYS | A | 207 | 54.190 | 63.120 | 51.517 | 1.00 | 93.18 |
| 796 | CB | CYS | A | 207 | 53.380 | 61.838 | 51.291 | 1.00 | 93.25 |
| 797 | SG | CYS | A | 207 | 52.513 | 61.781 | 49.696 | 1.00 | 95.95 |
| 798 | C | CYS | A | 207 | 55.400 | 63.168 | 50.584 | 1.00 | 93.76 |
| 799 | O | CYS | A | 207 | 56.419 | 62.529 | 50.834 | 1.00 | 94.36 |
| 800 | N | GLU | A | 208 | 55.263 | 63.937 | 49.505 | 1.00 | 93.72 |
| 801 | CA | GLU | A | 208 | 56.324 | 64.129 | 48.520 | 1.00 | 93.46 |
| 802 | CB | GLU | A | 208 | 55.721 | 64.587 | 47.193 | 1.00 | 92.84 |
| 803 | CG | GLU | A | 208 | 55.415 | 63.488 | 46.208 | 1.00 | 94.58 |
| 804 | CD | GLU | A | 208 | 56.673 | 62.794 | 45.706 | 1.00 | 95.95 |
| 805 | OE1 | GLU | A | 208 | 57.752 | 63.439 | 45.705 | 1.00 | 95.95 |
| 806 | OE2 | GLU | A | 208 | 56.581 | 61.609 | 45.299 | 1.00 | 95.95 |
| 807 | C | GLU | A | 208 | 57.233 | 65.229 | 49.055 | 1.00 | 93.74 |
| 808 | O | GLU | A | 208 | 57.347 | 66.292 | 48.444 | 1.00 | 94.45 |
| 809 | N | VAL | A | 209 | 57.881 | 64.975 | 50.189 | 1.00 | 93.41 |
| 810 | CA | VAL | A | 209 | 58.737 | 65.978 | 50.823 | 1.00 | 93.12 |
| 811 | CB | VAL | A | 209 | 59.421 | 65.401 | 52.105 | 1.00 | 94.77 |
| 812 | CG1 | VAL | A | 209 | 58.356 | 64.842 | 53.057 | 1.00 | 93.37 |
| 813 | CG2 | VAL | A | 209 | 60.442 | 64.326 | 51.734 | 1.00 | 95.25 |
| 814 | C | VAL | A | 209 | 59.781 | 66.632 | 49.911 | 1.00 | 91.19 |
| 815 | O | VAL | A | 209 | 60.526 | 67.516 | 50.336 | 1.00 | 91.21 |
| 816 | N | GLU | A | 210 | 59.821 | 66.209 | 48.653 | 1.00 | 89.40 |
| 817 | CA | GLU | A | 210 | 60.740 | 66.780 | 47.676 | 1.00 | 88.37 |
| 818 | CB | GLU | A | 210 | 60.913 | 65.806 | 46.505 | 1.00 | 90.60 |
| 819 | CG | GLU | A | 210 | 61.879 | 66.250 | 45.407 | 1.00 | 93.35 |
| 820 | CD | GLU | A | 210 | 63.334 | 66.201 | 45.844 | 1.00 | 95.95 |
| 821 | OE1 | GLU | A | 210 | 63.653 | 65.418 | 46.767 | 1.00 | 95.95 |
| 822 | OE2 | GLU | A | 210 | 64.163 | 66.933 | 45.255 | 1.00 | 95.95 |
| 823 | C | GLU | A | 210 | 60.145 | 68.110 | 47.173 | 1.00 | 86.57 |
| 824 | O | GLU | A | 210 | 60.837 | 68.922 | 46.558 | 1.00 | 86.69 |
| 825 | N | ILE | A | 211 | 58.857 | 68.317 | 47.441 | 1.00 | 83.24 |
| 826 | CA | ILE | A | 211 | 58.150 | 69.529 | 47.025 | 1.00 | 79.90 |
| 827 | CB | ILE | A | 211 | 57.220 | 69.244 | 45.833 | 1.00 | 78.92 |
| 828 | CG2 | ILE | A | 211 | 57.991 | 68.533 | 44.734 | 1.00 | 78.06 |
| 829 | CG1 | ILE | A | 211 | 56.028 | 68.396 | 46.297 | 1.00 | 78.68 |
| 830 | CD1 | ILE | A | 211 | 55.086 | 67.974 | 45.184 | 1.00 | 75.93 |
| 831 | C | ILE | A | 211 | 57.296 | 70.095 | 48.168 | 1.00 | 78.80 |
| 832 | O | ILE | A | 211 | 56.613 | 71.107 | 48.012 | 1.00 | 78.27 |
| 833 | N | SER | A | 212 | 57.344 | 69.428 | 49.311 | 1.00 | 76.20 |
| 834 | CA | SER | A | 212 | 56.592 | 69.824 | 50.487 | 1.00 | 74.47 |
| 835 | CB | SER | A | 212 | 56.821 | 68.790 | 51.590 | 1.00 | 75.30 |
| 836 | OG | SER | A | 212 | 56.691 | 69.362 | 52.878 | 1.00 | 77.57 |
| 837 | C | SER | A | 212 | 56.908 | 71.216 | 51.024 | 1.00 | 73.37 |
| 838 | O | SER | A | 212 | 56.008 | 71.958 | 51.407 | 1.00 | 74.09 |
| 839 | N | ARG | A | 213 | 58.185 | 71.572 | 51.051 | 1.00 | 71.87 |
| 840 | CA | ARG | A | 213 | 58.609 | 72.863 | 51.587 | 1.00 | 70.38 |
| 841 | CB | ARG | A | 213 | 60.131 | 72.858 | 51.742 | 1.00 | 73.94 |
| 842 | CG | ARG | A | 213 | 60.639 | 73.470 | 53.022 | 1.00 | 78.32 |
| 843 | CD | ARG | A | 213 | 60.340 | 72.599 | 54.231 | 1.00 | 83.04 |
| 844 | NE | ARG | A | 213 | 61.155 | 73.026 | 55.369 | 1.00 | 89.48 |
| 845 | CZ | ARG | A | 213 | 60.955 | 72.653 | 56.629 | 1.00 | 91.42 |
| 846 | NH1 | ARG | A | 213 | 59.952 | 71.834 | 56.930 | 1.00 | 91.45 |
| 847 | NH2 | ARG | A | 213 | 61.759 | 73.108 | 57.589 | 1.00 | 91.27 |
| 848 | C | ARG | A | 213 | 58.168 | 74.081 | 50.762 | 1.00 | 67.58 |
| 849 | O | ARG | A | 213 | 57.608 | 75.032 | 51.302 | 1.00 | 67.01 |
| 850 | N | VAL | A | 214 | 58.427 | 74.061 | 49.460 | 1.00 | 64.81 |
| 851 | CA | VAL | A | 214 | 58.044 | 75.173 | 48.606 | 1.00 | 62.04 |
| 852 | CB | VAL | A | 214 | 58.607 | 74.996 | 47.184 | 1.00 | 58.84 |
| 853 | CG1 | VAL | A | 214 | 57.959 | 75.986 | 46.234 | 1.00 | 56.68 |
| 854 | CG2 | VAL | A | 214 | 60.102 | 75.220 | 47.207 | 1.00 | 57.41 |
| 855 | C | VAL | A | 214 | 56.525 | 75.307 | 48.546 | 1.00 | 63.00 |
| 856 | O | VAL | A | 214 | 55.985 | 76.408 | 48.678 | 1.00 | 63.30 |
| 857 | N | ALA | A | 215 | 55.835 | 74.184 | 48.362 | 1.00 | 62.03 |
| 858 | CA | ALA | A | 215 | 54.380 | 74.192 | 48.286 | 1.00 | 60.88 |
| 859 | CB | ALA | A | 215 | 53.871 | 72.800 | 47.981 | 1.00 | 59.03 |
| 860 | C | ALA | A | 215 | 53.726 | 74.723 | 49.564 | 1.00 | 61.63 |
| 861 | O | ALA | A | 215 | 52.703 | 75.414 | 49.504 | 1.00 | 61.65 |
| 862 | N | ASN | A | 216 | 54.301 | 74.399 | 50.721 | 1.00 | 60.68 |
| 863 | CA | ASN | A | 216 | 53.738 | 74.879 | 51.976 | 1.00 | 60.28 |
| 864 | CB | ASN | A | 216 | 54.419 | 74.228 | 53.184 | 1.00 | 61.56 |
| 865 | CG | ASN | A | 216 | 53.898 | 72.819 | 53.485 | 1.00 | 62.19 |
| 866 | OD1 | ASN | A | 216 | 54.265 | 72.236 | 54.497 | 1.00 | 63.73 |
| 867 | ND2 | ASN | A | 216 | 53.056 | 72.273 | 52.612 | 1.00 | 61.04 |
| 868 | C | ASN | A | 216 | 53.898 | 76.387 | 52.067 | 1.00 | 60.33 |
| 869 | O | ASN | A | 216 | 53.048 | 77.065 | 52.635 | 1.00 | 63.02 |
| 870 | N | ARG | A | 217 | 54.985 | 76.922 | 51.517 | 1.00 | 59.23 |
| 871 | CA | ARG | A | 217 | 55.192 | 78.370 | 51.558 | 1.00 | 58.81 |
| 872 | CB | ARG | A | 217 | 56.594 | 78.764 | 51.073 | 1.00 | 57.83 |
| 873 | CG | ARG | A | 217 | 57.714 | 78.526 | 52.077 | 1.00 | 57.69 |
| 874 | CD | ARG | A | 217 | 58.981 | 79.310 | 51.708 | 1.00 | 58.09 |
| 875 | NE | ARG | A | 217 | 58.831 | 80.753 | 51.908 | 1.00 | 55.60 |
| 876 | CZ | ARG | A | 217 | 59.725 | 81.659 | 51.515 | 1.00 | 55.82 |
| 877 | NH1 | ARG | A | 217 | 60.835 | 81.276 | 50.900 | 1.00 | 53.67 |
| 878 | NH2 | ARG | A | 217 | 59.512 | 82.955 | 51.738 | 1.00 | 55.80 |
| 879 | C | ARG | A | 217 | 54.163 | 79.035 | 50.670 | 1.00 | 59.19 |
| 880 | O | ARG | A | 217 | 53.542 | 80.020 | 51.064 | 1.00 | 60.54 |
| 881 | N | VAL | A | 218 | 53.994 | 78.491 | 49.467 | 1.00 | 58.88 |
| 882 | CA | VAL | A | 218 | 53.030 | 79.013 | 48.508 | 1.00 | 57.71 |
| 883 | CB | VAL | A | 218 | 52.951 | 78.117 | 47.259 | 1.00 | 57.51 |
| 884 | CG1 | VAL | A | 218 | 51.759 | 78.511 | 46.417 | 1.00 | 56.31 |
| 885 | CG2 | VAL | A | 218 | 54.254 | 78.234 | 46.444 | 1.00 | 56.52 |
| 886 | C | VAL | A | 218 | 51.663 | 79.081 | 49.165 | 1.00 | 58.31 |
| 887 | O | VAL | A | 218 | 50.958 | 80.079 | 49.045 | 1.00 | 59.36 |
| 888 | N | LEU | A | 219 | 51.301 | 78.019 | 49.874 | 1.00 | 59.03 |
| 889 | CA | LEU | A | 219 | 50.024 | 77.961 | 50.583 | 1.00 | 59.79 |
| 890 | CB | LEU | A | 219 | 49.804 | 76.566 | 51.169 | 1.00 | 58.97 |
| 891 | CG | LEU | A | 219 | 49.421 | 75.426 | 50.227 | 1.00 | 58.75 |
| 892 | CD1 | LEU | A | 219 | 49.467 | 74.100 | 50.964 | 1.00 | 58.97 |
| 893 | CD2 | LEU | A | 219 | 48.038 | 75.669 | 49.699 | 1.00 | 57.39 |
| 894 | C | LEU | A | 219 | 49.894 | 78.986 | 51.715 | 1.00 | 60.43 |
| 895 | O | LEU | A | 219 | 48.860 | 79.634 | 51.842 | 1.00 | 62.13 |
| 896 | N | ASP | A | 220 | 50.939 | 79.129 | 52.528 | 1.00 | 60.61 |
| 897 | CA | ASP | A | 220 | 50.930 | 80.046 | 53.673 | 1.00 | 60.83 |
| 898 | CB | ASP | A | 220 | 51.949 | 79.587 | 54.724 | 1.00 | 62.46 |
| 899 | CG | ASP | A | 220 | 51.589 | 78.255 | 55.345 | 1.00 | 66.34 |
| 900 | OD1 | ASP | A | 220 | 52.388 | 77.736 | 56.156 | 1.00 | 67.10 |
| 901 | OD2 | ASP | A | 220 | 50.509 | 77.724 | 55.026 | 1.00 | 70.35 |
| 902 | C | ASP | A | 220 | 51.213 | 81.515 | 53.383 | 1.00 | 60.01 |
| 903 | O | ASP | A | 220 | 50.628 | 82.410 | 54.006 | 1.00 | 59.81 |
| 904 | N | GLU | A | 221 | 52.117 | 81.762 | 52.443 | 1.00 | 57.85 |
| 905 | CA | GLU | A | 221 | 52.515 | 83.119 | 52.130 | 1.00 | 55.38 |
| 906 | CB | GLU | A | 221 | 54.038 | 83.176 | 52.087 | 1.00 | 55.40 |
| 907 | CG | GLU | A | 221 | 54.670 | 82.658 | 53.366 | 1.00 | 56.29 |
| 908 | CD | GLU | A | 221 | 56.173 | 82.532 | 53.269 | 1.00 | 59.30 |
| 909 | OE1 | GLU | A | 221 | 56.831 | 83.521 | 52.889 | 1.00 | 59.21 |
| 910 | OE2 | GLU | A | 221 | 56.704 | 81.443 | 53.575 | 1.00 | 61.34 |
| 911 | C | GLU | A | 221 | 51.930 | 83.725 | 50.871 | 1.00 | 53.92 |
| 912 | O | GLU | A | 221 | 52.113 | 84.910 | 50.629 | 1.00 | 52.66 |
| 913 | N | LEU | A | 222 | 51.228 | 82.933 | 50.069 | 1.00 | 53.81 |
| 914 | CA | LEU | A | 222 | 50.636 | 83.470 | 48.844 | 1.00 | 53.71 |
| 915 | CB | LEU | A | 222 | 51.347 | 82.888 | 47.616 | 1.00 | 51.30 |
| 916 | CG | LEU | A | 222 | 52.810 | 83.349 | 47.484 | 1.00 | 49.50 |
| 917 | CD1 | LEU | A | 222 | 53.461 | 82.687 | 46.285 | 1.00 | 47.29 |
| 918 | CD2 | LEU | A | 222 | 52.873 | 84.874 | 47.363 | 1.00 | 46.58 |
| 919 | C | LEU | A | 222 | 49.124 | 83.254 | 48.755 | 1.00 | 54.64 |
| 920 | O | LEU | A | 222 | 48.372 | 84.201 | 48.521 | 1.00 | 54.36 |
| 921 | N | VAL | A | 223 | 48.679 | 82.020 | 48.963 | 1.00 | 56.15 |
| 922 | CA | VAL | A | 223 | 47.254 | 81.713 | 48.908 | 1.00 | 57.59 |
| 923 | CB | VAL | A | 223 | 47.004 | 80.188 | 48.949 | 1.00 | 56.81 |
| 924 | CG1 | VAL | A | 223 | 45.518 | 79.914 | 49.038 | 1.00 | 54.89 |
| 925 | CG2 | VAL | A | 223 | 47.584 | 79.532 | 47.700 | 1.00 | 56.20 |
| 926 | C | VAL | A | 223 | 46.483 | 82.376 | 50.050 | 1.00 | 59.29 |
| 927 | O | VAL | A | 223 | 45.496 | 83.071 | 49.821 | 1.00 | 59.52 |
| 928 | N | ARG | A | 224 | 46.928 | 82.161 | 51.280 | 1.00 | 61.19 |
| 929 | CA | ARG | A | 224 | 46.263 | 82.761 | 52.426 | 1.00 | 62.98 |
| 930 | CB | ARG | A | 224 | 47.125 | 82.580 | 53.676 | 1.00 | 69.48 |
| 931 | CG | ARG | A | 224 | 46.506 | 83.093 | 54.965 | 1.00 | 77.90 |
| 932 | CD | ARG | A | 224 | 46.879 | 82.149 | 56.095 | 1.00 | 84.73 |
| 933 | NE | ARG | A | 224 | 46.527 | 80.776 | 55.724 | 1.00 | 90.58 |
| 934 | CZ | ARG | A | 224 | 46.763 | 79.698 | 56.466 | 1.00 | 92.62 |
| 935 | NH1 | ARG | A | 224 | 47.366 | 79.808 | 57.647 | 1.00 | 92.56 |
| 936 | NH2 | ARG | A | 224 | 46.388 | 78.501 | 56.024 | 1.00 | 92.21 |
| 937 | C | ARG | A | 224 | 46.015 | 84.246 | 52.156 | 1.00 | 60.60 |
| 938 | O | ARG | A | 224 | 44.877 | 84.707 | 52.194 | 1.00 | 60.88 |
| 939 | N | PRO | A | 225 | 47.079 | 85.011 | 51.866 | 1.00 | 58.53 |
| 940 | CD | PRO | A | 225 | 48.499 | 84.619 | 51.859 | 1.00 | 57.70 |
| 941 | CA | PRO | A | 225 | 46.942 | 86.444 | 51.586 | 1.00 | 57.18 |
| 942 | CB | PRO | A | 225 | 48.366 | 86.864 | 51.252 | 1.00 | 56.90 |
| 943 | CG | PRO | A | 225 | 49.192 | 85.936 | 52.074 | 1.00 | 56.41 |
| 944 | C | PRO | A | 225 | 45.983 | 86.726 | 50.432 | 1.00 | 57.76 |
| 945 | O | PRO | A | 225 | 45.213 | 87.694 | 50.479 | 1.00 | 57.84 |
| 946 | N | PHE | A | 226 | 46.047 | 85.891 | 49.389 | 1.00 | 56.60 |
| 947 | CA | PHE | A | 226 | 45.171 | 86.036 | 48.224 | 1.00 | 55.32 |
| 948 | CB | PHE | A | 226 | 45.503 | 84.973 | 47.159 | 1.00 | 53.08 |
| 949 | CG | PHE | A | 226 | 46.409 | 85.462 | 46.047 | 1.00 | 50.84 |
| 950 | CD1 | PHE | A | 226 | 47.580 | 86.162 | 46.328 | 1.00 | 49.31 |
| 951 | CD2 | PHE | A | 226 | 46.101 | 85.182 | 44.713 | 1.00 | 50.54 |
| 952 | CE1 | PHE | A | 226 | 48.436 | 86.575 | 45.298 | 1.00 | 49.01 |
| 953 | CE2 | PHE | A | 226 | 46.948 | 85.589 | 43.670 | 1.00 | 49.21 |
| 954 | CZ | PHE | A | 226 | 48.117 | 86.286 | 43.962 | 1.00 | 48.92 |
| 955 | C | PHE | A | 226 | 43.721 | 85.866 | 48.688 | 1.00 | 55.94 |
| 956 | O | PHE | A | 226 | 42.811 | 86.546 | 48.207 | 1.00 | 55.32 |
| 957 | N | GLN | A | 227 | 43.515 | 84.962 | 49.637 | 1.00 | 56.87 |
| 958 | CA | GLN | A | 227 | 42.184 | 84.710 | 50.162 | 1.00 | 58.58 |
| 959 | CB | GLN | A | 227 | 42.141 | 83.366 | 50.882 | 1.00 | 56.62 |
| 960 | CG | GLN | A | 227 | 42.638 | 82.221 | 50.040 | 1.00 | 59.05 |
| 961 | CD | GLN | A | 227 | 42.460 | 80.882 | 50.710 | 1.00 | 60.02 |
| 962 | OE1 | GLN | A | 227 | 42.872 | 80.680 | 51.855 | 1.00 | 61.01 |
| 963 | NE2 | GLN | A | 227 | 41.846 | 79.950 | 49.993 | 1.00 | 62.09 |
| 964 | C | GLN | A | 227 | 41.778 | 85.816 | 51.122 | 1.00 | 60.63 |
| 965 | O | GLN | A | 227 | 40.644 | 86.291 | 51.083 | 1.00 | 62.38 |
| 966 | N | GLU | A | 228 | 42.706 | 86.228 | 51.981 | 1.00 | 61.78 |
| 967 | CA | GLU | A | 228 | 42.416 | 87.277 | 52.946 | 1.00 | 63.03 |
| 968 | CB | GLU | A | 228 | 43.639 | 87.558 | 53.819 | 1.00 | 67.62 |
| 969 | CG | GLU | A | 228 | 44.010 | 86.404 | 54.747 | 1.00 | 76.28 |
| 970 | CD | GLU | A | 228 | 45.236 | 86.693 | 55.607 | 1.00 | 81.36 |
| 971 | OE1 | GLU | A | 228 | 46.328 | 86.972 | 55.045 | 1.00 | 84.37 |
| 972 | OE2 | GLU | A | 228 | 45.104 | 86.633 | 56.850 | 1.00 | 83.53 |
| 973 | C | GLU | A | 228 | 41.950 | 88.567 | 52.289 | 1.00 | 61.96 |
| 974 | O | GLU | A | 228 | 40.870 | 89.060 | 52.602 | 1.00 | 59.80 |
| 975 | N | ILE | A | 229 | 42.751 | 89.111 | 51.373 | 1.00 | 61.04 |
| 976 | CA | ILE | A | 229 | 42.384 | 90.357 | 50.706 | 1.00 | 61.78 |
| 977 | CG | ILE | A | 229 | 43.601 | 91.058 | 50.081 | 1.00 | 65.16 |
| 978 | CG2 | ILE | A | 229 | 44.834 | 90.889 | 50.961 | 1.00 | 67.55 |
| 979 | CG1 | ILE | A | 229 | 43.895 | 90.440 | 48.724 | 1.00 | 69.34 |
| 980 | CD1 | ILE | A | 229 | 44.960 | 91.157 | 47.982 | 1.00 | 72.97 |
| 981 | C | ILE | A | 229 | 41.370 | 90.174 | 49.584 | 1.00 | 60.67 |
| 982 | O | ILE | A | 229 | 40.820 | 91.156 | 49.077 | 1.00 | 59.92 |
| 983 | N | GLN | A | 230 | 41.146 | 88.924 | 49.182 | 1.00 | 59.30 |
| 984 | CA | GLN | A | 230 | 40.210 | 88.607 | 48.105 | 1.00 | 59.64 |
| 985 | CG | GLN | A | 230 | 38.769 | 88.908 | 48.550 | 1.00 | 61.87 |
| 986 | CG | GLN | A | 230 | 38.115 | 87.759 | 49.324 | 1.00 | 67.60 |
| 987 | CD | GLN | A | 230 | 36.654 | 88.032 | 49.684 | 1.00 | 72.09 |
| 988 | OE1 | GLN | A | 230 | 35.951 | 87.150 | 50.187 | 1.00 | 74.46 |
| 989 | NE2 | GLN | A | 230 | 36.195 | 89.256 | 49.429 | 1.00 | 71.65 |
| 990 | C | GLN | A | 230 | 40.529 | 89.334 | 46.789 | 1.00 | 57.13 |
| 991 | O | GLN | A | 230 | 39.721 | 90.095 | 46.264 | 1.00 | 56.69 |
| 992 | N | ILE | A | 231 | 41.713 | 89.073 | 46.251 | 1.00 | 54.06 |
| 993 | CA | ILE | A | 231 | 42.160 | 89.707 | 45.013 | 1.00 | 52.71 |
| 994 | CB | ILE | A | 231 | 43.662 | 89.394 | 44.795 | 1.00 | 50.87 |
| 995 | CG2 | ILE | A | 231 | 43.873 | 87.898 | 44.727 | 1.00 | 51.30 |
| 996 | CG1 | ILE | A | 231 | 44.162 | 90.048 | 43.513 | 1.00 | 51.63 |
| 997 | CD1 | ILE | A | 231 | 45.615 | 89.747 | 43.218 | 1.00 | 51.05 |
| 998 | C | ILE | A | 231 | 41.340 | 89.281 | 43.770 | 1.00 | 51.08 |
| 999 | O | ILE | A | 231 | 41.137 | 88.088 | 43.536 | 1.00 | 50.68 |
| 1000 | N | ASP | A | 232 | 40.863 | 90.240 | 42.974 | 1.00 | 48.64 |
| 1001 | CA | ASP | A | 232 | 40.086 | 89.859 | 41.797 | 1.00 | 47.47 |
| 1002 | CB | ASP | A | 232 | 38.904 | 90.821 | 41.525 | 1.00 | 46.95 |
| 1003 | CG | ASP | A | 232 | 39.332 | 92.238 | 41.133 | 1.00 | 48.77 |
| 1004 | OD1 | ASP | A | 232 | 40.277 | 92.405 | 40.322 | 1.00 | 48.21 |
| 1005 | OD2 | ASP | A | 232 | 38.683 | 93.194 | 41.624 | 1.00 | 45.17 |
| 1006 | C | ASP | A | 232 | 40.942 | 89.690 | 40.556 | 1.00 | 46.62 |
| 1007 | O | ASP | A | 232 | 42.154 | 89.914 | 40.596 | 1.00 | 46.42 |
| 1008 | N | ASP | A | 233 | 40.304 | 89.265 | 39.467 | 1.00 | 46.49 |
| 1009 | CA | ASP | A | 233 | 40.982 | 89.013 | 38.199 | 1.00 | 48.18 |
| 1010 | CB | ASP | A | 233 | 39.965 | 88.575 | 37.151 | 1.00 | 52.12 |
| 1011 | CG | ASP | A | 233 | 39.694 | 87.069 | 37.177 | 1.00 | 57.52 |
| 1012 | OD1 | ASP | A | 233 | 39.562 | 86.489 | 38.285 | 1.00 | 56.60 |
| 1013 | OD2 | ASP | A | 233 | 39.596 | 86.474 | 36.074 | 1.00 | 59.38 |
| 1014 | C | ASP | A | 233 | 41.777 | 90.190 | 37.668 | 1.00 | 47.78 |
| 1015 | O | ASP | A | 233 | 42.889 | 90.016 | 37.175 | 1.00 | 47.91 |
| 1016 | N | ASN | A | 234 | 41.211 | 91.389 | 37.764 | 1.00 | 45.75 |
| 1017 | CA | ASN | A | 234 | 41.891 | 92.580 | 37.281 | 1.00 | 45.96 |
| 1018 | CB | ASN | A | 234 | 40.943 | 93.765 | 37.316 | 1.00 | 45.13 |
| 1019 | CG | ASN | A | 234 | 39.780 | 93.581 | 36.396 | 1.00 | 46.35 |
| 1020 | OD1 | ASN | A | 234 | 39.960 | 93.329 | 35.203 | 1.00 | 49.07 |
| 1021 | ND2 | ASN | A | 234 | 38.571 | 93.699 | 36.932 | 1.00 | 43.71 |
| 1022 | C | ASN | A | 234 | 43.143 | 92.912 | 38.066 | 1.00 | 46.24 |
| 1023 | O | ASN | A | 234 | 44.174 | 93.234 | 37.488 | 1.00 | 46.25 |
| 1024 | N | GLU | A | 235 | 43.049 | 92.834 | 39.388 | 1.00 | 47.76 |
| 1025 | CA | GLU | A | 235 | 44.182 | 93.128 | 40.253 | 1.00 | 48.46 |
| 1026 | CB | GLU | A | 235 | 43.724 | 93.106 | 41.711 | 1.00 | 49.43 |
| 1027 | CG | GLU | A | 235 | 42.606 | 94.125 | 41.978 | 1.00 | 50.73 |
| 1028 | CD | GLU | A | 235 | 41.942 | 93.956 | 43.338 | 1.00 | 53.14 |
| 1029 | OE1 | GLU | A | 235 | 41.787 | 92.805 | 43.807 | 1.00 | 52.31 |
| 1030 | OE2 | GLU | A | 235 | 41.551 | 94.979 | 43.934 | 1.00 | 55.18 |
| 1031 | C | GLU | A | 235 | 45.289 | 92.111 | 39.992 | 1.00 | 48.60 |
| 1032 | O | GLU | A | 235 | 46.459 | 92.474 | 39.843 | 1.00 | 48.72 |
| 1033 | N | TYR | A | 236 | 44.912 | 90.839 | 39.903 | 1.00 | 48.87 |
| 1034 | CA | TYR | A | 236 | 45.870 | 89.770 | 39.630 | 1.00 | 48.41 |
| 1035 | CB | TYR | A | 236 | 45.154 | 88.422 | 39.678 | 1.00 | 49.95 |
| 1036 | CG | TYR | A | 236 | 45.920 | 87.283 | 39.051 | 1.00 | 53.44 |
| 1037 | CD1 | TYR | A | 236 | 45.651 | 86.886 | 37.747 | 1.00 | 55.68 |
| 1038 | CE1 | TYR | A | 236 | 46.344 | 85.841 | 37.155 | 1.00 | 57.29 |
| 1039 | CD2 | TYR | A | 236 | 46.901 | 86.592 | 39.760 | 1.00 | 52.47 |
| 1040 | CE2 | TYR | A | 236 | 47.606 | 85.542 | 39.172 | 1.00 | 53.08 |
| 1041 | CZ | TYR | A | 236 | 47.315 | 85.167 | 37.870 | 1.00 | 56.41 |
| 1042 | OH | TYR | A | 236 | 47.994 | 84.134 | 37.252 | 1.00 | 56.73 |
| 1043 | C | TYR | A | 236 | 46.577 | 89.962 | 38.282 | 1.00 | 48.08 |
| 1044 | O | TYR | A | 236 | 47.791 | 89.781 | 38.175 | 1.00 | 48.08 |
| 1045 | N | ALA | A | 237 | 45.823 | 90.338 | 37.254 | 1.00 | 48.09 |
| 1046 | CA | ALA | A | 237 | 46.412 | 90.556 | 35.939 | 1.00 | 46.29 |
| 1047 | CB | ALA | A | 237 | 45.337 | 90.916 | 34.935 | 1.00 | 46.10 |
| 1048 | C | ALA | A | 237 | 47.452 | 91.668 | 36.022 | 1.00 | 46.46 |
| 1049 | O | ALA | A | 237 | 48.490 | 91.599 | 35.376 | 1.00 | 45.66 |
| 1050 | N | CYS | A | 238 | 47.170 | 92.699 | 36.816 | 1.00 | 46.82 |
| 1051 | CA | CYS | A | 238 | 48.115 | 93.802 | 36.976 | 1.00 | 48.15 |
| 1052 | CB | CYS | A | 238 | 47.458 | 94.968 | 37.732 | 1.00 | 49.33 |
| 1053 | SG | CYS | A | 238 | 46.241 | 95.936 | 36.761 | 1.00 | 51.88 |
| 1054 | C | CYS | A | 238 | 49.411 | 93.348 | 37.686 | 1.00 | 47.77 |
| 1055 | O | CYS | A | 238 | 50.506 | 93.701 | 37.245 | 1.00 | 47.67 |
| 1056 | N | LEU | A | 239 | 49.303 | 92.568 | 38.766 | 1.00 | 46.50 |
| 1057 | CA | LEU | A | 239 | 50.503 | 92.073 | 39.459 | 1.00 | 48.00 |
| 1058 | CB | LEU | A | 239 | 50.146 | 91.171 | 40.635 | 1.00 | 46.75 |
| 1059 | CG | LEU | A | 239 | 49.706 | 91.835 | 41.927 | 1.00 | 48.33 |
| 1060 | CD1 | LEU | A | 239 | 49.595 | 90.778 | 43.008 | 1.00 | 47.55 |
| 1061 | CD2 | LEU | A | 239 | 50.721 | 92.893 | 42.324 | 1.00 | 49.61 |
| 1062 | C | LEU | A | 239 | 51.333 | 91.245 | 38.497 | 1.00 | 49.12 |
| 1063 | O | LEU | A | 239 | 52.558 | 91.302 | 38.477 | 1.00 | 49.41 |
| 1064 | N | LYS | A | 240 | 50.633 | 90.448 | 37.710 | 1.00 | 50.71 |
| 1065 | CA | LYS | A | 240 | 51.252 | 89.589 | 36.728 | 1.00 | 50.49 |
| 1066 | CB | LYS | A | 240 | 50.126 | 88.828 | 36.053 | 1.00 | 54.00 |
| 1067 | CG | LYS | A | 240 | 50.485 | 87.682 | 35.157 | 1.00 | 57.50 |
| 1068 | CD | LYS | A | 240 | 49.187 | 86.963 | 34.799 | 1.00 | 56.35 |
| 1069 | CE | LYS | A | 240 | 49.320 | 86.109 | 33.558 | 1.00 | 59.43 |
| 1070 | NZ | LYS | A | 240 | 48.042 | 85.379 | 33.300 | 1.00 | 59.29 |
| 1071 | C | LYS | A | 240 | 52.062 | 90.443 | 35.739 | 1.00 | 50.77 |
| 1072 | O | LYS | A | 240 | 53.230 | 90.156 | 35.473 | 1.00 | 52.17 |
| 1073 | N | ALA | A | 241 | 51.457 | 91.516 | 35.225 | 1.00 | 49.05 |
| 1074 | CA | ALA | A | 241 | 52.139 | 92.392 | 34.264 | 1.00 | 47.99 |
| 1075 | CB | ALA | A | 241 | 51.125 | 93.306 | 33.569 | 1.00 | 45.58 |
| 1076 | C | ALA | A | 241 | 53.254 | 93.235 | 34.892 | 1.00 | 48.74 |
| 1077 | O | ALA | A | 241 | 54.284 | 93.473 | 34.257 | 1.00 | 47.88 |
| 1078 | N | ILE | A | 242 | 53.042 | 93.704 | 36.121 | 1.00 | 49.21 |
| 1079 | CA | ILE | A | 242 | 54.052 | 94.502 | 36.818 | 1.00 | 48.58 |
| 1080 | CB | ILE | A | 242 | 53.546 | 94.949 | 38.211 | 1.00 | 46.80 |
| 1081 | CG2 | ILE | A | 242 | 54.710 | 95.441 | 39.065 | 1.00 | 44.00 |
| 1082 | CG1 | ILE | A | 242 | 52.483 | 96.042 | 38.048 | 1.00 | 46.90 |
| 1083 | CD1 | ILE | A | 242 | 51.708 | 96.362 | 39.343 | 1.00 | 44.31 |
| 1084 | C | ILE | A | 242 | 55.330 | 93.663 | 36.985 | 1.00 | 49.40 |
| 1085 | O | ILE | A | 242 | 56.447 | 94.154 | 36.818 | 1.00 | 50.74 |
| 1086 | N | VAL | A | 243 | 55.155 | 92.390 | 37.301 | 1.00 | 48.19 |
| 1087 | CA | VAL | A | 243 | 56.279 | 91.485 | 37.478 | 1.00 | 48.24 |
| 1088 | CB | VAL | A | 243 | 55.783 | 90.154 | 38.104 | 1.00 | 45.34 |
| 1089 | CG1 | VAL | A | 243 | 56.826 | 89.096 | 38.006 | 1.00 | 38.32 |
| 1090 | CG2 | VAL | A | 243 | 55.409 | 90.390 | 39.558 | 1.00 | 45.18 |
| 1091 | C | VAL | A | 243 | 56.984 | 91.218 | 36.142 | 1.00 | 51.49 |
| 1092 | O | VAL | A | 243 | 58.213 | 91.145 | 36.082 | 1.00 | 52.53 |
| 1093 | N | PHE | A | 244 | 56.201 | 91.095 | 35.073 | 1.00 | 54.83 |
| 1094 | CA | PHE | A | 244 | 56.728 | 90.814 | 33.732 | 1.00 | 55.59 |
| 1095 | CB | PHE | A | 244 | 55.575 | 90.453 | 32.796 | 1.00 | 54.57 |
| 1096 | CG | PHE | A | 244 | 56.013 | 89.938 | 31.460 | 1.00 | 53.37 |
| 1097 | CD1 | PHE | A | 244 | 56.555 | 88.670 | 31.337 | 1.00 | 52.35 |
| 1098 | CD2 | PHE | A | 244 | 55.864 | 90.718 | 30.319 | 1.00 | 55.39 |
| 1099 | CE1 | PHE | A | 244 | 56.943 | 88.175 | 30.102 | 1.00 | 53.54 |
| 1100 | CE2 | PHE | A | 244 | 56.248 | 90.234 | 29.069 | 1.00 | 56.32 |
| 1101 | CZ | PHE | A | 244 | 56.789 | 88.959 | 28.959 | 1.00 | 56.06 |
| 1102 | C | PHE | A | 244 | 57.514 | 91.979 | 33.133 | 1.00 | 57.68 |
| 1103 | O | PHE | A | 244 | 58.623 | 91.799 | 32.623 | 1.00 | 58.70 |
| 1104 | N | PHE | A | 245 | 56.935 | 93.172 | 33.187 | 1.00 | 58.41 |
| 1105 | CA | PHE | A | 245 | 57.591 | 94.347 | 32.635 | 1.00 | 59.59 |
| 1106 | CB | PHE | A | 245 | 56.538 | 95.388 | 32.225 | 1.00 | 58.77 |
| 1107 | CG | PHE | A | 245 | 55.705 | 94.962 | 31.037 | 1.00 | 58.78 |
| 1108 | CD1 | PHE | A | 245 | 56.298 | 94.780 | 29.787 | 1.00 | 58.21 |
| 1109 | CD2 | PHE | A | 245 | 54.343 | 94.684 | 31.176 | 1.00 | 58.58 |
| 1110 | CE1 | PHE | A | 245 | 55.550 | 94.325 | 28.692 | 1.00 | 57.32 |
| 1111 | CE2 | PHE | A | 245 | 53.585 | 94.225 | 30.082 | 1.00 | 57.14 |
| 1112 | CZ | PHE | A | 245 | 54.193 | 94.046 | 28.840 | 1.00 | 56.15 |
| 1113 | C | PHE | A | 245 | 58.600 | 94.931 | 33.614 | 1.00 | 61.09 |
| 1114 | O | PHE | A | 245 | 58.423 | 96.030 | 34.132 | 1.00 | 60.20 |
| 1115 | N | ASP | A | 246 | 59.668 | 94.172 | 33.854 | 1.00 | 63.54 |
| 1116 | CA | ASP | A | 246 | 60.731 | 94.582 | 34.762 | 1.00 | 65.15 |
| 1117 | CB | ASP | A | 246 | 61.227 | 93.397 | 35.578 | 1.00 | 64.55 |
| 1118 | CG | ASP | A | 246 | 61.961 | 93.827 | 36.837 | 1.00 | 67.76 |
| 1119 | OD1 | ASP | A | 246 | 62.644 | 94.880 | 36.805 | 1.00 | 65.16 |
| 1120 | OD2 | ASP | A | 246 | 61.857 | 93.109 | 37.860 | 1.00 | 69.69 |
| 1121 | C | ASP | A | 246 | 61.911 | 95.159 | 33.993 | 1.00 | 66.87 |
| 1122 | O | ASP | A | 246 | 62.597 | 94.438 | 33.268 | 1.00 | 66.84 |
| 1123 | N | PRO | A | 247 | 62.177 | 96.466 | 34.157 | 1.00 | 68.77 |
| 1124 | CD | PRO | A | 247 | 61.425 | 97.421 | 34.991 | 1.00 | 67.95 |
| 1125 | CA | PRO | A | 247 | 63.285 | 97.135 | 33.469 | 1.00 | 70.71 |
| 1126 | CB | PRO | A | 247 | 63.047 | 98.611 | 33.789 | 1.00 | 67.96 |
| 1127 | CG | PRO | A | 247 | 62.398 | 98.564 | 35.121 | 1.00 | 66.76 |
| 1128 | C | PRO | A | 247 | 64.670 | 96.652 | 33.898 | 1.00 | 74.75 |
| 1129 | O | PRO | A | 247 | 65.679 | 96.999 | 33.277 | 1.00 | 76.67 |
| 1130 | N | ASP | A | 248 | 64.716 | 95.833 | 34.948 | 1.00 | 77.65 |
| 1131 | CA | ASP | A | 248 | 65.979 | 95.309 | 35.464 | 1.00 | 79.51 |
| 1132 | CB | ASP | A | 248 | 65.835 | 94.974 | 36.953 | 1.00 | 83.13 |
| 1133 | CG | ASP | A | 248 | 65.384 | 93.526 | 37.197 | 1.00 | 86.52 |
| 1134 | OD1 | ASP | A | 248 | 64.692 | 92.949 | 36.325 | 1.00 | 88.52 |
| 1135 | OD2 | ASP | A | 248 | 65.710 | 92.966 | 38.271 | 1.00 | 87.60 |
| 1136 | C | ASP | A | 248 | 66.436 | 94.055 | 34.718 | 1.00 | 79.83 |
| 1137 | O | ASP | A | 248 | 67.458 | 93.466 | 35.057 | 1.00 | 79.98 |
| 1138 | N | ALA | A | 249 | 65.683 | 93.647 | 33.705 | 1.00 | 80.13 |
| 1139 | CA | ALA | A | 249 | 66.023 | 92.445 | 32.955 | 1.00 | 81.61 |
| 1140 | CB | ALA | A | 249 | 64.879 | 92.080 | 32.037 | 1.00 | 81.13 |
| 1141 | C | ALA | A | 249 | 67.338 | 92.471 | 32.168 | 1.00 | 83.45 |
| 1142 | O | ALA | A | 249 | 67.683 | 93.452 | 31.497 | 1.00 | 83.67 |
| 1143 | N | LYS | A | 250 | 68.038 | 91.345 | 32.263 | 1.00 | 85.37 |
| 1144 | CA | LYS | A | 250 | 69.329 | 91.074 | 31.632 | 1.00 | 86.69 |
| 1145 | CB | LYS | A | 250 | 69.781 | 89.672 | 32.069 | 1.00 | 89.66 |
| 1146 | CG | LYS | A | 250 | 71.047 | 89.105 | 31.432 | 1.00 | 92.26 |
| 1147 | CD | LYS | A | 250 | 71.188 | 87.630 | 31.834 | 1.00 | 94.47 |
| 1148 | CE | LYS | A | 250 | 72.461 | 86.972 | 31.300 | 1.00 | 95.75 |
| 1149 | NZ | LYS | A | 250 | 72.507 | 85.516 | 31.655 | 1.00 | 95.95 |
| 1150 | C | LYS | A | 250 | 69.332 | 91.163 | 30.104 | 1.00 | 86.09 |
| 1151 | O | LYS | A | 250 | 68.820 | 90.275 | 29.419 | 1.00 | 84.97 |
| 1152 | N | GLY | A | 251 | 69.914 | 92.237 | 29.578 | 1.00 | 85.72 |
| 1153 | CA | GLY | A | 251 | 70.001 | 92.387 | 28.137 | 1.00 | 84.58 |
| 1154 | C | GLY | A | 251 | 68.959 | 93.239 | 27.440 | 1.00 | 83.64 |
| 1155 | O | GLY | A | 251 | 68.885 | 93.227 | 26.210 | 1.00 | 83.59 |
| 1156 | N | LEU | A | 252 | 68.146 | 93.968 | 28.194 | 1.00 | 82.40 |
| 1157 | CA | LEU | A | 252 | 67.148 | 94.812 | 27.556 | 1.00 | 81.56 |
| 1158 | CB | LEU | A | 252 | 66.201 | 95.415 | 28.599 | 1.00 | 79.55 |
| 1159 | CG | LEU | A | 252 | 65.150 | 94.466 | 29.166 | 1.00 | 76.78 |
| 1160 | CD1 | LEU | A | 252 | 64.349 | 95.187 | 30.222 | 1.00 | 76.49 |
| 1161 | CD2 | LEU | A | 252 | 64.237 | 93.979 | 28.048 | 1.00 | 76.00 |
| 1162 | C | LEU | A | 252 | 67.878 | 95.917 | 26.795 | 1.00 | 81.16 |
| 1163 | O | LEU | A | 252 | 68.650 | 96.674 | 27.382 | 1.00 | 79.97 |
| 1164 | N | SER | A | 253 | 67.657 | 95.989 | 25.487 | 1.00 | 81.23 |
| 1165 | CA | SER | A | 253 | 68.306 | 97.012 | 24.685 | 1.00 | 81.61 |
| 1166 | CB | SER | A | 253 | 67.995 | 96.824 | 23.197 | 1.00 | 81.75 |
| 1167 | OG | SER | A | 253 | 66.691 | 96.323 | 22.990 | 1.00 | 82.66 |
| 1168 | C | SER | A | 253 | 67.863 | 98.383 | 25.153 | 1.00 | 82.13 |
| 1169 | O | SER | A | 253 | 68.637 | 99.332 | 25.109 | 1.00 | 82.65 |
| 1170 | N | ASP | A | 254 | 66.621 | 98.492 | 25.614 | 1.00 | 83.11 |
| 1171 | CA | ASP | A | 254 | 66.127 | 99.772 | 26.110 | 1.00 | 82.79 |
| 1172 | CB | ASP | A | 254 | 65.424 | 100.547 | 24.991 | 1.00 | 82.13 |
| 1173 | CG | ASP | A | 254 | 64.827 | 101.859 | 25.479 | 1.00 | 82.66 |
| 1174 | OD1 | ASP | A | 254 | 65.492 | 102.585 | 26.255 | 1.00 | 81.63 |
| 1175 | OD2 | ASP | A | 254 | 63.688 | 102.168 | 25.076 | 1.00 | 82.97 |
| 1176 | C | ASP | A | 254 | 65.200 | 99.626 | 27.317 | 1.00 | 82.18 |
| 1177 | O | ASP | A | 254 | 63.978 | 99.548 | 27.179 | 1.00 | 82.68 |
| 1178 | N | PRO | A | 255 | 65.788 | 99.578 | 28.521 | 1.00 | 81.01 |
| 1179 | CD | PRO | A | 255 | 67.236 | 99.400 | 28.707 | 1.00 | 80.96 |
| 1180 | CA | PRO | A | 255 | 65.089 | 99.444 | 29.803 | 1.00 | 80.76 |
| 1181 | CB | PRO | A | 255 | 66.236 | 99.331 | 30.817 | 1.00 | 81.56 |
| 1182 | CG | PRO | A | 255 | 67.427 | 99.910 | 30.096 | 1.00 | 81.32 |
| 1183 | C | PRO | A | 255 | 64.097 | 100.545 | 30.169 | 1.00 | 79.69 |
| 1184 | O | PRO | A | 255 | 63.122 | 100.288 | 30.865 | 1.00 | 80.91 |
| 1185 | N | VAL | A | 256 | 64.336 | 101.769 | 29.711 | 1.00 | 78.91 |
| 1186 | CA | VAL | A | 256 | 63.429 | 102.869 | 30.030 | 1.00 | 77.27 |
| 1187 | CB | VAL | A | 256 | 63.992 | 104.232 | 29.550 | 1.00 | 78.23 |
| 1188 | CG1 | VAL | A | 256 | 62.983 | 105.341 | 29.832 | 1.00 | 77.91 |
| 1189 | CG2 | VAL | A | 256 | 65.308 | 104.533 | 30.257 | 1.00 | 76.94 |
| 1190 | C | VAL | A | 256 | 62.058 | 102.663 | 29.403 | 1.00 | 75.83 |
| 1191 | O | VAL | A | 256 | 61.032 | 102.881 | 30.044 | 1.00 | 75.15 |
| 1192 | N | LYS | A | 257 | 62.046 | 102.243 | 28.145 | 1.00 | 75.65 |
| 1193 | CA | LYS | A | 257 | 60.796 | 102.002 | 27.434 | 1.00 | 75.92 |
| 1194 | CB | LYS | A | 257 | 61.088 | 101.424 | 26.043 | 1.00 | 79.24 |
| 1195 | CG | LYS | A | 257 | 59.871 | 101.320 | 25.137 | 1.00 | 83.31 |
| 1196 | CD | LYS | A | 257 | 60.245 | 100.876 | 23.722 | 1.00 | 86.30 |
| 1197 | CE | LYS | A | 257 | 59.025 | 100.944 | 22.802 | 1.00 | 88.69 |
| 1198 | NZ | LYS | A | 257 | 59.298 | 100.474 | 21.417 | 1.00 | 89.02 |
| 1199 | C | LYS | A | 257 | 59.941 | 101.024 | 28.236 | 1.00 | 74.13 |
| 1200 | O | LYS | A | 257 | 58.718 | 101.142 | 28.277 | 1.00 | 73.74 |
| 1201 | N | ILE | A | 258 | 60.605 | 100.066 | 28.876 | 1.00 | 72.04 |
| 1202 | CA | ILE | A | 258 | 59.942 | 99.052 | 29.689 | 1.00 | 70.80 |
| 1203 | CB | ILE | A | 258 | 60.887 | 97.861 | 29.955 | 1.00 | 69.63 |
| 1204 | CG2 | ILE | A | 258 | 60.220 | 96.857 | 30.881 | 1.00 | 66.22 |
| 1205 | CG1 | ILE | A | 258 | 61.305 | 97.221 | 28.630 | 1.00 | 68.99 |
| 1206 | CD1 | ILE | A | 258 | 60.186 | 96.543 | 27.878 | 1.00 | 68.59 |
| 1207 | C | ILE | A | 258 | 59.480 | 99.608 | 31.039 | 1.00 | 71.95 |
| 1208 | O | ILE | A | 258 | 58.402 | 99.258 | 31.524 | 1.00 | 71.82 |
| 1209 | N | LYS | A | 259 | 60.302 | 100.465 | 31.646 | 1.00 | 71.81 |
| 1210 | CA | LYS | A | 259 | 59.971 | 101.056 | 32.935 | 1.00 | 71.37 |
| 1211 | CB | LYS | A | 259 | 61.122 | 101.934 | 33.428 | 1.00 | 71.91 |
| 1212 | CG | LYS | A | 259 | 60.824 | 102.721 | 34.696 | 1.00 | 74.08 |
| 1213 | CD | LYS | A | 259 | 62.072 | 103.445 | 35.170 | 1.00 | 77.52 |
| 1214 | CE | LYS | A | 259 | 61.741 | 104.697 | 35.960 | 1.00 | 80.33 |
| 1215 | NZ | LYS | A | 259 | 62.972 | 105.515 | 36.186 | 1.00 | 84.19 |
| 1216 | C | LYS | A | 259 | 58.700 | 101.874 | 32.817 | 1.00 | 70.78 |
| 1217 | O | LYS | A | 259 | 57.927 | 101.974 | 33.764 | 1.00 | 72.63 |
| 1218 | N | ASN | A | 260 | 58.472 | 102.446 | 31.641 | 1.00 | 70.19 |
| 1219 | CA | ASN | A | 260 | 57.273 | 103.244 | 31.415 | 1.00 | 69.79 |
| 1220 | CB | ASN | A | 260 | 57.490 | 104.188 | 30.236 | 1.00 | 71.17 |
| 1221 | CG | ASN | A | 260 | 58.720 | 105.052 | 30.419 | 1.00 | 74.72 |
| 1222 | OD1 | ASN | A | 260 | 58.922 | 105.657 | 31.479 | 1.00 | 74.47 |
| 1223 | ND2 | ASN | A | 260 | 59.556 | 105.115 | 29.390 | 1.00 | 77.15 |
| 1224 | C | ASN | A | 260 | 56.058 | 102.361 | 31.172 | 1.00 | 68.04 |
| 1225 | O | ASN | A | 260 | 54.949 | 102.676 | 31.614 | 1.00 | 67.39 |
| 1226 | N | MET | A | 261 | 56.266 | 101.257 | 30.468 | 1.00 | 65.33 |
| 1227 | CA | MET | A | 261 | 55.180 | 100.333 | 30.209 | 1.00 | 64.61 |
| 1228 | CB | MET | A | 261 | 55.686 | 99.163 | 29.367 | 1.00 | 65.40 |
| 1229 | CG | MET | A | 261 | 56.114 | 99.580 | 27.962 | 1.00 | 69.44 |
| 1230 | SD | MET | A | 261 | 57.072 | 98.352 | 27.012 | 1.00 | 72.90 |
| 1231 | CE | MET | A | 261 | 55.831 | 97.294 | 26.447 | 1.00 | 69.88 |
| 1232 | C | MET | A | 261 | 54.674 | 99.841 | 31.561 | 1.00 | 63.57 |
| 1233 | O | MET | A | 261 | 53.469 | 99.731 | 31.793 | 1.00 | 63.29 |
| 1234 | N | ARG | A | 262 | 55.606 | 99.566 | 32.464 | 1.00 | 62.82 |
| 1235 | CA | ARG | A | 262 | 55.241 | 99.089 | 33.783 | 1.00 | 61.09 |
| 1236 | CB | ARG | A | 262 | 56.466 | 98.585 | 34.546 | 1.00 | 60.78 |
| 1237 | CG | ARG | A | 262 | 56.132 | 98.203 | 35.970 | 1.00 | 62.33 |
| 1238 | CD | ARG | A | 262 | 56.995 | 97.086 | 36.488 | 1.00 | 64.80 |
| 1239 | NE | ARG | A | 262 | 58.042 | 97.559 | 37.384 | 1.00 | 67.91 |
| 1240 | CZ | ARG | A | 262 | 58.792 | 96.759 | 38.139 | 1.00 | 71.63 |
| 1241 | NH1 | ARG | A | 262 | 58.616 | 95.434 | 38.112 | 1.00 | 67.64 |
| 1242 | NH2 | ARG | A | 262 | 59.721 | 97.288 | 38.925 | 1.00 | 74.13 |
| 1243 | C | ARG | A | 262 | 54.552 | 100.174 | 34.584 | 1.00 | 60.74 |
| 1244 | O | ARG | A | 262 | 53.684 | 99.881 | 35.402 | 1.00 | 61.57 |
| 1245 | N | PHE | A | 263 | 54.925 | 101.428 | 34.350 | 1.00 | 60.30 |
| 1246 | CA | PHE | A | 263 | 54.308 | 102.523 | 35.088 | 1.00 | 59.97 |
| 1247 | CB | PHE | A | 263 | 54.998 | 103.853 | 34.783 | 1.00 | 58.85 |
| 1248 | CG | PHE | A | 263 | 54.642 | 104.952 | 35.754 | 1.00 | 59.22 |
| 1249 | CD1 | PHE | A | 263 | 55.188 | 104.970 | 37.039 | 1.00 | 59.56 |
| 1250 | CD2 | PHE | A | 263 | 53.740 | 105.950 | 35.397 | 1.00 | 57.08 |
| 1251 | CE1 | PHE | A | 263 | 54.838 | 105.970 | 37.952 | 1.00 | 59.73 |
| 1252 | CE2 | PHE | A | 263 | 53.383 | 106.952 | 36.298 | 1.00 | 57.13 |
| 1253 | CZ | PHE | A | 263 | 53.931 | 106.966 | 37.576 | 1.00 | 59.03 |
| 1254 | C | PHE | A | 263 | 52.817 | 102.631 | 34.769 | 1.00 | 59.75 |
| 1255 | O | PHE | A | 263 | 52.004 | 102.908 | 35.655 | 1.00 | 59.30 |
| 1256 | N | GLN | A | 264 | 52.462 | 102.409 | 33.506 | 1.00 | 60.29 |
| 1257 | CA | GLN | A | 264 | 51.062 | 102.468 | 33.087 | 1.00 | 61.03 |
| 1258 | CG | GLN | A | 264 | 50.933 | 102.224 | 31.581 | 1.00 | 64.07 |
| 1259 | CG | GLN | A | 264 | 51.512 | 103.315 | 30.681 | 1.00 | 70.10 |
| 1260 | CD | GLN | A | 264 | 51.228 | 103.055 | 29.194 | 1.00 | 74.41 |
| 1261 | OE1 | GLN | A | 264 | 50.095 | 103.229 | 28.718 | 1.00 | 77.09 |
| 1262 | NE2 | GLN | A | 264 | 52.257 | 102.624 | 28.457 | 1.00 | 74.80 |
| 1263 | C | GLN | A | 264 | 50.234 | 101.425 | 33.836 | 1.00 | 59.01 |
| 1264 | O | GLN | A | 264 | 49.139 | 101.722 | 34.323 | 1.00 | 58.30 |
| 1265 | N | VAL | A | 265 | 50.758 | 100.205 | 33.920 | 1.00 | 56.49 |
| 1266 | CA | VAL | A | 265 | 50.072 | 99.125 | 34.619 | 1.00 | 55.96 |
| 1267 | CG | VAL | A | 265 | 50.846 | 97.795 | 34.478 | 1.00 | 55.22 |
| 1268 | CG1 | VAL | A | 265 | 50.087 | 96.663 | 35.161 | 1.00 | 54.32 |
| 1269 | CG2 | VAL | A | 265 | 51.068 | 97.482 | 33.008 | 1.00 | 53.47 |
| 1270 | C | VAL | A | 265 | 49.968 | 99.499 | 36.097 | 1.00 | 55.96 |
| 1271 | O | VAL | A | 265 | 48.944 | 99.279 | 36.751 | 1.00 | 53.81 |
| 1272 | N | GLN | A | 266 | 51.047 | 100.080 | 36.605 | 1.00 | 57.55 |
| 1273 | CA | GLN | A | 266 | 51.141 | 100.528 | 37.990 | 1.00 | 59.27 |
| 1274 | CG | GLN | A | 266 | 52.459 | 101.251 | 38.171 | 1.00 | 62.94 |
| 1275 | CG | GLN | A | 266 | 53.283 | 100.820 | 39.343 | 1.00 | 68.25 |
| 1276 | CD | GLN | A | 266 | 54.415 | 101.793 | 39.579 | 1.00 | 71.68 |
| 1277 | OE1 | GLN | A | 266 | 54.258 | 102.784 | 40.310 | 1.00 | 72.80 |
| 1278 | NE2 | GLN | A | 266 | 55.559 | 101.539 | 38.936 | 1.00 | 69.00 |
| 1279 | C | GLN | A | 266 | 50.001 | 101.502 | 38.305 | 1.00 | 59.03 |
| 1280 | O | GLN | A | 266 | 49.240 | 101.319 | 39.267 | 1.00 | 58.11 |
| 1281 | N | ILE | A | 267 | 49.920 | 102.555 | 37.494 | 1.00 | 57.81 |
| 1282 | CA | ILE | A | 267 | 48.884 | 103.580 | 37.626 | 1.00 | 55.82 |
| 1283 | CG | ILE | A | 267 | 49.075 | 104.704 | 36.569 | 1.00 | 53.53 |
| 1284 | CG2 | ILE | A | 267 | 47.859 | 105.604 | 36.531 | 1.00 | 53.33 |
| 1285 | CG1 | ILE | A | 267 | 50.312 | 105.538 | 36.906 | 1.00 | 53.35 |
| 1286 | CD1 | ILE | A | 267 | 50.162 | 106.379 | 38.167 | 1.00 | 50.08 |
| 1287 | C | ILE | A | 267 | 47.516 | 102.928 | 37.435 | 1.00 | 55.66 |
| 1288 | O | ILE | A | 267 | 46.602 | 103.132 | 38.245 | 1.00 | 56.39 |
| 1289 | N | GLY | A | 268 | 47.387 | 102.145 | 36.360 | 1.00 | 53.70 |
| 1290 | CA | GLY | A | 268 | 46.145 | 101.450 | 36.085 | 1.00 | 51.40 |
| 1291 | C | GLY | A | 268 | 45.630 | 100.727 | 37.319 | 1.00 | 49.74 |
| 1292 | O | GLY | A | 268 | 44.478 | 100.895 | 37.711 | 1.00 | 49.92 |
| 1293 | N | LEU | A | 269 | 46.488 | 99.933 | 37.946 | 1.00 | 48.32 |
| 1294 | CA | LEU | A | 269 | 46.096 | 99.188 | 39.133 | 1.00 | 48.09 |
| 1295 | CB | LEU | A | 269 | 47.249 | 98.302 | 39.605 | 1.00 | 45.26 |
| 1296 | CG | LEU | A | 269 | 46.975 | 97.534 | 40.902 | 1.00 | 44.87 |
| 1297 | CD1 | LEU | A | 269 | 45.757 | 96.639 | 40.733 | 1.00 | 45.06 |
| 1298 | CD2 | LEU | A | 269 | 48.188 | 96.715 | 41.280 | 1.00 | 43.07 |
| 1299 | C | LEU | A | 269 | 45.629 | 100.077 | 40.288 | 1.00 | 49.72 |
| 1300 | O | LEU | A | 269 | 44.596 | 99.815 | 40.909 | 1.00 | 49.03 |
| 1301 | N | GLU | A | 270 | 46.383 | 101.127 | 40.586 | 1.00 | 53.23 |
| 1302 | CA | GLU | A | 270 | 46.005 | 102.005 | 41.687 | 1.00 | 55.47 |
| 1303 | CB | GLU | A | 270 | 47.109 | 103.020 | 41.975 | 1.00 | 56.29 |
| 1304 | CG | GLU | A | 270 | 46.764 | 103.939 | 43.120 | 1.00 | 59.90 |
| 1305 | CD | GLU | A | 270 | 47.932 | 104.789 | 43.581 | 1.00 | 61.37 |
| 1306 | OE1 | GLU | A | 270 | 48.821 | 105.088 | 42.756 | 1.00 | 61.19 |
| 1307 | OE2 | GLU | A | 270 | 47.946 | 105.171 | 44.774 | 1.00 | 64.89 |
| 1308 | C | GLU | A | 270 | 44.688 | 102.713 | 41.384 | 1.00 | 56.76 |
| 1309 | O | GLU | A | 270 | 43.803 | 102.786 | 42.252 | 1.00 | 56.84 |
| 1310 | N | ASP | A | 271 | 44.546 | 103.229 | 40.164 | 1.00 | 55.55 |
| 1311 | CA | ASP | A | 271 | 43.300 | 103.885 | 39.809 | 1.00 | 56.54 |
| 1312 | CB | ASP | A | 271 | 43.313 | 104.357 | 38.352 | 1.00 | 57.29 |
| 1313 | CG | ASP | A | 271 | v.189 | 105.586 | 38.130 | 1.00 | 58.73 |
| 1314 | OD1 | ASP | A | 271 | 44.418 | 106.366 | 39.088 | 1.00 | 57.46 |
| 1315 | OD2 | ASP | A | 271 | 44.633 | 105.778 | 36.974 | 1.00 | 59.16 |
| 1316 | C | ASP | A | 271 | 42.151 | 102.890 | 40.017 | 1.00 | 58.03 |
| 1317 | O | ASP | A | 271 | 41.209 | 103.168 | 40.763 | 1.00 | 59.82 |
| 1318 | N | TYR | A | 272 | 42.245 | 101.725 | 39.377 | 1.00 | 57.20 |
| 1319 | CA | TYR | A | 272 | 41.213 | 100.696 | 39.482 | 1.00 | 55.43 |
| 1320 | CB | TYR | A | 272 | 41.687 | 99.400 | 38.784 | 1.00 | 53.36 |
| 1321 | CG | TYR | A | 272 | 40.780 | 98.182 | 38.971 | 1.00 | 51.32 |
| 1322 | CD1 | TYR | A | 272 | 41.003 | 97.264 | 40.012 | 1.00 | 48.86 |
| 1323 | CE1 | TYR | A | 272 | 40.155 | 96.165 | 40.217 | 1.00 | 47.73 |
| 1324 | CD2 | TYR | A | 272 | 39.679 | 97.968 | 38.134 | 1.00 | 49.11 |
| 1325 | CE2 | TYR | A | 272 | 38.824 | 96.877 | 38.327 | 1.00 | 48.47 |
| 1326 | CZ | TYR | A | 272 | 39.065 | 95.982 | 39.371 | 1.00 | 49.81 |
| 1327 | OH | TYR | A | 272 | 38.216 | 94.913 | 39.568 | 1.00 | 48.08 |
| 1328 | C | TYR | A | 272 | 40.813 | 100.425 | 40.933 | 1.00 | 57.13 |
| 1329 | O | TYR | A | 272 | 39.630 | 100.323 | 41.247 | 1.00 | 55.46 |
| 1330 | N | ILE | A | 273 | 41.796 | 100.325 | 41.823 | 1.00 | 60.05 |
| 1331 | CA | ILE | A | 273 | 41.501 | 100.061 | 43.227 | 1.00 | 61.62 |
| 1332 | CB | ILE | A | 273 | 42.785 | 99.830 | 44.057 | 1.00 | 61.50 |
| 1333 | CG2 | ILE | A | 273 | 42.435 | 99.656 | 45.527 | 1.00 | 60.45 |
| 1334 | CG1 | ILE | A | 273 | 43.506 | 98.574 | 43.574 | 1.00 | 62.84 |
| 1335 | CD1 | ILE | A | 273 | 44.812 | 98.314 | 44.310 | 1.00 | 62.28 |
| 1336 | C | ILE | A | 273 | 40.722 | 101.190 | 43.882 | 1.00 | 62.81 |
| 1337 | O | ILE | A | 273 | 39.798 | 100.937 | 44.648 | 1.00 | 63.09 |
| 1338 | N | ASN | A | 274 | 41.078 | 102.433 | 43.586 | 1.00 | 64.16 |
| 1339 | CA | ASN | A | 274 | 40.380 | 103.545 | 44.218 | 1.00 | 67.47 |
| 1340 | CB | ASN | A | 274 | 41.128 | 104.861 | 43.983 | 1.00 | 65.58 |
| 1341 | CG | ASN | A | 274 | 42.460 | 104.918 | 44.726 | 1.00 | 64.81 |
| 1342 | OD1 | ASN | A | 274 | 42.589 | 104.407 | 45.846 | 1.00 | 63.28 |
| 1343 | ND2 | ASN | A | 274 | 43.450 | 105.556 | 44.113 | 1.00 | 63.86 |
| 1344 | C | ASN | A | 274 | 38.902 | 103.701 | 43.860 | 1.00 | 70.39 |
| 1345 | O | ASN | A | 274 | 38.155 | 104.333 | 44.607 | 1.00 | 70.27 |
| 1346 | N | ASP | A | 275 | 38.476 | 103.120 | 42.738 | 1.00 | 74.20 |
| 1347 | CA | ASP | A | 275 | 37.068 | 103.179 | 42.316 | 1.00 | 77.09 |
| 1348 | CB | ASP | A | 275 | 36.889 | 102.586 | 40.914 | 1.00 | 77.74 |
| 1349 | CG | ASP | A | 275 | 37.568 | 103.392 | 39.827 | 1.00 | 79.45 |
| 1350 | OD1 | ASP | A | 275 | 37.578 | 104.633 | 39.930 | 1.00 | 80.80 |
| 1351 | OD2 | ASP | A | 275 | 38.069 | 102.787 | 38.851 | 1.00 | 79.73 |
| 1352 | C | ASP | A | 275 | 36.207 | 102.333 | 43.261 | 1.00 | 80.11 |
| 1353 | O | ASP | A | 275 | 35.038 | 102.097 | 43.001 | 1.00 | 81.46 |
| 1354 | N | ARG | A | 276 | 36.789 | 101.912 | 44.371 | 1.00 | 83.09 |
| 1355 | CA | ARG | A | 276 | 36.149 | 101.012 | 45.321 | 1.00 | 86.28 |
| 1356 | CB | ARG | A | 276 | 37.199 | 99.960 | 45.606 | 1.00 | 86.11 |
| 1357 | CG | ARG | A | 276 | 36.773 | 98.630 | 46.075 | 1.00 | 85.26 |
| 1358 | CD | ARG | A | 276 | 38.053 | 97.830 | 46.220 | 1.00 | 82.02 |
| 1359 | NE | ARG | A | 276 | 38.049 | 97.031 | 47.431 | 1.00 | 80.67 |
| 1360 | CZ | ARG | A | 276 | 37.729 | 95.747 | 47.463 | 1.00 | 81.52 |
| 1361 | NH1 | ARG | A | 276 | 37.398 | 95.129 | 46.340 | 1.00 | 83.68 |
| 1362 | NH2 | ARG | A | 276 | 37.734 | 95.081 | 48.609 | 1.00 | 81.37 |
| 1363 | C | ARG | A | 276 | 35.637 | 101.613 | 46.646 | 1.00 | 90.21 |
| 1364 | O | ARG | A | 276 | 36.183 | 102.610 | 47.132 | 1.00 | 91.64 |
| 1365 | N | GLN | A | 277 | 34.603 | 100.994 | 47.229 | 1.00 | 93.27 |
| 1366 | CA | GLN | A | 277 | 34.048 | 101.441 | 48.521 | 1.00 | 94.84 |
| 1367 | CB | GLN | A | 277 | 32.518 | 101.440 | 48.513 | 1.00 | 95.71 |
| 1368 | CG | GLN | A | 277 | 31.886 | 100.885 | 47.262 | 1.00 | 95.87 |
| 1369 | CD | GLN | A | 277 | 30.567 | 100.203 | 47.557 | 1.00 | 95.95 |
| 1370 | OE1 | GLN | A | 277 | 29.933 | 99.648 | 46.663 | 1.00 | 95.95 |
| 1371 | NE2 | GLN | A | 277 | 30.150 | 100.232 | 48.824 | 1.00 | 95.95 |
| 1372 | C | GLN | A | 277 | 34.568 | 100.555 | 49.671 | 1.00 | 95.30 |
| 1373 | O | GLN | A | 277 | 33.853 | 99.756 | 50.284 | 1.00 | 94.08 |
| 1374 | N | TYR | A | 278 | 35.860 | 100.755 | 49.893 | 1.00 | 95.95 |
| 1375 | CA | TYR | A | 278 | 36.742 | 100.143 | 50.880 | 1.00 | 95.95 |
| 1376 | CB | TYR | A | 278 | 36.084 | 99.872 | 52.239 | 1.00 | 95.95 |
| 1377 | CG | TYR | A | 278 | 36.872 | 100.634 | 53.307 | 1.00 | 95.95 |
| 1378 | CD1 | TYR | A | 278 | 36.560 | 101.966 | 53.613 | 1.00 | 95.95 |
| 1379 | CE1 | TYR | A | 278 | 37.383 | 102.736 | 54.450 | 1.00 | 95.95 |
| 1380 | CD2 | TYR | A | 278 | 38.037 | 100.086 | 53.877 | 1.00 | 95.95 |
| 1381 | CE2 | TYR | A | 278 | 38.865 | 100.850 | 54.713 | 1.00 | 95.95 |
| 1382 | CZ | TYR | A | 278 | 38.531 | 102.175 | 54.990 | 1.00 | 95.95 |
| 1383 | OH | TYR | A | 278 | 39.350 | 102.952 | 55.783 | 1.00 | 95.95 |
| 1384 | C | TYR | A | 278 | 37.653 | 98.997 | 50.484 | 1.00 | 95.95 |
| 1385 | O | TYR | A | 278 | 37.482 | 98.330 | 49.457 | 1.00 | 95.95 |
| 1386 | N | ASP | A | 279 | 38.616 | 98.801 | 51.380 | 1.00 | 95.73 |
| 1387 | CA | ASP | A | 279 | 39.777 | 97.942 | 51.242 | 1.00 | 95.32 |
| 1388 | CB | ASP | A | 279 | 39.602 | 96.502 | 50.779 | 1.00 | 95.95 |
| 1389 | CG | ASP | A | 279 | 40.972 | 95.795 | 50.617 | 1.00 | 95.95 |
| 1390 | OD1 | ASP | A | 279 | 41.982 | 96.501 | 50.378 | 1.00 | 95.95 |
| 1391 | OD2 | ASP | A | 279 | 41.062 | 94.560 | 50.730 | 1.00 | 95.95 |
| 1392 | C | ASP | A | 279 | 40.318 | 98.726 | 50.070 | 1.00 | 94.82 |
| 1393 | O | ASP | A | 279 | 40.751 | 98.186 | 49.056 | 1.00 | 95.95 |
| 1394 | N | SER | A | 280 | 40.164 | 100.026 | 50.201 | 1.00 | 91.60 |
| 1395 | CA | SER | A | 280 | 40.665 | 100.929 | 49.210 | 1.00 | 87.92 |
| 1396 | CB | SER | A | 280 | 39.609 | 101.974 | 48.882 | 1.00 | 87.31 |
| 1397 | OG | SER | A | 280 | 39.990 | 102.694 | 47.730 | 1.00 | 87.85 |
| 1398 | C | SER | A | 280 | 41.799 | 101.516 | 50.026 | 1.00 | 86.39 |
| 1399 | O | SER | A | 280 | 42.824 | 101.934 | 49.493 | 1.00 | 87.98 |
| 1400 | N | ARG | A | 281 | 41.610 | 101.492 | 51.343 | 1.00 | 83.31 |
| 1401 | CA | ARG | A | 281 | 42.584 | 102.015 | 52.278 | 1.00 | 80.17 |
| 1402 | CG | ARG | A | 281 | 41.892 | 102.434 | 53.588 | 1.00 | 81.27 |
| 1403 | CG | ARG | A | 281 | 42.763 | 103.216 | 54.584 | 1.00 | 84.29 |
| 1404 | CD | ARG | A | 281 | 42.521 | 104.729 | 54.557 | 1.00 | 86.17 |
| 1405 | NE | ARG | A | 281 | 43.256 | 105.407 | 55.629 | 1.00 | 88.14 |
| 1406 | CZ | ARG | A | 281 | 43.245 | 105.022 | 56.905 | 1.00 | 89.51 |
| 1407 | NH1 | ARG | A | 281 | 42.542 | 103.959 | 57.288 | 1.00 | 89.09 |
| 1408 | NH2 | ARG | A | 281 | 43.941 | 105.705 | 57.808 | 1.00 | 89.59 |
| 1409 | C | ARG | A | 281 | 43.608 | 100.920 | 52.531 | 1.00 | 77.05 |
| 1410 | O | ARG | A | 281 | 43.334 | 99.936 | 53.224 | 1.00 | 77.26 |
| 1411 | N | GLY | A | 282 | 44.784 | 101.084 | 51.932 | 1.00 | 72.58 |
| 1412 | CA | GLY | A | 282 | 45.856 | 100.134 | 52.116 | 1.00 | 67.41 |
| 1413 | C | GLY | A | 282 | 45.928 | 98.955 | 51.168 | 1.00 | 64.88 |
| 1414 | O | GLY | A | 282 | 46.907 | 98.217 | 51.208 | 1.00 | 65.99 |
| 1415 | N | ARG | A | 283 | 44.917 | 98.780 | 50.321 | 1.00 | 60.78 |
| 1416 | CA | ARG | A | 283 | 44.864 | 97.668 | 49.374 | 1.00 | 55.84 |
| 1417 | CG | ARG | A | 283 | 43.542 | 97.708 | 48.605 | 1.00 | 54.52 |
| 1418 | CG | ARG | A | 283 | 43.178 | 96.402 | 47.906 | 1.00 | 51.88 |
| 1419 | CD | ARG | A | 283 | 41.756 | 96.413 | 47.337 | 1.00 | 49.72 |
| 1420 | NE | ARG | A | 283 | 41.498 | 95.221 | 46.541 | 1.00 | 48.92 |
| 1421 | CZ | ARG | A | 283 | 41.090 | 94.048 | 47.018 | 1.00 | 51.55 |
| 1422 | NH1 | ARG | A | 283 | 40.859 | 93.877 | 48.316 | 1.00 | 47.52 |
| 1423 | NH2 | ARG | A | 283 | 40.959 | 93.016 | 46.191 | 1.00 | 52.92 |
| 1424 | C | ARG | A | 283 | 46.028 | 97.630 | 48.380 | 1.00 | 55.27 |
| 1425 | O | ARG | A | 283 | 46.725 | 96.621 | 48.266 | 1.00 | 56.54 |
| 1426 | N | PHE | A | 284 | 46.229 | 98.722 | 47.653 | 1.00 | 53.35 |
| 1427 | CA | PHE | A | 284 | 47.300 | 98.807 | 46.663 | 1.00 | 52.84 |
| 1428 | CB | PHE | A | 284 | 47.365 | 100.234 | 46.131 | 1.00 | 50.60 |
| 1429 | CG | PHE | A | 284 | 48.427 | 100.461 | 45.099 | 1.00 | 50.49 |
| 1430 | CD1 | PHE | A | 284 | 48.534 | 99.633 | 43.988 | 1.00 | 51.41 |
| 1431 | CD2 | PHE | A | 284 | 49.271 | 101.565 | 45.194 | 1.00 | 50.80 |
| 1432 | CE1 | PHE | A | 284 | 49.465 | 99.904 | 42.977 | 1.00 | 52.26 |
| 1433 | CE2 | PHE | A | 284 | 50.201 | 101.845 | 44.198 | 1.00 | 51.08 |
| 1434 | CZ | PHE | A | 284 | 50.300 | 101.015 | 43.083 | 1.00 | 51.50 |
| 1435 | C | PHE | A | 284 | 48.649 | 98.407 | 47.253 | 1.00 | 54.00 |
| 1436 | O | PHE | A | 284 | 49.451 | 97.724 | 46.606 | 1.00 | 53.98 |
| 1437 | N | GLY | A | 285 | 48.886 | 98.842 | 48.488 | 1.00 | 54.59 |
| 1438 | CA | GLY | A | 285 | 50.127 | 98.544 | 49.173 | 1.00 | 52.37 |
| 1439 | C | GLY | A | 285 | 50.201 | 97.111 | 49.637 | 1.00 | 53.62 |
| 1440 | O | GLY | A | 285 | 51.255 | 96.492 | 49.508 | 1.00 | 53.97 |
| 1441 | N | GLU | A | 286 | 49.107 | 96.579 | 50.187 | 1.00 | 54.24 |
| 1442 | CA | GLU | A | 286 | 49.105 | 95.195 | 50.640 | 1.00 | 55.89 |
| 1443 | CB | GLU | A | 286 | 47.748 | 94.801 | 51.243 | 1.00 | 59.00 |
| 1444 | CG | GLU | A | 286 | 47.543 | 95.261 | 52.689 | 1.00 | 73.48 |
| 1445 | CD | GLU | A | 286 | 48.488 | 94.578 | 53.691 | 1.00 | 82.58 |
| 1446 | OE1 | GLU | A | 286 | 49.660 | 94.314 | 53.333 | 1.00 | 87.77 |
| 1447 | OE2 | GLU | A | 286 | 48.057 | 94.316 | 54.847 | 1.00 | 87.95 |
| 1448 | C | GLU | A | 286 | 49.444 | 94.305 | 49.449 | 1.00 | 54.80 |
| 1449 | O | GLU | A | 286 | 50.154 | 93.317 | 49.585 | 1.00 | 55.24 |
| 1450 | N | LEU | A | 287 | 48.957 | 94.674 | 48.272 | 1.00 | 54.63 |
| 1451 | CA | LEU | A | 287 | 49.220 | 93.904 | 47.062 | 1.00 | 53.07 |
| 1452 | CG | LEU | A | 287 | 48.328 | 94.385 | 45.911 | 1.00 | 53.27 |
| 1453 | CG | LEU | A | 287 | 46.847 | 94.013 | 45.994 | 1.00 | 53.62 |
| 1454 | CD1 | LEU | A | 287 | 46.093 | 94.548 | 44.798 | 1.00 | 53.04 |
| 1455 | CD2 | LEU | A | 287 | 46.733 | 92.522 | 46.044 | 1.00 | 52.80 |
| 1456 | C | LEU | A | 287 | 50.681 | 93.963 | 46.626 | 1.00 | 53.32 |
| 1457 | O | LEU | A | 287 | 51.277 | 92.934 | 46.326 | 1.00 | 54.68 |
| 1458 | N | LEU | A | 288 | 51.265 | 95.160 | 46.576 | 1.00 | 52.06 |
| 1459 | CA | LEU | A | 288 | 52.661 | 95.281 | 46.168 | 1.00 | 49.18 |
| 1460 | CG | LEU | A | 288 | 53.051 | 96.744 | 45.984 | 1.00 | 45.85 |
| 1461 | CG | LEU | A | 288 | 52.358 | 97.509 | 44.851 | 1.00 | 46.91 |
| 1462 | CD1 | LEU | A | 288 | 52.994 | 98.880 | 44.753 | 1.00 | 43.72 |
| 1463 | CD2 | LEU | A | 288 | 52.488 | 96.772 | 43.516 | 1.00 | 45.08 |
| 1464 | C | LEU | A | 288 | 53.617 | 94.615 | 47.151 | 1.00 | 48.19 |
| 1465 | O | LEU | A | 288 | 54.652 | 94.087 | 46.746 | 1.00 | 48.43 |
| 1466 | N | LEU | A | 289 | 53.278 | 94.627 | 48.435 | 1.00 | 46.56 |
| 1467 | CA | LEU | A | 289 | 54.141 | 94.004 | 49.430 | 1.00 | 47.53 |
| 1468 | CB | LEU | A | 289 | 53.735 | 94.425 | 50.842 | 1.00 | 47.91 |
| 1469 | CG | LEU | A | 289 | 54.173 | 95.850 | 51.191 | 1.00 | 48.14 |
| 1470 | CD1 | LEU | A | 289 | 53.703 | 96.203 | 52.590 | 1.00 | 45.88 |
| 1471 | CD2 | LEU | A | 289 | 55.686 | 95.954 | 51.071 | 1.00 | 42.68 |
| 1472 | C | LEU | A | 289 | 54.129 | 92.495 | 49.321 | 1.00 | 48.39 |
| 1473 | O | LEU | A | 289 | 54.729 | 91.794 | 50.136 | 1.00 | 49.06 |
| 1474 | N | LEU | A | 290 | 53.443 | 92.005 | 48.301 | 1.00 | 49.93 |
| 1475 | CA | LEU | A | 290 | 53.335 | 90.573 | 48.049 | 1.00 | 50.20 |
| 1476 | CB | LEU | A | 290 | 52.000 | 90.286 | 47.362 | 1.00 | 50.79 |
| 1477 | CG | LEU | A | 290 | 50.955 | 89.327 | 47.947 | 1.00 | 53.52 |
| 1478 | CD1 | LEU | A | 290 | 50.772 | 89.553 | 49.436 | 1.00 | 49.98 |
| 1479 | CD2 | LEU | A | 290 | 49.634 | 89.525 | 47.182 | 1.00 | 49.11 |
| 1480 | C | LEU | A | 290 | 54.492 | 90.142 | 47.144 | 1.00 | 48.95 |
| 1481 | O | LEU | A | 290 | 54.855 | 88.975 | 47.098 | 1.00 | 49.77 |
| 1482 | N | LEU | A | 291 | 55.084 | 91.109 | 46.449 | 1.00 | 48.49 |
| 1483 | CA | LEU | A | 291 | 56.172 | 90.847 | 45.517 | 1.00 | 48.02 |
| 1484 | CB | LEU | A | 291 | 56.376 | 92.076 | 44.611 | 1.00 | 45.04 |
| 1485 | CG | LEU | A | 291 | 55.123 | 92.577 | 43.861 | 1.00 | 42.75 |
| 1486 | CD1 | LEU | A | 291 | 55.475 | 93.803 | 43.053 | 1.00 | 42.83 |
| 1487 | CD2 | LEU | A | 291 | 54.572 | 91.512 | 42.937 | 1.00 | 41.80 |
| 1488 | C | LEU | A | 291 | 57.504 | 90.402 | 46.124 | 1.00 | 49.56 |
| 1489 | O | LEU | A | 291 | 58.227 | 89.611 | 45.522 | 1.00 | 51.70 |
| 1490 | N | PRO | A | 292 | 57.874 | 90.916 | 47.308 | 1.00 | 50.50 |
| 1491 | CD | PRO | A | 292 | 57.456 | 92.140 | 48.012 | 1.00 | 51.22 |
| 1492 | CA | PRO | A | 292 | 59.160 | 90.434 | 47.826 | 1.00 | 50.24 |
| 1493 | CB | PRO | A | 292 | 59.450 | 91.388 | 48.988 | 1.00 | 48.90 |
| 1494 | CG | PRO | A | 292 | 58.786 | 92.659 | 48.549 | 1.00 | 49.56 |
| 1495 | C | PRO | A | 292 | 59.023 | 88.984 | 48.282 | 1.00 | 50.05 |
| 1496 | O | PRO | A | 292 | 59.977 | 88.208 | 48.253 | 1.00 | 52.57 |
| 1497 | N | THR | A | 293 | 57.820 | 88.636 | 48.710 | 1.00 | 48.75 |
| 1498 | CA | THR | A | 293 | 57.508 | 87.289 | 49.159 | 1.00 | 49.98 |
| 1499 | CB | THR | A | 293 | 56.091 | 87.240 | 49.777 | 1.00 | 49.37 |
| 1500 | OG1 | THR | A | 293 | 56.059 | 88.045 | 50.959 | 1.00 | 49.17 |
| 1501 | CG2 | THR | A | 293 | 55.716 | 85.829 | 50.136 | 1.00 | 50.42 |
| 1502 | C | THR | A | 293 | 57.557 | 86.342 | 47.958 | 1.00 | 50.49 |
| 1503 | O | THR | A | 293 | 58.160 | 85.268 | 48.012 | 1.00 | 50.26 |
| 1504 | N | LEU | A | 294 | 56.904 | 86.754 | 46.880 | 1.00 | 50.54 |
| 1505 | CA | LEU | A | 294 | 56.858 | 85.980 | 45.653 | 1.00 | 50.07 |
| 1506 | CB | LEU | A | 294 | 56.065 | 86.760 | 44.588 | 1.00 | 50.37 |
| 1507 | CG | LEU | A | 294 | 55.887 | 86.255 | 43.147 | 1.00 | 50.02 |
| 1508 | CD1 | LEU | A | 294 | 55.294 | 84.862 | 43.141 | 1.00 | 49.21 |
| 1509 | CD2 | LEU | A | 294 | 54.967 | 87.204 | 42.393 | 1.00 | 50.16 |
| 1510 | C | LEU | A | 294 | 58.281 | 85.716 | 45.177 | 1.00 | 50.36 |
| 1511 | O | LEU | A | 294 | 58.593 | 84.627 | 44.711 | 1.00 | 51.04 |
| 1512 | N | GLN | A | 295 | 59.144 | 86.719 | 45.315 | 1.00 | 51.63 |
| 1513 | CA | GLN | A | 295 | 60.540 | 86.623 | 44.882 | 1.00 | 53.37 |
| 1514 | CB | GLN | A | 295 | 61.218 | 87.983 | 45.041 | 1.00 | 54.61 |
| 1515 | CG | GLN | A | 295 | 62.405 | 88.225 | 44.137 | 1.00 | 59.44 |
| 1516 | CD | GLN | A | 295 | 63.079 | 89.575 | 44.404 | 1.00 | 63.10 |
| 1517 | OE1 | GLN | A | 295 | 63.698 | 90.162 | 43.514 | 1.00 | 66.10 |
| 1518 | NE2 | GLN | A | 295 | 62.971 | 90.061 | 45.637 | 1.00 | 63.79 |
| 1519 | C | GLN | A | 295 | 61.266 | 85.585 | 45.723 | 1.00 | 53.60 |
| 1520 | O | GLN | A | 295 | 61.934 | 84.699 | 45.197 | 1.00 | 53.68 |
| 1521 | N | SER | A | 296 | 61.116 | 85.706 | 47.036 | 1.00 | 53.49 |
| 1522 | CA | SER | A | 296 | 61.740 | 84.795 | 47.981 | 1.00 | 52.16 |
| 1523 | CB | SER | A | 296 | 61.284 | 85.146 | 49.398 | 1.00 | 52.03 |
| 1524 | OG | SER | A | 296 | 61.652 | 84.137 | 50.317 | 1.00 | 53.93 |
| 1525 | C | SER | A | 296 | 61.411 | 83.333 | 47.666 | 1.00 | 51.43 |
| 1526 | O | SER | A | 296 | 62.308 | 82.511 | 47.490 | 1.00 | 49.71 |
| 1527 | N | ILE | A | 297 | 60.128 | 83.003 | 47.588 | 1.00 | 50.05 |
| 1528 | CA | ILE | A | 297 | 59.748 | 81.628 | 47.305 | 1.00 | 49.62 |
| 1529 | CB | ILE | A | 297 | 58.226 | 81.434 | 47.398 | 1.00 | 45.90 |
| 1530 | CG2 | ILE | A | 297 | 57.895 | 79.967 | 47.361 | 1.00 | 44.96 |
| 1531 | CG1 | ILE | A | 297 | 57.710 | 81.997 | 48.720 | 1.00 | 44.15 |
| 1532 | CD1 | ILE | A | 297 | 56.204 | 82.079 | 48.800 | 1.00 | 42.04 |
| 1533 | C | ILE | A | 297 | 60.233 | 81.165 | 45.931 | 1.00 | 51.40 |
| 1534 | O | ILE | A | 297 | 60.556 | 79.996 | 45.755 | 1.00 | 52.67 |
| 1535 | N | THR | A | 298 | 60.302 | 82.076 | 44.967 | 1.00 | 53.15 |
| 1536 | CA | THR | A | 298 | 60.749 | 81.721 | 43.615 | 1.00 | 54.69 |
| 1537 | CB | THR | A | 298 | 60.502 | 82.879 | 42.601 | 1.00 | 52.86 |
| 1538 | OG1 | THR | A | 298 | 59.101 | 83.164 | 42.524 | 1.00 | 50.37 |
| 1539 | CG2 | THR | A | 298 | 61.007 | 82.499 | 41.223 | 1.00 | 48.43 |
| 1540 | C | THR | A | 298 | 62.234 | 81.355 | 43.589 | 1.00 | 56.55 |
| 1541 | O | THR | A | 298 | 62.640 | 80.417 | 42.901 | 1.00 | 57.43 |
| 1542 | N | TRP | A | 299 | 63.047 | 82.101 | 44.327 | 1.00 | 59.09 |
| 1543 | CA | TRP | A | 299 | 64.475 | 81.818 | 44.376 | 1.00 | 62.16 |
| 1544 | CB | TRP | A | 299 | 65.216 | 82.913 | 45.151 | 1.00 | 67.64 |
| 1545 | CG | TRP | A | 299 | 65.532 | 84.128 | 44.315 | 1.00 | 76.18 |
| 1546 | CD2 | TRP | A | 299 | 66.778 | 84.841 | 44.261 | 1.00 | 81.84 |
| 1547 | CE2 | TRP | A | 299 | 66.622 | 85.888 | 43.320 | 1.00 | 82.99 |
| 1548 | CE3 | TRP | A | 299 | 68.027 | 84.672 | 44.891 | 1.00 | 84.69 |
| 1549 | CD1 | TRP | A | 299 | 64.689 | 84.771 | 43.450 | 1.00 | 77.11 |
| 1550 | NE1 | TRP | A | 299 | 65.335 | 85.828 | 42.851 | 1.00 | 80.02 |
| 1551 | CZ2 | TRP | A | 299 | 67.659 | 86.798 | 43.017 | 1.00 | 85.95 |
| 1552 | CZ3 | TRP | A | 299 | 69.064 | 85.579 | 44.589 | 1.00 | 86.00 |
| 1553 | CH2 | TRP | A | 299 | 68.874 | 86.618 | 43.648 | 1.00 | 85.57 |
| 1554 | C | TRP | A | 299 | 64.700 | 80.462 | 45.031 | 1.00 | 61.65 |
| 1555 | O | TRP | A | 299 | 65.591 | 79.714 | 44.629 | 1.00 | 63.42 |
| 1556 | N | GLN | A | 300 | 63.884 | 80.138 | 46.028 | 1.00 | 59.32 |
| 1557 | CA | GLN | A | 300 | 64.001 | 78.861 | 46.718 | 1.00 | 58.51 |
| 1558 | CG | GLN | A | 300 | 63.068 | 78.828 | 47.926 | 1.00 | 56.83 |
| 1559 | CG | GLN | A | 300 | 63.482 | 77.805 | 48.962 | 1.00 | 57.88 |
| 1560 | CD | GLN | A | 300 | 62.511 | 77.692 | 50.122 | 1.00 | 59.40 |
| 1561 | OE1 | GLN | A | 300 | 62.034 | 78.697 | 50.646 | 1.00 | 58.26 |
| 1562 | NE2 | GLN | A | 300 | 62.224 | 76.459 | 50.539 | 1.00 | 59.18 |
| 1563 | C | GLN | A | 300 | 63.665 | 77.701 | 45.766 | 1.00 | 59.30 |
| 1564 | O | GLN | A | 300 | 64.366 | 76.691 | 45.727 | 1.00 | 60.14 |
| 1565 | N | MET | A | 301 | 62.591 | 77.863 | 44.999 | 1.00 | 58.87 |
| 1566 | CA | MET | A | 301 | 62.158 | 76.860 | 44.039 | 1.00 | 57.31 |
| 1567 | CB | MET | A | 301 | 60.825 | 77.287 | 43.413 | 1.00 | 58.61 |
| 1568 | CG | MET | A | 301 | 60.389 | 76.450 | 42.204 | 1.00 | 59.91 |
| 1569 | SD | MET | A | 301 | 58.776 | 76.899 | 41.540 | 1.00 | 59.27 |
| 1570 | CE | MET | A | 301 | 59.226 | 78.115 | 40.338 | 1.00 | 56.06 |
| 1571 | C | MET | A | 301 | 63.197 | 76.641 | 42.942 | 1.00 | 57.88 |
| 1572 | O | MET | A | 301 | 63.482 | 75.505 | 42.572 | 1.00 | 57.16 |
| 1573 | N | ILE | A | 302 | 63.754 | 77.726 | 42.408 | 1.00 | 58.60 |
| 1574 | CA | ILE | A | 302 | 64.751 | 77.605 | 41.349 | 1.00 | 60.24 |
| 1575 | CG | ILE | A | 302 | 65.049 | 78.969 | 40.690 | 1.00 | 59.46 |
| 1576 | CG2 | ILE | A | 302 | 66.269 | 78.864 | 39.791 | 1.00 | 58.10 |
| 1577 | CG1 | ILE | A | 302 | 63.840 | 79.414 | 39.868 | 1.00 | 60.85 |
| 1578 | CD1 | ILE | A | 302 | 64.092 | 80.665 | 39.058 | 1.00 | 63.07 |
| 1579 | C | ILE | A | 302 | 66.050 | 76.995 | 41.853 | 1.00 | 61.33 |
| 1580 | O | ILE | A | 302 | 66.632 | 76.145 | 41.191 | 1.00 | 62.22 |
| 1581 | N | GLU | A | 303 | 66.509 | 77.440 | 43.016 | 1.00 | 63.68 |
| 1582 | CA | GLU | A | 303 | 67.727 | 76.903 | 43.595 | 1.00 | 66.07 |
| 1583 | CB | GLU | A | 303 | 67.994 | 77.538 | 44.956 | 1.00 | 70.83 |
| 1584 | CG | GLU | A | 303 | 68.698 | 78.887 | 44.896 | 1.00 | 77.80 |
| 1585 | CD | GLU | A | 303 | 68.865 | 79.503 | 46.276 | 1.00 | 82.92 |
| 1586 | OE1 | GLU | A | 303 | 68.894 | 78.730 | 47.260 | 1.00 | 86.16 |
| 1587 | OE2 | GLU | A | 303 | 68.975 | 80.749 | 46.379 | 1.00 | 86.20 |
| 1588 | C | GLU | A | 303 | 67.516 | 75.414 | 43.762 | 1.00 | 66.02 |
| 1589 | O | GLU | A | 303 | 68.409 | 74.613 | 43.493 | 1.00 | 66.48 |
| 1590 | N | GLN | A | 304 | 66.318 | 75.046 | 44.199 | 1.00 | 65.78 |
| 1591 | CA | GLN | A | 304 | 65.975 | 73.645 | 44.390 | 1.00 | 66.11 |
| 1592 | CB | GLN | A | 304 | 64.619 | 73.529 | 45.076 | 1.00 | 64.66 |
| 1593 | CG | GLN | A | 304 | 64.242 | 72.117 | 45.419 | 1.00 | 66.16 |
| 1594 | CD | GLN | A | 304 | 63.078 | 72.066 | 46.362 | 1.00 | 67.71 |
| 1595 | OE1 | GLN | A | 304 | 62.889 | 72.975 | 47.169 | 1.00 | 69.18 |
| 1596 | NE2 | GLN | A | 304 | 62.295 | 70.996 | 46.286 | 1.00 | 68.50 |
| 1597 | C | GLN | A | 304 | 65.963 | 72.884 | 43.058 | 1.00 | 66.79 |
| 1598 | O | GLN | A | 304 | 66.448 | 71.766 | 42.973 | 1.00 | 68.04 |
| 1599 | N | ILE | A | 305 | 65.402 | 73.481 | 42.017 | 1.00 | 67.53 |
| 1600 | CA | ILE | A | 305 | 65.389 | 72.832 | 40.716 | 1.00 | 68.72 |
| 1601 | CB | ILE | A | 305 | 64.563 | 73.652 | 39.692 | 1.00 | 68.43 |
| 1602 | CG2 | ILE | A | 305 | 64.756 | 73.104 | 38.290 | 1.00 | 67.26 |
| 1603 | CG1 | ILE | A | 305 | 63.084 | 73.613 | 40.070 | 1.00 | 67.67 |
| 1604 | CD1 | ILE | A | 305 | 62.201 | 74.462 | 39.183 | 1.00 | 66.18 |
| 1605 | C | ILE | A | 305 | 66.838 | 72.736 | 40.227 | 1.00 | 70.29 |
| 1606 | O | ILE | A | 305 | 67.240 | 71.762 | 39.602 | 1.00 | 68.88 |
| 1607 | N | GLN | A | 306 | 67.628 | 73.754 | 40.533 | 1.00 | 73.16 |
| 1608 | CA | GLN | A | 306 | 69.010 | 73.768 | 40.099 | 1.00 | 77.84 |
| 1609 | CB | GLN | A | 306 | 69.626 | 75.150 | 40.346 | 1.00 | 79.91 |
| 1610 | CG | GLN | A | 306 | 70.999 | 75.385 | 39.704 | 1.00 | 85.69 |
| 1611 | CD | GLN | A | 306 | 71.185 | 74.731 | 38.320 | 1.00 | 90.51 |
| 1612 | OE1 | GLN | A | 306 | 71.815 | 75.310 | 37.431 | 1.00 | 91.97 |
| 1613 | NE2 | GLN | A | 306 | 70.668 | 73.514 | 38.147 | 1.00 | 92.14 |
| 1614 | C | GLN | A | 306 | 69.844 | 72.681 | 40.761 | 1.00 | 79.80 |
| 1615 | O | GLN | A | 306 | 70.781 | 72.150 | 40.168 | 1.00 | 80.34 |
| 1616 | N | PHE | A | 307 | 69.506 | 72.333 | 41.991 | 1.00 | 81.72 |
| 1617 | CA | PHE | A | 307 | 70.260 | 71.299 | 42.659 | 1.00 | 83.32 |
| 1618 | CB | PHE | A | 307 | 70.065 | 71.414 | 44.175 | 1.00 | 87.37 |
| 1619 | CG | PHE | A | 307 | 69.411 | 70.232 | 44.765 | 1.00 | 92.13 |
| 1620 | CD1 | PHE | A | 307 | 70.160 | 69.108 | 45.078 | 1.00 | 94.03 |
| 1621 | CD2 | PHE | A | 307 | 68.024 | 70.161 | 44.823 | 1.00 | 94.32 |
| 1622 | CE1 | PHE | A | 307 | 69.542 | 67.932 | 45.415 | 1.00 | 95.95 |
| 1623 | CE2 | PHE | A | 307 | 67.390 | 68.988 | 45.157 | 1.00 | 95.95 |
| 1624 | CZ | PHE | A | 307 | 68.145 | 67.863 | 45.452 | 1.00 | 95.95 |
| 1625 | C | PHE | A | 307 | 69.773 | 69.940 | 42.124 | 1.00 | 83.33 |
| 1626 | O | PHE | A | 307 | 70.576 | 69.128 | 41.669 | 1.00 | 83.75 |
| 1627 | N | VAL | A | 308 | 68.461 | 69.711 | 42.153 | 1.00 | 83.05 |
| 1628 | CA | VAL | A | 308 | 67.874 | 68.454 | 41.686 | 1.00 | 83.61 |
| 1629 | CB | VAL | A | 308 | 66.317 | 68.526 | 41.663 | 1.00 | 83.31 |
| 1630 | CG1 | VAL | A | 308 | 65.766 | 67.751 | 40.492 | 1.00 | 85.10 |
| 1631 | CG2 | VAL | A | 308 | 65.747 | 67.935 | 42.934 | 1.00 | 81.91 |
| 1632 | C | VAL | A | 308 | 68.373 | 68.077 | 40.300 | 1.00 | 84.94 |
| 1633 | O | VAL | A | 308 | 68.405 | 66.900 | 39.942 | 1.00 | 85.00 |
| 1634 | N | LYS | A | 309 | 68.764 | 69.074 | 39.517 | 1.00 | 86.39 |
| 1635 | CA | LYS | A | 309 | 69.260 | 68.804 | 38.178 | 1.00 | 88.28 |
| 1636 | CB | LYS | A | 309 | 69.170 | 70.042 | 37.293 | 1.00 | 87.33 |
| 1637 | CG | LYS | A | 309 | 69.751 | 69.807 | 35.918 | 1.00 | 85.44 |
| 1638 | CD | LYS | A | 309 | 69.948 | 71.095 | 35.174 | 1.00 | 85.63 |
| 1639 | CE | LYS | A | 309 | 70.436 | 70.826 | 33.765 | 1.00 | 87.76 |
| 1640 | NZ | LYS | A | 309 | 70.538 | 72.085 | 32.963 | 1.00 | 90.34 |
| 1641 | C | LYS | A | 309 | 70.705 | 68.327 | 38.182 | 1.00 | 90.63 |
| 1642 | O | LYS | A | 309 | 71.014 | 67.288 | 37.593 | 1.00 | 92.50 |
| 1643 | N | LEU | A | 3iW | 71.587 | 69.085 | 38.833 | 1.00 | 91.71 |
| 1644 | CA | LEU | A | 310 | 72.998 | 68.725 | 38.877 | 1.00 | 92.34 |
| 1645 | CB | LEU | A | 310 | 73.850 | 69.920 | 39.299 | 1.00 | 93.54 |
| 1646 | CG | LEU | A | 310 | 74.249 | 70.728 | 38.056 | 1.00 | 95.95 |
| 1647 | CD1 | LEU | A | 310 | 75.178 | 71.870 | 38.440 | 1.00 | 95.95 |
| 1648 | CD2 | LEU | A | 310 | 74.941 | 69.794 | 37.042 | 1.00 | 95.95 |
| 1649 | C | LEU | A | 310 | 73.338 | 67.509 | 39.720 | 1.00 | 92.16 |
| 1650 | O | LEU | A | 310 | 74.417 | 66.941 | 39.576 | 1.00 | 91.31 |
| 1651 | N | PHE | A | 311 | 72.426 | 67.109 | 40.597 | 1.00 | 92.42 |
| 1652 | CA | PHE | A | 311 | 72.649 | 65.913 | 41.398 | 1.00 | 92.90 |
| 1653 | CB | PHE | A | 311 | 72.229 | 66.141 | 42.851 | 1.00 | 91.58 |
| 1654 | CG | PHE | A | 311 | 73.263 | 66.877 | 43.652 | 1.00 | 92.43 |
| 1655 | CD1 | PHE | A | 311 | 73.737 | 68.114 | 43.220 | 1.00 | 93.18 |
| 1656 | CD2 | PHE | A | 311 | 73.808 | 66.319 | 44.801 | 1.00 | 92.88 |
| 1657 | CE1 | PHE | A | 311 | 74.743 | 68.783 | 43.920 | 1.00 | 93.14 |
| 1658 | CE2 | PHE | A | 311 | 74.818 | 66.982 | 45.510 | 1.00 | 93.31 |
| 1659 | CZ | PHE | A | 311 | 75.286 | 68.215 | 45.066 | 1.00 | 92.91 |
| 1660 | C | PHE | A | 311 | 71.869 | 64.774 | 40.750 | 1.00 | 93.93 |
| 1661 | O | PHE | A | 311 | 71.419 | 63.837 | 41.414 | 1.00 | 94.85 |
| 1662 | N | GLY | A | 312 | 71.727 | 64.897 | 39.428 | 1.00 | 94.22 |
| 1663 | CA | GLY | A | 312 | 71.044 | 63.919 | 38.600 | 1.00 | 93.73 |
| 1664 | C | GLY | A | 312 | 69.678 | 63.410 | 39.011 | 1.00 | 94.03 |
| 1665 | O | GLY | A | 312 | 69.062 | 62.664 | 38.253 | 1.00 | 94.80 |
| 1666 | N | MET | A | 313 | 69.200 | 63.800 | 40.191 | 1.00 | 94.19 |
| 1667 | CA | MET | A | 313 | 67.898 | 63.346 | 40.679 | 1.00 | 94.78 |
| 1668 | CB | MET | A | 313 | 67.451 | 64.214 | 41.856 | 1.00 | 94.23 |
| 1669 | CG | MET | A | 313 | 67.888 | 63.660 | 43.194 | 1.00 | 95.95 |
| 1670 | SD | MET | A | 313 | 67.866 | 64.835 | 44.561 | 1.00 | 95.95 |
| 1671 | CE | MET | A | 313 | 69.656 | 64.990 | 44.848 | 1.00 | 95.71 |
| 1672 | C | MET | A | 313 | 66.792 | 63.304 | 39.626 | 1.00 | 95.63 |
| 1673 | O | MET | A | 313 | 66.192 | 62.251 | 39.389 | 1.00 | 95.95 |
| 1674 | N | VAL | A | 314 | 66.524 | 64.444 | 38.993 | 1.00 | 94.89 |
| 1675 | CA | VAL | A | 314 | 65.482 | 64.517 | 37.976 | 1.00 | 93.46 |
| 1676 | CB | VAL | A | 314 | 64.325 | 65.435 | 38.441 | 1.00 | 93.61 |
| 1677 | CG1 | VAL | A | 314 | 63.173 | 65.379 | 37.441 | 1.00 | 93.50 |
| 1678 | CG2 | VAL | A | 314 | 63.863 | 65.026 | 39.838 | 1.00 | 91.98 |
| 1679 | C | VAL | A | 314 | 66.012 | 65.044 | 36.643 | 1.00 | 92.87 |
| 1680 | O | VAL | A | 314 | 67.050 | 65.708 | 36.590 | 1.00 | 91.94 |
| 1681 | N | ALA | A | 315 | 65.298 | 64.725 | 35.566 | 1.00 | 92.16 |
| 1682 | CA | ALA | A | 315 | 65.664 | 65.185 | 34.231 | 1.00 | 91.57 |
| 1683 | CB | ALA | A | 315 | 65.191 | 64.187 | 33.174 | 1.00 | 90.93 |
| 1684 | C | ALA | A | 315 | 64.936 | 66.511 | 34.068 | 1.00 | 91.09 |
| 1685 | O | ALA | A | 315 | 63.792 | 66.643 | 34.508 | 1.00 | 91.28 |
| 1686 | N | ILE | A | 316 | 65.576 | 67.496 | 33.449 | 1.00 | 89.78 |
| 1687 | CA | ILE | A | 316 | 64.913 | 68.777 | 33.301 | 1.00 | 88.77 |
| 1688 | CB | ILE | A | 316 | 65.621 | 69.837 | 34.156 | 1.00 | 88.28 |
| 1689 | CG2 | ILE | A | 316 | 65.300 | 71.241 | 33.673 | 1.00 | 88.35 |
| 1690 | CG1 | ILE | A | 316 | 65.157 | 69.656 | 35.601 | 1.00 | 86.07 |
| 1691 | CD1 | ILE | A | 316 | 65.941 | 70.428 | 36.591 | 1.00 | 87.92 |
| 1692 | C | ILE | A | 316 | 64.634 | 69.261 | 31.884 | 1.00 | 89.20 |
| 1693 | O | ILE | A | 316 | 65.495 | 69.749 | 31.150 | 1.00 | 88.66 |
| 1694 | N | ASP | A | 317 | 63.357 | 69.078 | 31.568 | 1.00 | 89.57 |
| 1695 | CA | ASP | A | 317 | 62.634 | 69.372 | 30.335 | 1.00 | 90.01 |
| 1696 | CB | ASP | A | 317 | 61.193 | 69.645 | 30.754 | 1.00 | 92.59 |
| 1697 | CG | ASP | A | 317 | 60.810 | 68.877 | 32.032 | 1.00 | 95.95 |
| 1698 | CD1 | ASP | A | 317 | 60.123 | 67.834 | 31.927 | 1.00 | 95.95 |
| 1699 | OD2 | ASP | A | 317 | 61.219 | 69.301 | 33.144 | 1.00 | 95.95 |
| 1700 | C | ASP | A | 317 | 63.099 | 70.421 | 29.320 | 1.00 | 89.07 |
| 1701 | O | ASP | A | 317 | 62.320 | 70.811 | 28.446 | 1.00 | 88.70 |
| 1702 | N | ASN | A | 318 | 64.347 | 70.864 | 29.435 | 1.00 | 87.57 |
| 1703 | CA | ASN | A | 318 | 64.963 | 71.831 | 28.520 | 1.00 | 87.04 |
| 1704 | CB | ASN | A | 318 | 65.213 | 71.178 | 27.155 | 1.00 | 88.75 |
| 1705 | CG | ASN | A | 318 | 66.631 | 71.413 | 26.650 | 1.00 | 90.71 |
| 1706 | OD1 | ASN | A | 318 | 67.060 | 72.557 | 26.474 | 1.00 | 91.48 |
| 1707 | ND2 | ASN | A | 318 | 67.369 | 70.328 | 26.423 | 1.00 | 89.69 |
| 1708 | C | ASN | A | 318 | 64.292 | 73.185 | 28.309 | 1.00 | 86.45 |
| 1709 | O | ASN | A | 318 | 64.977 | 74.169 | 28.009 | 1.00 | 86.63 |
| 1710 | N | LEU | A | 319 | 62.970 | 73.259 | 28.415 | 1.00 | 85.40 |
| 1711 | CA | LEU | A | 319 | 62.333 | 74.562 | 28.272 | 1.00 | 84.07 |
| 1712 | CB | LEU | A | 319 | 60.829 | 74.444 | 27.984 | 1.00 | 83.22 |
| 1713 | CG | LEU | A | 319 | 60.122 | 75.778 | 27.676 | 1.00 | 81.79 |
| 1714 | CD1 | LEU | A | 319 | 60.798 | 76.470 | 26.497 | 1.00 | 80.23 |
| 1715 | CD2 | LEU | A | 319 | 58.650 | 75.534 | 27.378 | 1.00 | 80.40 |
| 1716 | C | LEU | A | 319 | 62.566 | 75.201 | 29.637 | 1.00 | 83.18 |
| 1717 | O | LEU | A | 319 | 62.877 | 76.391 | 29.743 | 1.00 | 82.68 |
| 1718 | N | LEU | A | 320 | 62.449 | 74.379 | 30.681 | 1.00 | 81.52 |
| 1719 | CA | LEU | A | 320 | 62.655 | 74.846 | 32.042 | 1.00 | 81.00 |
| 1720 | CB | LEU | A | 320 | 62.433 | 73.713 | 33.050 | 1.00 | 77.38 |
| 1721 | CG | LEU | A | 320 | 61.195 | 72.812 | 32.990 | 1.00 | 74.93 |
| 1722 | CD1 | LEU | A | 320 | 60.811 | 72.470 | 34.414 | 1.00 | 71.44 |
| 1723 | CD2 | LEU | A | 320 | 60.029 | 73.483 | 32.286 | 1.00 | 74.69 |
| 1724 | C | LEU | A | 320 | 64.070 | 75.396 | 32.200 | 1.00 | 82.36 |
| 1725 | O | LEU | A | 320 | 64.288 | 76.350 | 32.940 | 1.00 | 83.83 |
| 1726 | N | GLN | A | 321 | 65.032 | 74.795 | 31.511 | 1.00 | 83.98 |
| 1727 | CA | GLN | A | 321 | 66.411 | 75.259 | 31.591 | 1.00 | 85.38 |
| 1728 | CB | GLN | A | 321 | 67.355 | 74.283 | 30.905 | 1.00 | 89.31 |
| 1729 | CG | GLN | A | 321 | 67.435 | 72.913 | 31.535 | 1.00 | 93.36 |
| 1730 | CD | GLN | A | 321 | 68.209 | 71.941 | 30.660 | 1.00 | 95.80 |
| 1731 | OE1 | GLN | A | 321 | 68.845 | 71.016 | 31.158 | 1.00 | 95.95 |
| 1732 | NE2 | GLN | A | 321 | 68.147 | 72.145 | 29.343 | 1.00 | 95.95 |
| 1733 | C | GLN | A | 321 | 66.537 | 76.602 | 30.903 | 1.00 | 85.17 |
| 1734 | O | GLN | A | 321 | 67.101 | 77.537 | 31.457 | 1.00 | 84.45 |
| 1735 | N | GLU | A | 322 | 66.018 | 76.692 | 29.683 | 1.00 | 85.66 |
| 1736 | CA | GLU | A | 322 | 66.088 | 77.939 | 28.930 | 1.00 | 86.89 |
| 1737 | CB | GLU | A | 322 | 65.425 | 77.788 | 27.558 | 1.00 | 89.68 |
| 1738 | CG | GLU | A | 322 | 66.068 | 76.760 | 26.635 | 1.00 | 94.53 |
| 1739 | CD | GLU | A | 322 | 65.478 | 76.793 | 25.224 | 1.00 | 95.95 |
| 1740 | OE1 | GLU | A | 322 | 64.294 | 77.187 | 25.088 | 1.00 | 95.95 |
| 1741 | OE2 | GLU | A | 322 | 66.189 | 76.414 | 24.258 | 1.00 | 95.95 |
| 1742 | C | GLU | A | 322 | 65.423 | 79.100 | 29.666 | 1.00 | 85.42 |
| 1743 | O | GLU | A | 322 | 65.972 | 80.204 | 29.715 | 1.00 | 84.52 |
| 1744 | N | MET | A | 323 | 64.250 | 78.845 | 30.245 | 1.00 | 83.85 |
| 1745 | CA | MET | A | 323 | 63.505 | 79.889 | 30.941 | 1.00 | 82.06 |
| 1746 | CB | MET | A | 323 | 62.025 | 79.778 | 30.586 | 1.00 | 83.70 |
| 1747 | CG | MET | A | 323 | 61.752 | 80.018 | 29.119 | 1.00 | 86.33 |
| 1748 | SD | MET | A | 323 | 60.007 | 80.113 | 28.772 | 1.00 | 92.36 |
| 1749 | CE | MET | A | 323 | 60.024 | 80.880 | 27.173 | 1.00 | 93.45 |
| 1750 | C | MET | A | 323 | 63.650 | 80.017 | 32.457 | 1.00 | 79.66 |
| 1751 | O | MET | A | 323 | 63.590 | 81.124 | 32.980 | 1.00 | 79.03 |
| 1752 | N | LEU | A | 324 | 63.834 | 78.912 | 33.168 | 1.00 | 77.18 |
| 1753 | CA | LEU | A | 324 | 63.969 | 78.993 | 34.620 | 1.00 | 75.17 |
| 1754 | CB | LEU | A | 324 | 63.250 | 77.816 | 35.295 | 1.00 | 70.52 |
| 1755 | CG | LEU | A | 324 | 61.731 | 77.702 | 35.180 | 1.00 | 65.13 |
| 1756 | CD1 | LEU | A | 324 | 61.252 | 76.517 | 35.992 | 1.00 | 61.92 |
| 1757 | CD2 | LEU | A | 324 | 61.085 | 78.971 | 35.674 | 1.00 | 64.50 |
| 1758 | C | LEU | A | 324 | 65.413 | 79.042 | 35.123 | 1.00 | 76.78 |
| 1759 | O | LEU | A | 324 | 65.693 | 79.663 | 36.152 | 1.00 | 75.06 |
| 1760 | N | LEU | A | 325 | 66.329 | 78.401 | 34.399 | 1.00 | 79.20 |
| 1761 | CA | LEU | A | 325 | 67.733 | 78.344 | 34.815 | 1.00 | 81.55 |
| 1762 | CB | LEU | A | 325 | 68.185 | 76.881 | 34.833 | 1.00 | 78.53 |
| 1763 | CG | LEU | A | 325 | 67.215 | 75.944 | 35.564 | 1.00 | 76.77 |
| 1764 | CD1 | LEU | A | 325 | 67.669 | 74.506 | 35.413 | 1.00 | 76.38 |
| 1765 | CD2 | LEU | A | 325 | 67.127 | 76.332 | 37.031 | 1.00 | 75.85 |
| 1766 | C | LEU | A | 325 | 68.710 | 79.191 | 33.983 | 1.00 | 85.38 |
| 1767 | O | LEU | A | 325 | 69.290 | 80.151 | 34.489 | 1.00 | 85.61 |
| 1768 | N | GLY | A | 326 | 68.903 | 78.832 | 32.717 | 1.00 | 89.93 |
| 1769 | CA | GLY | A | 326 | 69.809 | 79.591 | 31.862 | 1.00 | 92.77 |
| 1770 | C | GLY | A | 326 | 70.395 | 78.763 | 30.729 | 1.00 | 94.69 |
| 1771 | O | GLY | A | 326 | 71.369 | 78.037 | 30.927 | 1.00 | 95.95 |
| 1772 | N | GLY | A | 327 | 69.812 | 78.872 | 29.538 | 1.00 | 95.44 |
| 1773 | CA | GLY | A | 327 | 70.304 | 78.104 | 28.404 | 1.00 | 95.95 |
| 1774 | C | GLY | A | 327 | 69.866 | 78.638 | 27.048 | 1.00 | 95.95 |
| 1775 | O | GLY | A | 327 | 69.493 | 79.830 | 26.957 | 1.00 | 95.95 |
| 1776 | OXT | GLY | A | 327 | 69.907 | 77.866 | 26.063 | 1.00 | 95.95 |
| 1777 | | GLY | A | 327 | | | | | |
| 1778 | O | HOH | W | 1 | 45.353 | 102.993 | 32.467 | 1.00 | 50.15 |
| 1779 | O | HOH | W | 2 | 47.316 | 82.519 | 34.627 | 1.00 | 57.09 |
| 1780 | O | HOH | W | 3 | 31.392 | 64.065 | 45.689 | 1.00 | 60.13 |
| 1781 | O | HOH | W | 4 | 46.892 | 91.480 | 19.212 | 1.00 | 66.69 |
| 1782 | O | HOH | W | 5 | 64.831 | 89.601 | 40.555 | 1.00 | 46.68 |
| 1783 | O | HOH | W | 6 | 45.378 | 77.628 | 33.925 | 1.00 | 63.84 |
| 1784 | O | HOH | W | 7 | 48.658 | 89.782 | 54.328 | 1.00 | 74.42 |
| 1785 | O | HOH | W | 8 | 38.225 | 90.198 | 35.001 | 1.00 | 94.21 |
| 1786 | O | HOH | W | 9 | 67.358 | 103.472 | 22.826 | 1.00 | 65.96 |
| 1787 | O | HOH | W | 10 | 40.781 | 105.542 | 59.632 | 1.00 | 57.47 |
| 1788 | O | HOH | W | 11 | 64.373 | 76.920 | 22.211 | 1.00 | 90.22 |
| 1789 | O | HOH | W | 12 | 65.998 | 86.720 | 46.709 | 1.00 | 59.52 |
| 1790 | O | HOH | W | 13 | 37.481 | 88.829 | 39.254 | 1.00 | 52.14 |
| 1791 | O | HOH | W | 14 | 63.610 | 91.916 | 41.126 | 1.00 | 57.56 |
| 1792 | O | HOH | W | 15 | 38.719 | 91.362 | 23.684 | 1.00 | 64.77 |
| 1793 | | HOH | W | 15 | | | | | |
| 1794 | C1 | PLM | A | 328 | 51.604 | 75.192 | 37.410 | 1.00 | 85.43 |
| 1795 | O1 | PLM | A | 328 | 50.976 | 74.110 | 37.329 | 1.00 | 84.59 |
| 1796 | O2 | PLM | A | 328 | 51.199 | 76.231 | 36.857 | 1.00 | 86.9S |
| 1797 | C2 | PLM | A | 328 | 52.897 | 75.263 | 38.236 | 1.00 | 84.45 |
| 1798 | C3 | PLM | A | 328 | 52.585 | 75.314 | 39.72S | 1.00 | 81.28 |
| 1799 | C4 | PLM | A | 328 | 53.818 | 75.421 | 40.617 | 1.00 | 82.41 |
| 1800 | C5 | PLM | A | 328 | 53.431 | 75.459 | 42.122 | 1.00 | 80.70 |
| 1801 | C6 | PLM | A | 328 | 53.881 | 74.176 | 42.753 | 1.00 | 80.20 |
| 1802 | C7 | PLM | A | 328 | 55.285 | 74.378 | 43.205 | 1.00 | 81.28 |
| 1803 | C8 | PLM | A | 328 | 55.933 | 73.139 | 43.737 | 1.00 | 82.12 |
| 1804 | C9 | PLM | A | 328 | 57.331 | 73.563 | 44.086 | 1.00 | 83.S6 |
| 1805 | CA | PLM | A | 328 | 58.225 | 72.448 | 44.493 | 1.00 | 84.66 |
| 1806 | CB | PLM | A | 328 | 59.214 | 72.437 | 43.346 | 1.00 | 86.04 |
| 1807 | CC | PLM | A | 328 | 60.264 | 71.394 | 43.405 | 1.00 | 86.77 |
| 1808 | CD | PLM | A | 328 | 61.123 | 71.558 | 42.177 | 1.00 | 90.12 |
| 1809 | CE | PLM | A | 328 | 62.197 | 70.505 | 42.125 | 1.00 | 92.94 |
| 1810 | CF | PLM | A | 328 | 61.694 | 69.314 | 41.329 | 1.00 | 95.95 |
| 1811 | CG | PLM | A | 328 | 62.733 | 68.218 | 41.256 | 1.00 | 95.95 |

TABLE 4
Data Summary Of Analytes Detected By
GC/MS Using Chemical Ionization

| | | Predicted Mass Of | |
|---|---|---|---|
| Peak | [M + H]+ | Free Acid (Da) | Identification/Comments |
| a | 243 | 228 | myristic acid |
| b | 269 | 254 | likely mono-unsaturated palmitic |
| | | | acid |
| c | 271 | 256 | palmitic acid |
| d | 283 | 268 | idenfication pending |
| e | 297 | 282 | likely mono-unsaturated stearic |
| | | | acid |
| f | 299 | 284 | stearic acid |
| g | 311 | 296 | identification pending |

It will be understood that various details of the invention can be changed without departing from the scope of the invention. Furthermore, the foregoing description is for the purpose of illustration only, and not for the purpose of limitation—the invention being defined by the claims.

1. A substantially pure HNF4γ ligand binding domain polypeptide in crystalline form.

2. The polypeptide of claim 1, wherein the crystalline form has lattice constants of a=152.71 Å, b=152.71 Å, c=93.42 Å, α=90°, β=90°, γ=90°.

3. The polypeptide of claim 1, wherein the crystalline form is a tetragonal crystalline form.

4. The polypeptide of claim 1, wherein the crystalline form has a space group of 14,22.

5. The polypeptide of claim 1, wherein the HNF4γ ligand binding domain polypeptide has the amino acid sequence shown in SEQ ID NO:4.

6. The polypeptide of claim 1, wherein the HNF4γ ligand binding domain polypeptide is in complex with a ligand.

7. The polypeptide of claim 6, wherein the ligand is a fatty acid.

8. The polypeptide of claim 7, wherein the fatty acid is selected from the group consisting of lauristic acid, myristic acid, palmitic acid, stearic acid, mono-unsaturated analogs of palmitic acid, mono-unsaturated analogs of stearic acid.

9. The polypeptide of claim 1, wherein the HNF4γ ligand binding domain has a crystalline structure further characterized by the coordinates corresponding to Table 2.

10. The polypeptide of claim 1, wherein the crystalline form contains one HNF4γ ligand binding domain polypeptide in the asymmetric unit.

11. The polypeptide of claim 1, wherein the crystalline form is such that the three-dimensional structure of the crystallized HNF4γ ligand binding domain polypeptide can be determined to a resolution of about 3 Å or better.

12. The polypeptide of claim 10, wherein the crystalline form contains one or more atoms having an atomic weight of 40 grams/mol or greater.

13. A method for determining the three-dimensional structure of a crystallized HNF4γ ligand binding domain polypeptide to a resolution of about 3 Å or better, the method comprising:
(a) crystallizing an HNF4γ ligand binding domain polypeptide; and
(b) analyzing the HNF4γ ligand binding domain polypeptide to determine the three-dimensional structure of the crystallized HNF4γ ligand binding domain polypeptide, whereby the three-dimensional structure of a crystallized HNF4γ ligand binding domain polypeptide is determined to a resolution of about 3 Å or better.

14. The method of claim 13, wherein the analyzing is by X-ray diffraction.

15. The method of claim 13, wherein the crystallization is accomplished by the hanging drop vapor diffusion method, and wherein the HNF4γ ligand binding domain is mixed with an equal volume of reservoir.

16. The method of claim 15, wherein the reservoir comprises 0.75 M ammonium phosphate pH=5.0-5.5 and 10 mM DTT.

17. The method of claim 15, wherein the reservoir comprises 0.7-1.0 M sodium or potassium phosphate pH 5.0-6.0.

18. A method of generating a crystallized HNF4γ ligand binding domain polypeptide, the method comprising:
(a) incubating a solution comprising an HNF4γ ligand binding domain with an equal volume of reservoir; and
(b) crystallizing the HNF4γ ligand binding domain polypeptide using the hanging drop method, whereby a crystallized HNF4γ ligand binding domain polypeptide is generated.

19. A crystallized HNF4γ ligand binding domain polypeptide produced by the method of claim 18.

20. A method of designing a modulator of an HNF4 polypeptide, the method comprising:
(a) designing a potential modulator of an HNF4 polypeptide that will form bonds with amino acids in a substrate binding site based upon a crystalline structure of an HNF4γ ligand binding domain polypeptide;
(b) synthesizing the modulator; and
(c) determining whether the potential modulator modulates the activity of the HNF4 polypeptide, whereby a modulator of an HNF4 polypeptide is designed.

21. A method of designing a modulator that selectively modulates the activity of an HNF4 polypeptide, the method comprising:
(a) obtaining a crystalline form of an HNF4γ ligand binding domain polypeptide;
(b) evaluating the three-dimensional structure of the crystallized HNF4γ ligand binding domain polypeptide; and
(c) synthesizing a potential modulator based on the three-dimensional crystal structure of the crystallized HNF4γ ligand binding domain polypeptide, whereby a modulator that selectively modulates the activity of an HNF4 polypeptide is designed.

22. The method of claim 21, wherein the method further comprises contacting an HNF4γ ligand binding domain polypeptide with the potential modulator; and assaying the HNF4γ ligand binding domain polypeptide for binding of the potential modulator, for a change in activity of the HNF4γ ligand binding domain polypeptide, or both.

23. The method of claim 21, wherein the crystalline form is in tetragonal form.

24. The method of claim 23, wherein the crystalline form is such that the three-dimensional structure of the crystallized HNF4γ ligand binding domain polypeptide can be determined to a resolution of about 3 Å or better.

25. A method for identifying an HNF4 modulator, the method comprising:
(a) providing atomic coordinates of an HNF4γ ligand binding domain to a computerized modeling system; and
(b) modeling ligands that fit spatially into the binding pocket of the HNF4γ ligand binding domain, whereby an HNF4 modulator is identified.

26. The method of claim 25, wherein the method further comprises identifying in an assay for HNF4-mediated activity a modeled ligand that increases or decreases the activity of the HNF4.

27. A method of identifying an HNF4γ modulator that selectively modulates the activity of an HNF4γ polypeptide compared to other polypeptides, the method comprising:
(a) providing atomic coordinates of an HNF4γ ligand binding domain to a computerized modeling system; and
(b) modeling a ligand that fits into the binding pocket of an HNF4γ ligand binding domain and that interacts with conformationally constrained residues of an HNF4γ that are conserved among HNF4 isoforms, whereby an HNF4γ modulator is identified.

28. The method of claim 27, wherein the method further comprises identifying in a biological assay for HNF4γ mediated activity a modeled ligand that selectively binds to the HNF4γ ligand binding domain and increases or decreases the activity of the HNF4γ.

29. A method of designing a modulator of an HNF4 polypeptide, the method comprising:
(a) selecting a candidate HNF4 ligand;
(b) determining which amino acid or amino acids of an HNF4 polypeptide interact with the ligand using a three-dimensional model of a crystallized protein comprising an HNF4γ LBD;
(c) identifying in a biological assay for HNF4 activity a degree to which the ligand modulates the activity of the HNF4 polypeptide;
(d) selecting a chemical modification of the ligand wherein the interaction between the amino acids of the HNF4 polypeptide and the ligand is predicted to be modulated by the chemical modification;
(e) performing the chemical modification on the ligand to form a modified ligand;
(f) contacting the modified ligand with the HNF4 polypeptide;
(g) identifying in a biological assay for HNF4 activity a degree to which the modified ligand modulates the biological activity of the HNF4 polypeptide; and
(h) comparing the biological activity of the HNF4 polypeptide in the presence of modified ligand with the biological activity of the HNF4 polypeptide in the presence of the unmodified ligand, whereby a modulator of an HNF4 polypeptide is designed.

30. The method of claim 29, wherein the HNF4 polypeptide is an HNF4γ polypeptide.

31. The method of claim 29, wherein the three-dimensional model of a crystallized protein is an HNF4γ LBD polypeptide with a bound ligand.

32. The method of claim 31, wherein the ligand is a fatty acid.

33. The method of claim 32, wherein the fatty acid is palmitic acid.

34. The method of claim 29, wherein the method further comprises repeating steps (a) through (f), if the biological activity of the HNF4 polypeptide in the presence of the modified ligand varies from the biological activity of the HNF4 polypeptide in the presence of the unmodified ligand.

35. An assay method for identifying a compound that inhibits binding of a ligand to an HNF4 polypeptide, the assay method comprising:
(a) incubating an HNF4 polypeptide with a ligand in the presence of a test inhibitor compound;
(b) determining an amount of ligand that is bound to the HNF4 polypeptide, wherein decreased binding of ligand to the HNF4 protein in the presence of the test inhibitor compound relative to binding of ligand in the absence of the test inhibitor compound is indicative of inhibition; and
(c) identifying the test compound as an inhibitor of ligand binding if decreased ligand binding is observed, whereby a compound that inhibits binding of a ligand to an HNF4 polypeptide is identified.

36. The method of claim 35, wherein the ligand is a fatty acid.

37. The method of claim 36, wherein the fatty acid is selected from the group consisting of lauristic acid, myristic acid, palmitic acid, stearic acid, mono-unsaturated analogs of palmitic acid, mono-unsaturated analogs of stearic acid.
